(19) United States
(12) Patent Application Publication
(10) Pub. No.: US 2020/0071351 A1
(43) Pub. Date: Mar. 5, 2020

(54) TACCALONOLIDE MICROTUBULE STABILIZERS

(71) Applicants: Antonius Ola, Naikoten I Kupang (ID); Samantha S.M. Yee, San Antonio, TX (US); THE BOARD OF REGENTS OF THE UNIVERSITY OF TEXAS SYSTEM, Austin, TX (US); THE BOARD OF REGENTS OF THE UNIVERSITY OF OKLAHOMA, Norman, OK (US)

(72) Inventors: Susan L. Mooberry, San Antonio, TX (US); April L. Risinger, San Antonio, TX (US); Robert H. Cichewicz, Norman, OK (US); Lin Du, Norman, OK (US); Jing Li, Columbia, MD (US); Jiangnan Peng, Columbia, MD (US); Antonius Ola, Naikoten I Kupang (ID); Samantha S.M. Yee, San Antonio, TX (US)

(21) Appl. No.: 16/469,064

(22) PCT Filed: Dec. 15, 2017
(86) PCT No.: PCT/US2017/066779

§ 371 Date: Jun. 12, 2019

Related U.S. Application Data
(60) Provisional application No. 62/434,919, filed on Dec. 15, 2016.

(51) Int. Cl. C07J 71/00; C07J 17/00; A61P 35/00

(52) U.S. Cl. CPC C07J 71/0005; A61P 35/00; C07J 17/00

(57) ABSTRACT

This present disclosure relates to the fields of medicine and pharmaceuticals. In particular, the invention relates to the identification of epoxytaccalonolide microtubule stabilizers for use in inhibiting cell proliferation and disrupting normal cellular microtubule processes leading to cell death. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/434,919, filed on Dec. 15, 2016, which is incorporated herein by reference in its entirety.

### ACKNOWLEDGEMENT

This invention was made with government support under grant no. CA121138, awarded by the National Institutes of Health. The government has certain rights in the invention.

### BACKGROUND

Microtubules are cellular structures important for normal cellular metabolism, cellular transport and cell division. Interrupting microtubule-dependent processes causes cellular defects including inhibition of proliferation and cellular trafficking leading to initiation of cell death pathways. Microtubule disrupting agents including microtubule stabilizers are one of the most important classes of anticancer therapeutics used in the clinic today. Additionally microtubule stabilizers are used in other human diseases of hyperproliferation including cardiovascular disease, where they are used to coat stents. The taxoid microtubule stabilizer paclitaxel (Taxol™) has been widely used in the treatment of solid tumors, including breast, ovarian and lung cancers for over a decade as a single agent and in combination with targeted therapies. In spite of their clinical utility, the shortcomings of paclitaxel and the second generation semi-synthetic taxoid, docetaxel (Taxotere™), include innate and acquired drug resistance and dose limiting toxicities (Fojo and Menefee, 2007). Two new microtubule stabilizers have been approved for clinical use in the past few years: the epothilone ixabepilone (Ixempra) and the taxoid cabazitaxel (Jevtana), which circumvent some, but not all of the shortcomings of first and second generation microtubule stabilizers (Morris and Formier, 2008; Galsky et al., 2010, Shen et al., 2011). These microtubule stabilizing drugs all bind to the interior lumen of the intact microtubule at the taxoid binding site, which causes a stabilization of microtubule protofilament interactions and thereby decreases the dynamic nature of microtubules (Nogales et al., 1995).

Two additional classes of microtubule stabilizers have been isolated from nature: laulimalides/peloruside A and the taccalonolides. Laulimalide and peloruside A have been shown to bind to the exterior of the microtubule at a site distinct from the taxoid binding site, yet result in microtubule stabilization effects nearly identical to the taxoids (Bennett et al., 2010). The microtubule stabilizing properties of the taccalonolides A, E, B and N together with their ability to overcome multiple clinically relevant mechanisms of drug resistance (Risinger et al., 2008) prompted further interest in identifying new taccalonolides.

Intense efforts over the past three decades have identified a large variety of interesting chemical compounds from the roots and rhizomes of Tacca species, including 25 taccalonolides, denoted as taccalonolides A-Y (Chen et al., 1987; Chen et al., 1988; Shen et al., 1991; Shen et al., 1996; Chen et al., 1997; WO/2001/040256; Huang and Liu, 2002; Muhlbauer et al., 2003; Yang et al., 2008). However, there were limited biological studies on the taccalonolides. In 2003, microtubule stabilizing activities of taccalonolides A and E were first reported (Tinley et al., 2003). Follow up studies showed preliminary structure-activity relationships (SAR) for the antiproliferative activities of taccalonolides A, E, B and N. The antiproliferative potencies of these four taccalonolides in HeLa cells were all in the mid nanomolar range (190 nM to 644 nM) (Risinger et al., 2008) and further studies showed that the taccalonolides A, E and N have in vivo antitumor activity (Peng et al., 2011). However, a full understanding of the structure-activity relationships of the taccalonolides remains to be elucidated. Given that the biological activity profiles of known taccalonolides are different, and in view of the wide variety of diseases that may be treated or prevented with compounds having potent microtubule stabilization effects, and the high degree of unmet medical need represented within this variety of diseases, it is desirable to synthesize new compounds with diverse structures that may have improved biological activity profiles for the treatment of one or more indications.

### SUMMARY

Thus, in accordance with the present invention, there are provided novel taccalonolide derivatives with microtubule stabilizing properties, pharmaceutical compositions thereof, methods of their manufacture, and methods for their use, including for the prevention and treatment of mammalian cell hyperproliferation and initiation of cell death.

In one aspect, there are provided compounds of the formula:

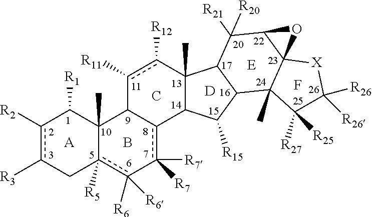

wherein: R₁ is hydroxy, alkoxy_{(C≤12)} or acyloxy_{(C≤12)}; R₂ is hydroxy, halogen, or R₂ is taken together with R₃ to form an epoxide at C-2/C-3; R₃ is hydroxy, halo, or R₂ is taken together with R₃ as defined above; R₅ is hydrogen, hydroxy, amino, alkoxy_{(C≤9)}, alkylamino_{(C≤6)}, or dialkylamino_{(C≤12)}; R₆ is hydrogen, hydroxy, alkoxy_{(C≤30)}, acyloxy_{(C≤30)}, or oxo if R_{6′} is not present; R_{6′} when present is hydrogen or hydroxy, alkoxy_{(C≤30)} or acyloxy_{(C≤30)}; R₇ is hydrogen, hydroxy, alkoxy_{(C≤30)}, acyloxy_{(C≤30)}, or oxo if R_{7′} is not present; R_{7′} when present is hydrogen, hydroxy, alkoxy_{(C≤30)}, or acyloxy_{(C≤30)}; R₁₁ is hydrogen, hydroxy, alkyl_{(C≤6)}, alkoxy_{(C≤8)}, or acyloxy_{(C≤8)}; R₁₂ is hydrogen, hydroxy, alkyl_{(C≤6)}, alkoxy_{(C≤8)}, or acyloxy_{(C≤8)}; R₁₅ is hydrogen, hydroxy, alkyl_{(C≤30)}, alkoxy_{(C≤30)} or acyloxy_{(C≤30)}; R₂₀ is hydrogen, hydroxy, hydroperoxy, alkoxy_{(C≤8)} or acyloxy_{(C≤8)}; R₂₁ is hydrogen or alkyl_{(C≤6)}; R₂₅ is hydrogen, hydroxy, alkoxy_{(C≤8)} or acyloxy_{(C≤8)}; R₂₆ is hydrogen, hydroxy, alkoxy_{(C≤8)} or oxo if R_{26′} is not present; R_{26′} when present is hydrogen, hydroxy or alkoxy_{(C≤8)}; R₂₇ is hydrogen or alkyl_{(C≤6)}; and X is O, NR^{x} or CR^{x}₂, wherein each R^{x} is independently hydrogen or alkyl_{(C≤6)}; or a pharmaceutically acceptable salt thereof.

In one aspect, disclosed are compounds having a structure represented by a formula:

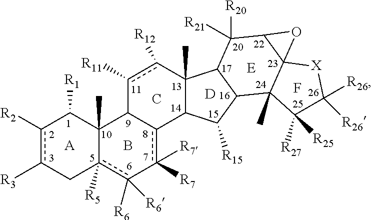

wherein each --- is an optional covalent bond; wherein R₁ is selected from —OH, C1-C12 hydroxy, C1-C12 alkoxy, and —OC(O)(C1-C12 alkyl); wherein each of R₂ and R₃ is independently selected from hydrogen, —OH, C1-C12 hydroxy, and halogen, or wherein R₂ and R₃ together comprise —O—; wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent; wherein each of R₆ and R_{6′} is independently selected from hydrogen, —OH, C1-C30 hydroxy, C1-C30 alkoxy, C1-C30 acyloxy, —OC(O)Ar₁, —OC(O)Ar₂, —OC(O)(C1-C4 alkyl)Ar₂, and —OC(O)(C1-C8 azide); wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; or wherein each of R₆ and R_{6′} together comprise ═O, or wherein one of R₆ and R_{6′} is absent; wherein each of R₇ and R_{7′} is independently selected from hydrogen, —OH, C1-C30 hydroxy, C1-C30 alkoxy, and C1-C30 acyloxy, or wherein each of R₇ and R_{7′} together comprise ═O, or wherein one of R₇ and R_{7′} is absent; wherein each of R₁₁ and R₁₂ is independently selected from hydrogen, —OH, C1-C8 hydroxy, C1-C6 alkyl, C1-C8 alkoxy, and C1-C8 acyloxy; wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, and —OC(O)(C1-C8 azide); wherein each of R₃₁ₐ and R_{31b}, when present, is independently selected from hydrogen and C1-C8 alkyl; wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

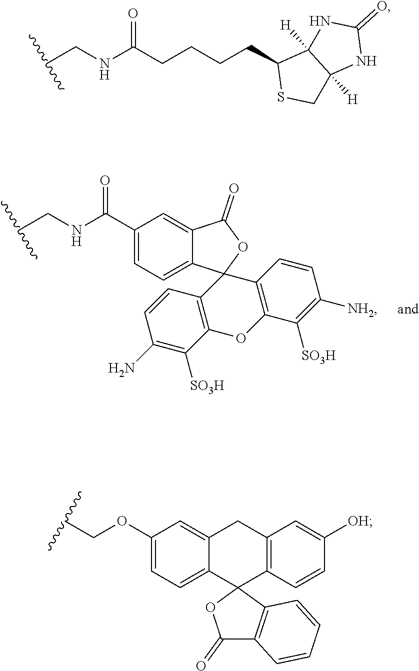

wherein R₂₀ is selected from hydrogen, —OH, —OOH, C1-C8 hydroxy, C1-C8 hydroperoxy, C1-C8 alkoxy, and C1-C8 acyloxy; wherein R₂₁ is selected from hydrogen and C1-C6 alkyl; wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar¹, and —OC(O)(C1-C8 azide); wherein each of R₂₆ and R_{26′} is independently selected from hydrogen, —OH, C1-C8 hydroxy, and C1-C8 alkoxy, or wherein each of R₂₆ and R_{26′} together comprise ═O; wherein R₂₇ is selected from hydrogen and C1-C6 alkyl; and wherein X is selected from O, NR^{x}, and CR^{x}₂; wherein R^{x}, when present, is selected from hydrogen and C1-C6 alkyl, or a pharmaceutically acceptable salt thereof.

In one aspect, disclosed are compounds having a structure represented by a formula:

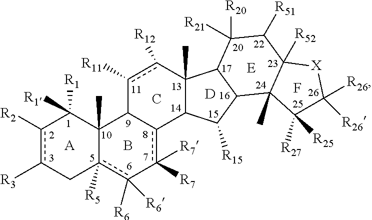

wherein each --- is an optional covalent bond; wherein R₁ is selected from —OH, C1-C12 hydroxy, C1-C12 alkoxy, —OC(O)(C1-C12 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃, and wherein R_{1′} is hydrogen; or wherein each of R₁ and R_{1′} together comprise ═O or ═NR₄₆; wherein each of R₂ and R₃ is independently selected from hydrogen, —OH, C1-C12 hydroxy, and halogen, or wherein R₂ and R₃ together comprise an epoxide at C-2/C-3; wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent; wherein each of R₆ and R_{6′} is independently selected from hydrogen, —OH, C1-C30 hydroxy, C1-C30 alkoxy, C1-C30 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃; or wherein each of R₆ and R_{6′} together comprise ═O or ═NR₄₆, or wherein one of R₆ and R_{6′} is absent; wherein R₇ is selected from hydrogen, —OH, C1-C30 hydroxy, C1-C30 alkoxy, C1-C30 acyloxy, and —OC(O)NR₃₁ₐR_{31b}, and wherein R_{7′} is selected from hydrogen, —OH, C1-C30 hydroxy, C1-C30 alkoxy, and C1-C30 acyloxy; or wherein each of R₇ and R_{7′} together comprise ═O; or wherein one of R₇ and R_{7′} is absent; wherein each of R₁₁ and R₁₂ is independently selected from hydrogen, —OH, C1-C8 hydroxy, C1-C6 alkyl, C1-C8 alkoxy, and C1-C8 acyloxy; wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, —OC(O)(C1-C8 azide), and —OC(O)CH₃; wherein R₂₀ is selected from hydrogen, —OH, —OOH, C1-C8 hydroxy, C1-C8 hydroperoxy, C1-C8 alkoxy, and C1-C8 acyloxy; wherein R₂₁ is selected from hydrogen and C1-C6 alkyl; wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar₁, and —OC(O)(C1-C8 azide); wherein each of R₂₆ and R_{26′} is independently selected from hydrogen, —OH, C1-C8 hydroxy, and C1-C8 alkoxy, or wherein each of R₂₆ and R_{26′} together comprise ═O; wherein R₂₇ is selected from hydrogen and C1-C6 alkyl; and wherein each of R₃₁ₐ and R_{31b}, when present, is independently selected from hydrogen and C1-C12 alkyl; wherein each occurrence of R₄₁, R₄₂, R₄₄, R₄₅ₐ, and R_{45b}, when present, is independently selected from hydrogen and C1-C12 alkyl; wherein each occurrence of R₄₃, when present, is independently selected from hydrogen, C1-C12 alkyl, and monocyclic aryl monosubstituted with a methyl group; wherein each occurrence of R₄₆, when present, is independently selected from hydrogen and C1-C12 alkyl; wherein each of R₅₁ and R₅₂ is independently halogen; or wherein each of R₅₁ and R₅₂ together comprise —O— or —N(R₅₃)—; wherein R₅₃, when present, is selected from hydrogen, C1-C4 alkyl, —SO₂R₅₄, and a structure having a formula:

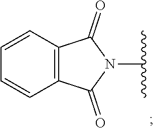

wherein R₅₄, when present, is selected from hydrogen, C1-C4 alkyl, —CH₂CH₂Si(CH₃)₃, and monocyclic aryl monosubstituted with a methyl group; wherein each occurrence of Cy¹, when present, is independently heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

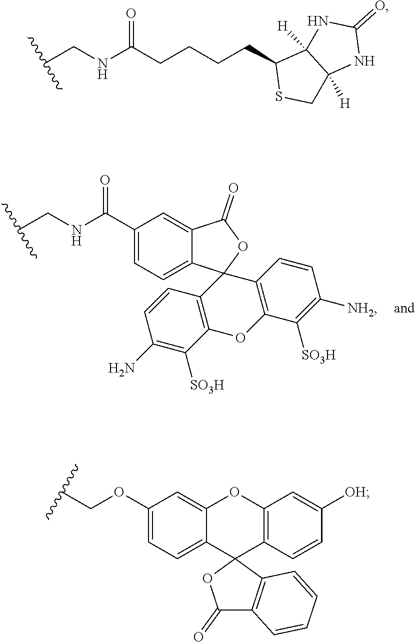

wherein each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein X is selected from O, NR^{x}, and CR^{x}₂; wherein R^{x}, when present, is selected from hydrogen and C1-C6 alkyl, or a pharmaceutically acceptable salt thereof.

In a further aspect, the compounds are further defined as:

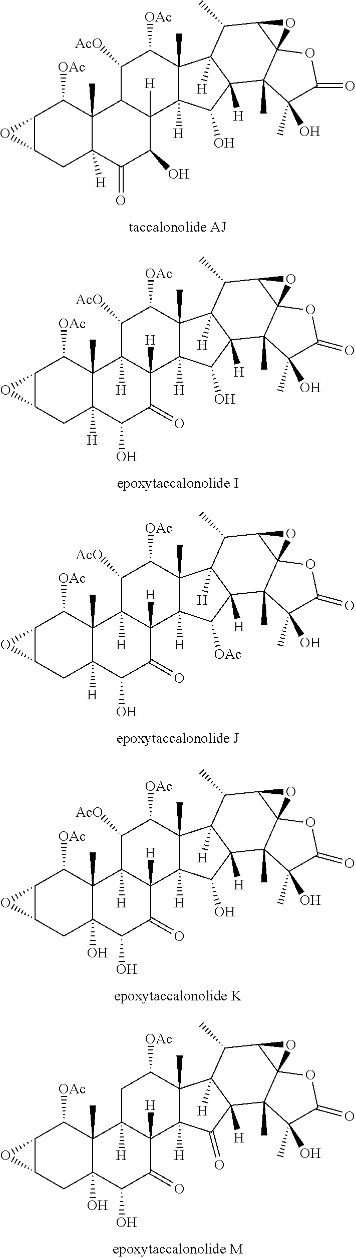

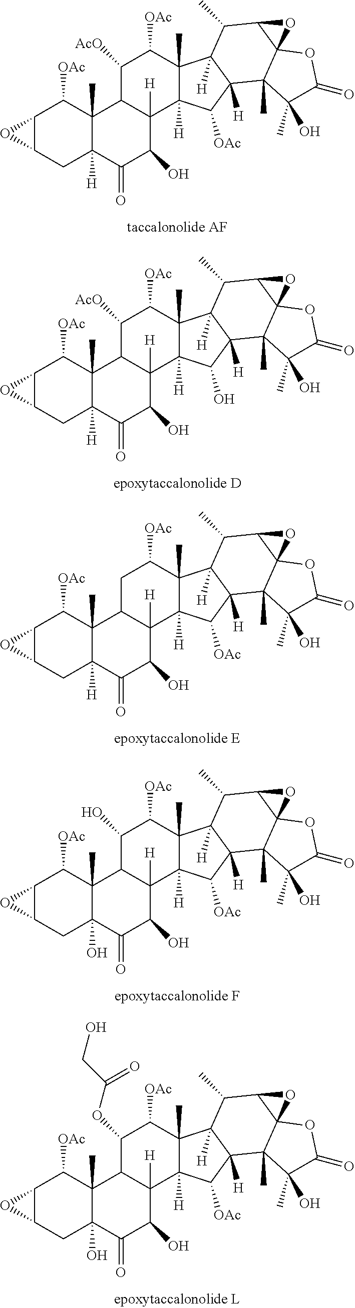

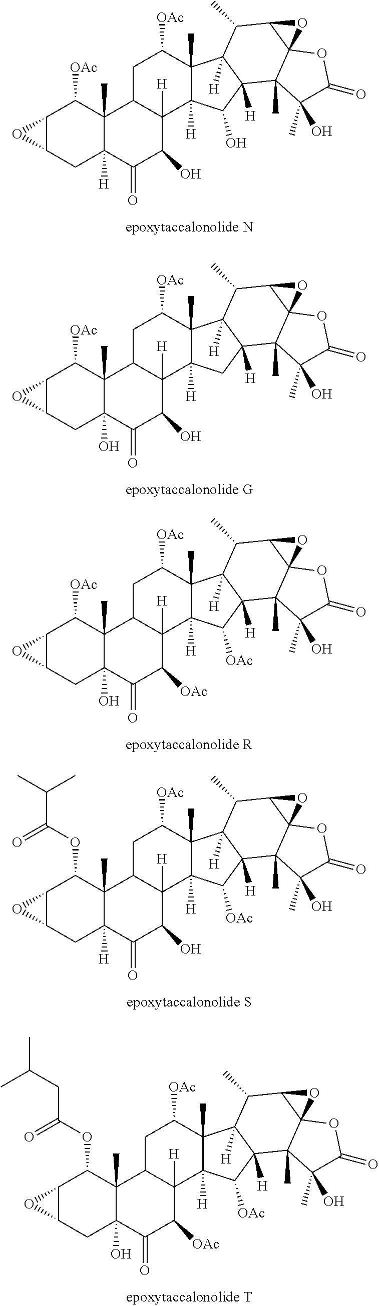

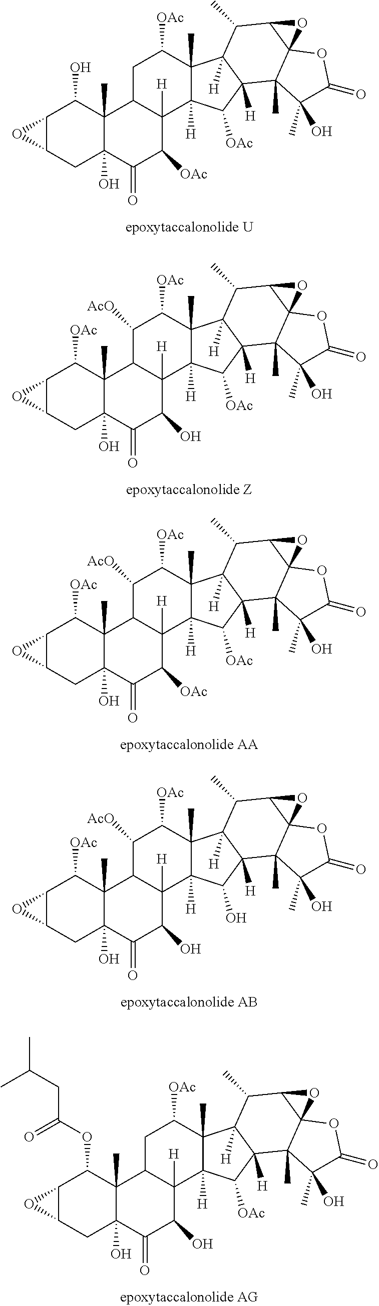

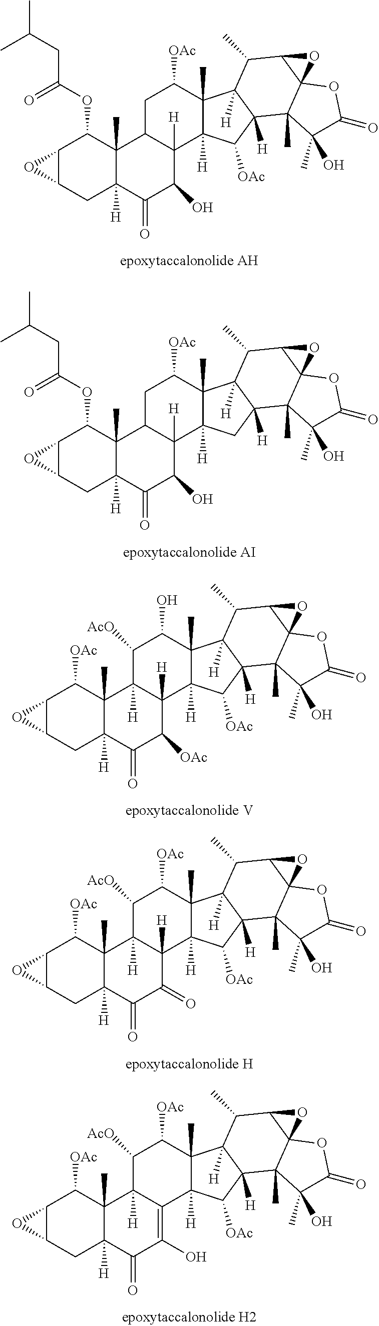

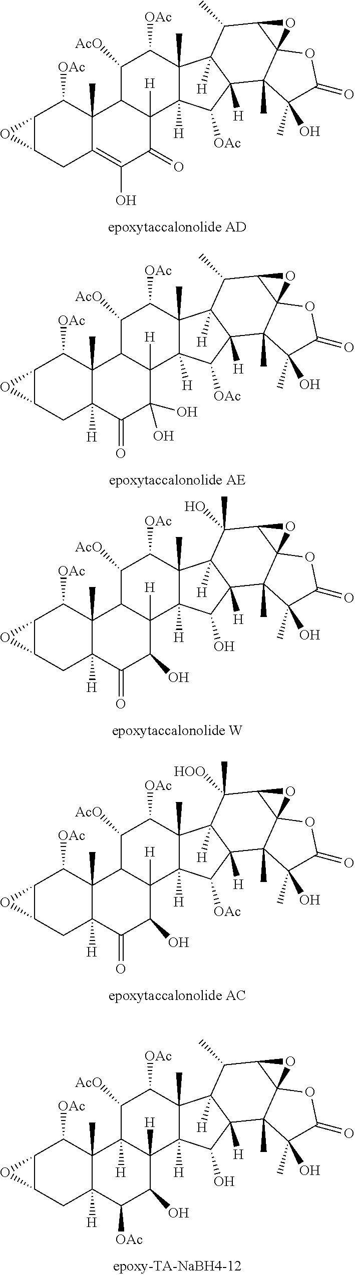

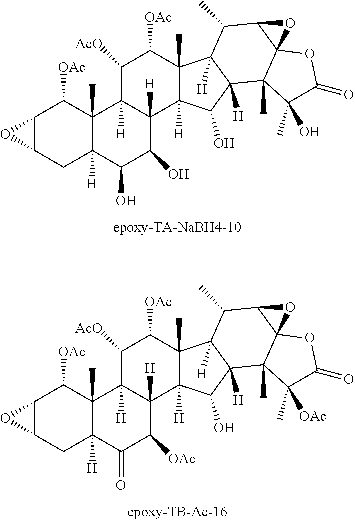

In a further aspect, the compounds are further defined as:

IC₅₀=6 nM

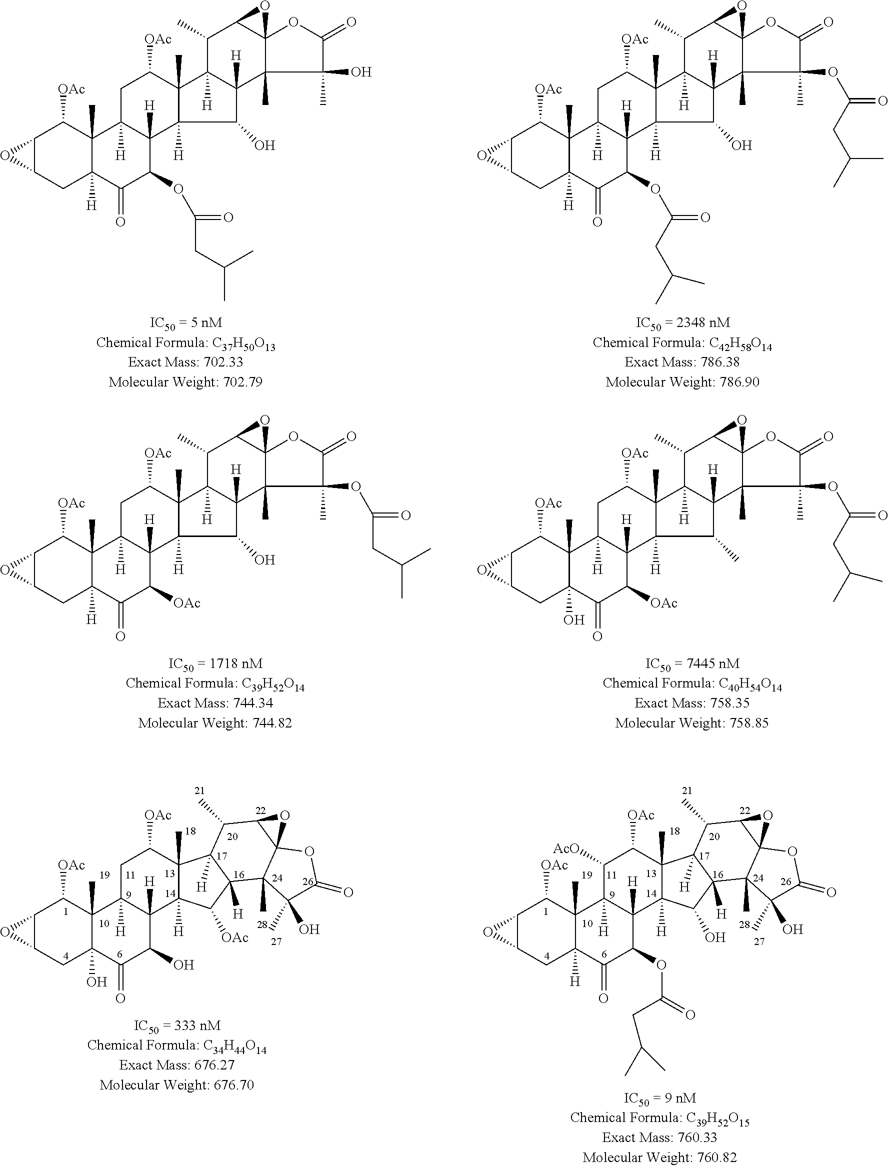

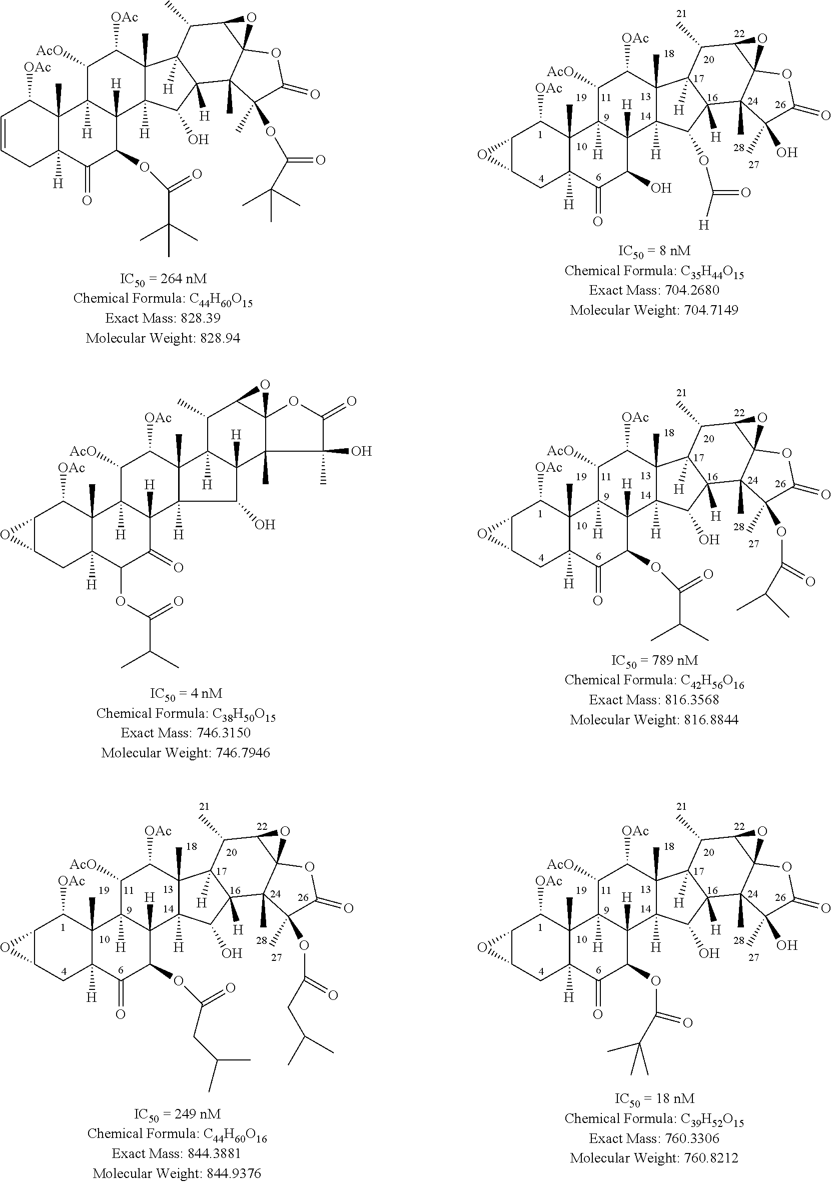

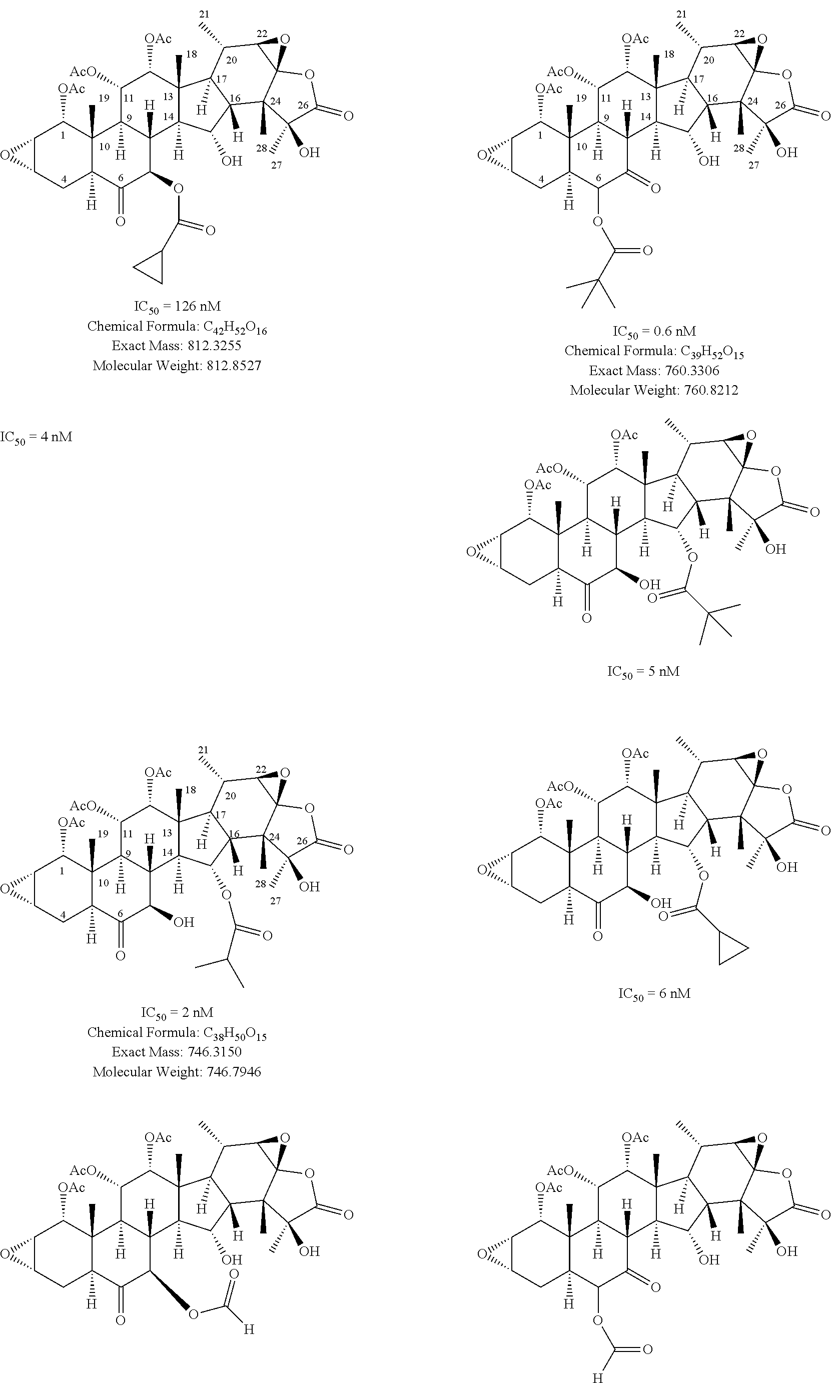

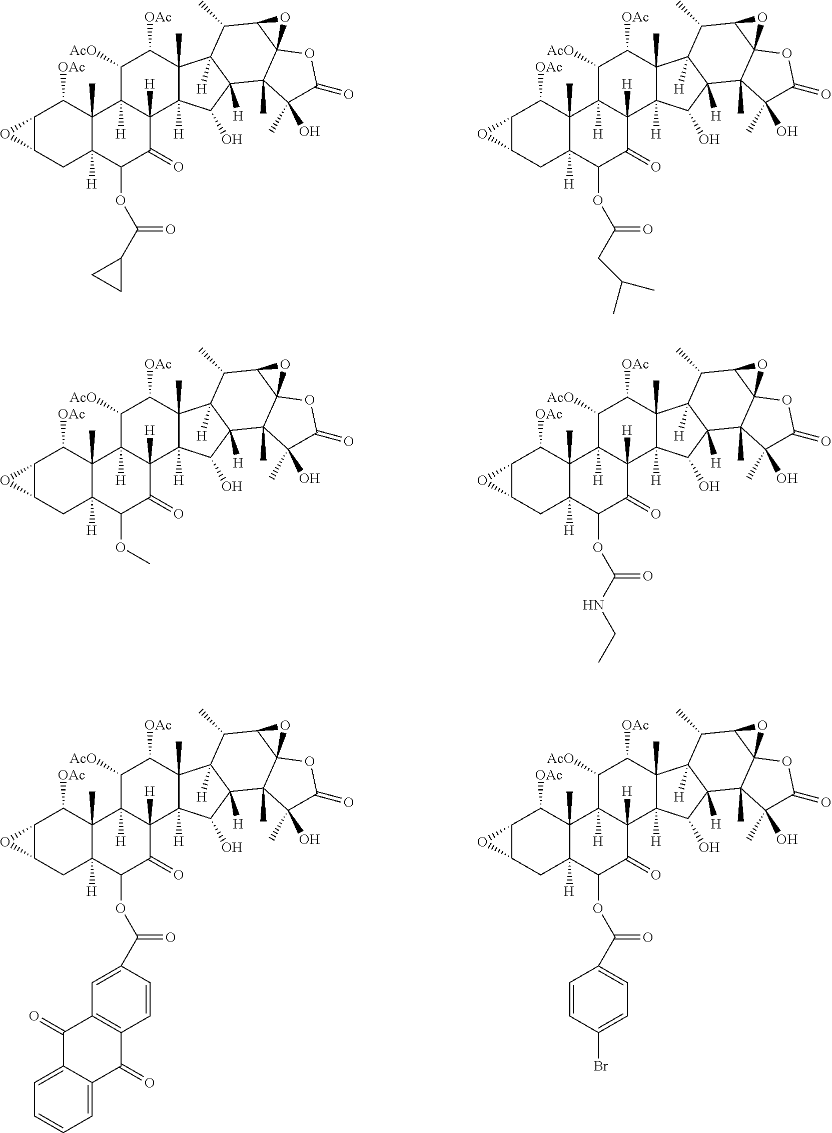

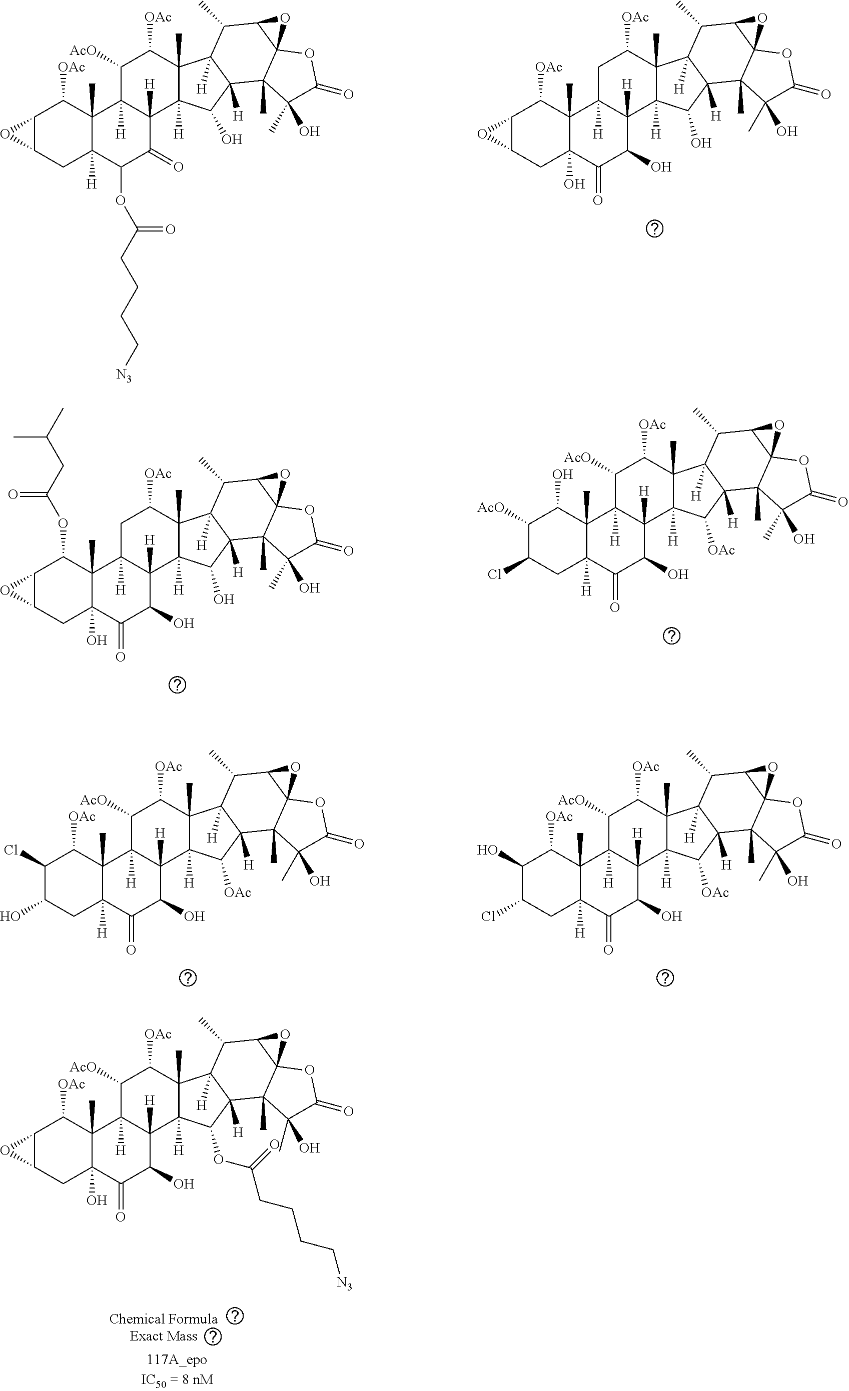

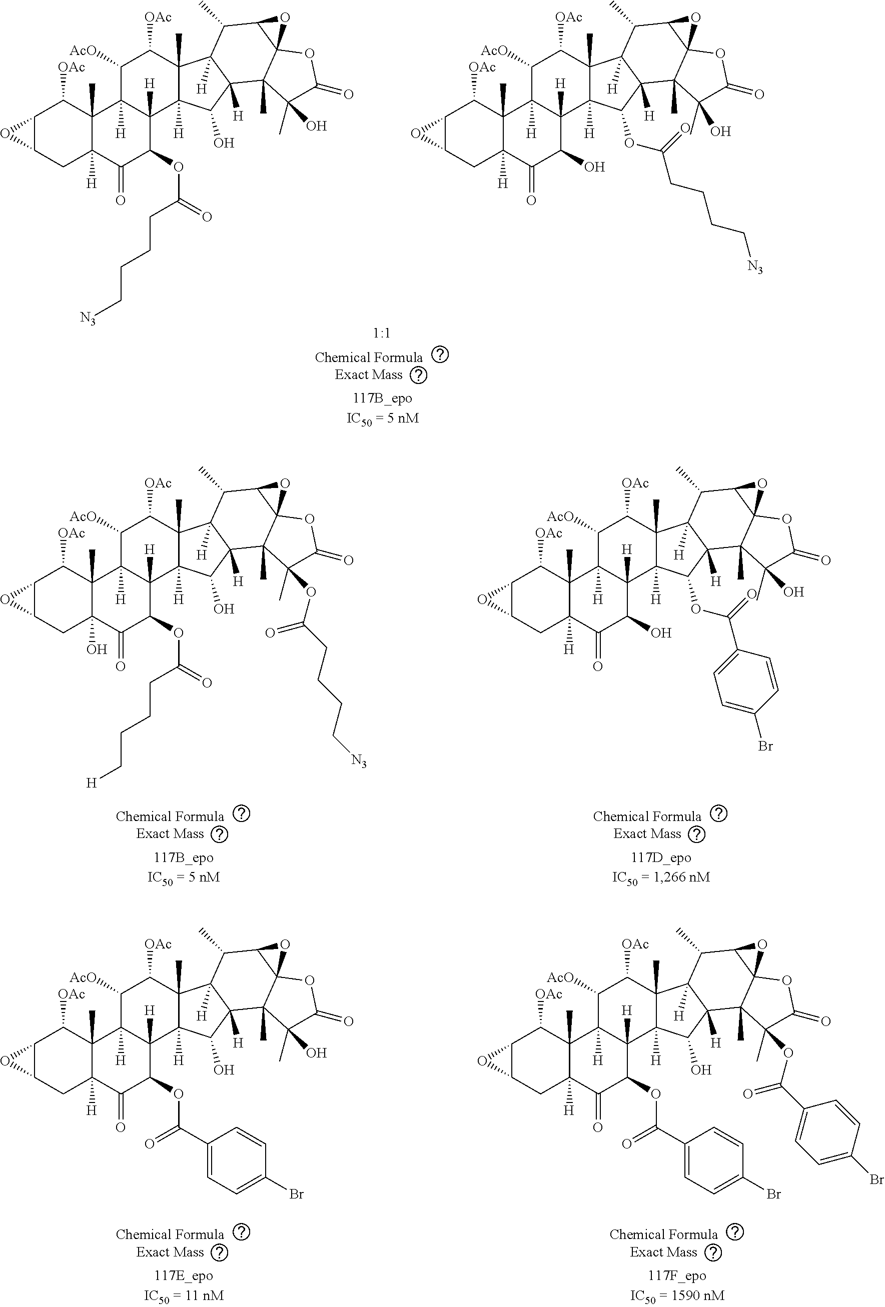

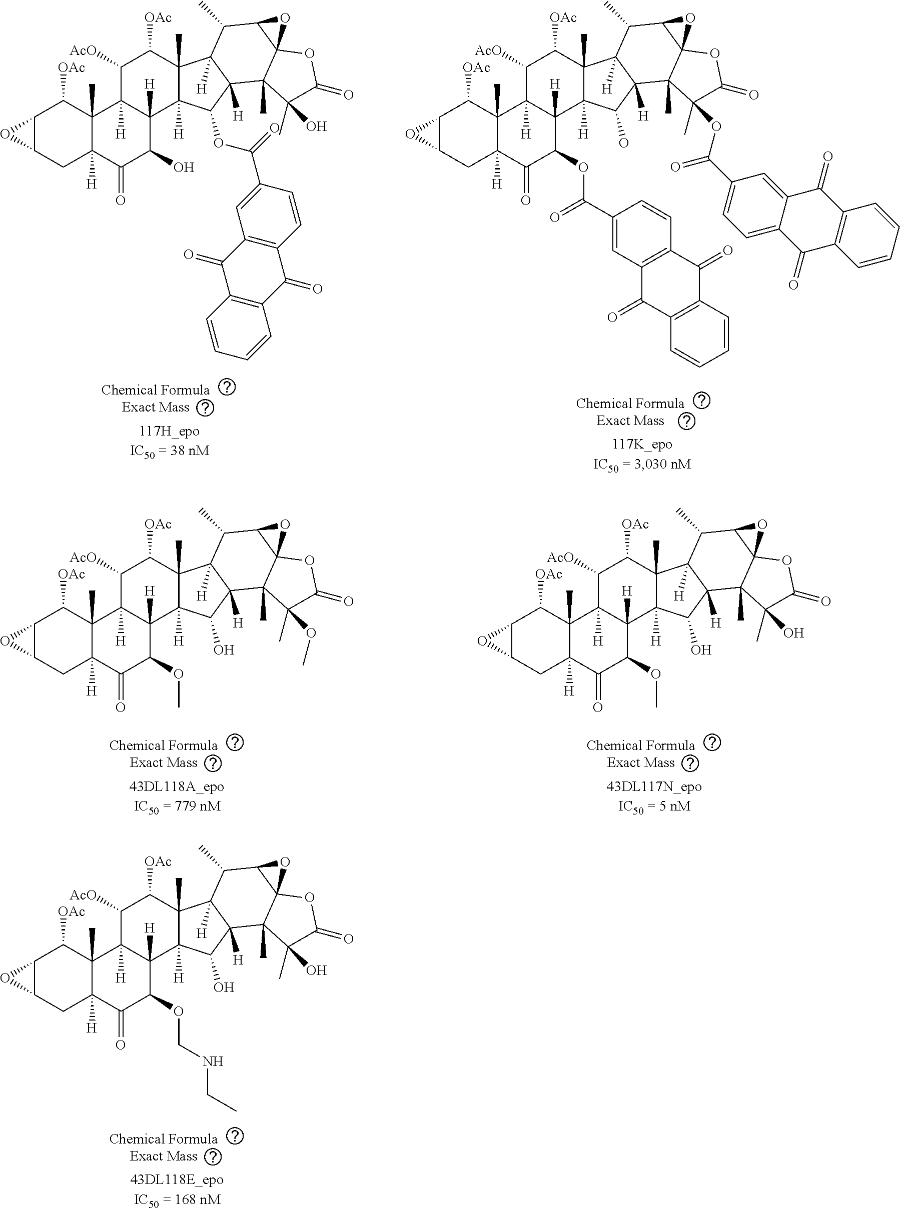

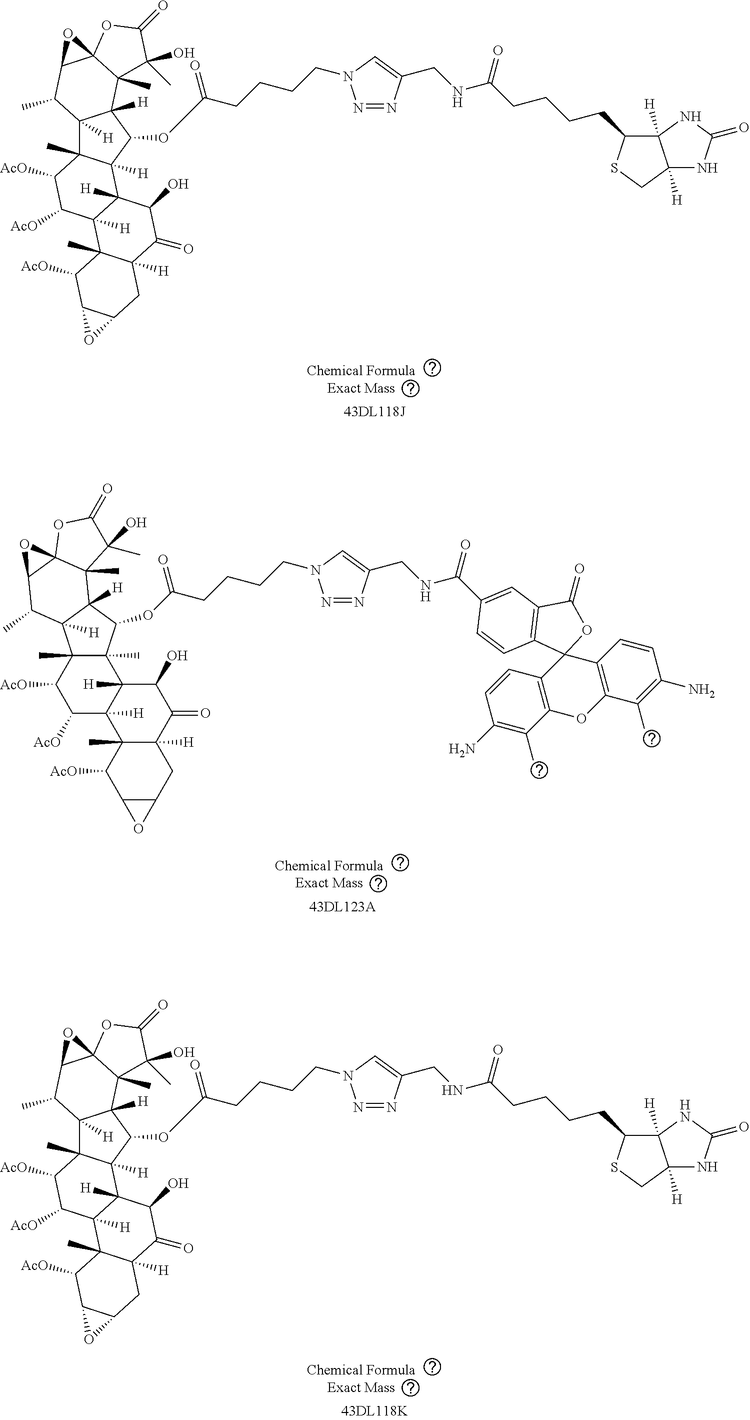

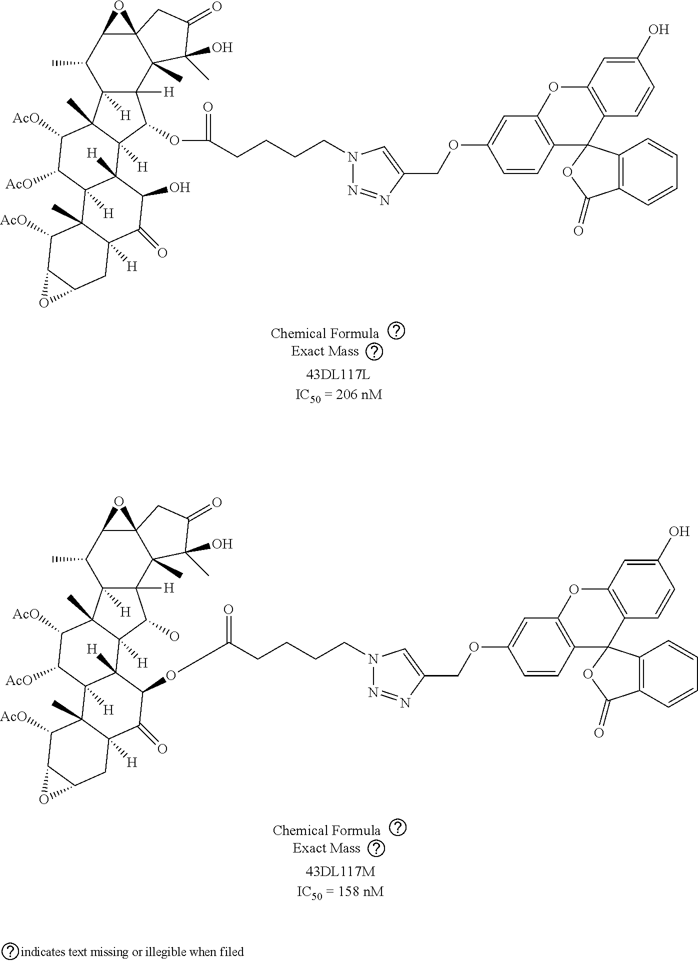

In a further aspect, the compound is at least 90% pure by weight. In a further aspect, the compound is at least 95% pure by weight. In a further aspect, the compound was isolated from plant cell tissue. In a further aspect, the compound was not isolated from cell tissue.

In another aspect, there are provided pharmaceutical compositions comprising a compound disclosed herein and a pharmaceutically acceptable carrier. In a further aspect, the composition is formulated for oral administration. In a further aspect, the compositions further comprise one or more pharmaceutically acceptable excipients. In a further aspect, the composition is formulated for controlled release.

In a further aspect, there are provided compositions comprising at least 90% by weight of a disclosed compound.

In another aspect there are provided methods of treating a hyperproliferative disorder in a patient, the method comprising administering to a patient in need thereof an effective amount of a compound disclosed herein. In a further aspect, the hyperproliferative disorder is cancer. In a further aspect, the cancer is lung cancer, brain cancer, head & neck cancer, breast cancer, skin cancer, liver cancer, pancreatic cancer, prostate cancer, stomach cancer, colon cancer, rectal cancer, uterine cancer, cervical cancer, ovarian cancer, testicular cancer, skin cancer, oral cancer, or esophageal cancer. In a further aspect, the hyperproliferative disorder is leukemia, lymphoma or myeloma. In a further aspect, the hyperproliferative disorder is acute myeloid leukemia, chronic myelogenous leukemia or multiple myeloma. In a further aspect, the patient is human.

In another aspect, there are provided methods of producing a mixture of epoxytaccalonolides, the method comprising subjecting a solution of a taccalonolide-containing crude extract of the roots and/or rhizomes of a Tacca species in an organic solvent to epoxidation.

In another aspect, there are provided methods of producing a mixture of epoxytaccalonolides, the method comprising: (a) dissolving a taccalonide-containing a crude extract of the roots and/or rhizomes of a Tacca species in an organic solvent; and (b) subjecting the solution of (a) to epoxidation. In a further aspect, the Tacca species is T. chantrieri, T. integrifolia, T. plantaginea, T. pinnatifida leontopetaloides or T. cristata aspera. In a further aspect, the organic solvent is CH₂Cl₂, CH₃Cl, ethylacetate, dimethyl ether, acetone, methanol, ethanol or isopropanol. In a further aspect, the solution of step (a) is maintained at about −70 to about 40° C. In a further aspect, tep (b) comprises contacting the solution of step (a) with dimethyldioxirane, peracide or hydroperoxide at about −70 to about 70° C. until complete. In a further aspect, wherein step (b) comprises contacting the solution of step (a) with about 1 to about 10 equivalents of 0.01-0.2M dimethyldioxirane. In a further aspect, further comprising evaporating the solvents and reagents of step (b) to isolate said epoxytaccalonolides.

In another aspect, there are provided uses of a disclosed compound in the preparation of a medicament for the treatment of a hyperproliferative disorder in a patient.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed.

FIG. 1 shows representative structures of the taccalonolides AF, AJ, and AI.

FIG. 2A-D shows representative data illustrating the effect of the taccalonolides on interphase cells.

FIG. 3A-D shows representative data illustrating the effect of the taccalonolides on cell cycle distribution.

FIG. 4A-D shows representative data illustrating the effect of the taccalonolides on mitotic spindles.

FIG. 5 shows representative data illustrating the effect of the taccalonolides on purified porcine brain tubulin.

FIG. 6 shows representative antitumor activity of taccalonolide AF as compared to paclitaxel in a triple-negative breast tumor, MDA-MB-231.

FIG. 7A and FIG. 7B collectively present a representative comparison of the DFT-calculated ¹³C NMR chemical shifts of two 22,23-isomers of taccalonolide AF.

FIG. 8A and FIG. 8B show representative data illustrating the acidic hydrolysis of 22, 23-epoxide and the absolute configuration of the hydrolytic product.

FIG. 9 presents a ¹H NMR (DMSO-d₆, 25° C.) spectrum of compound 1.

FIG. 10 presents a ¹³C NMR (DMSO-d₆, 25° C.) spectrum of compound 1.

FIG. 11 presents a ¹H-¹H COSY (DMSO-d₆, 25° C.) spectrum of compound 1.

FIG. 12 presents a HSQC (DMSO-d₆, 25° C.) spectrum of compound 1.

FIG. 13 presents a HMBC (DMSO-d₆, 25° C.) spectrum of compound 1.

FIG. 14A-C shows a representative semisynthesis and biological effects of C-6 modified taccalonolides.

FIG. 15A-D show representative data of the effects of taccalonolide AF on the growth of breast cancer cells in the brain as compared to paclitaxel.

FIG. 16 shows representative antitumor activity of taccalonolide AF as compared to paclitaxel in a multi-drug resistant ovarian tumor, NCI/ADR-RES

### DETAILED DESCRIPTION

The taccalonolides are a unique class of microtubule stabilizers with activity against drug resistant cells in vitro and in vivo. In the work described below, the inventors generated by isolation and semi-synthesis new taccalonolides including taccalonolides AF, AJ and AI-epo.

Taccalonolide structures were determined by 1D and 2D NMR methods. Each of these taccalonolides stabilizes cellular microtubules, causing the formation of microtubule bundles and mitotic accumulation of cancer cells with multiple abnormal mitotic spindles. IC₅₀ values range from the low nanomolar range for taccalonolide AI-epo (0.73 nM) and taccalonolide AJ (4.3 nM) to the low micromolar range for taccalonolide R (13 μM). These studies demonstrate that diverse taccalonolides possess microtubule stabilizing properties and that significant structure-activity relationships exist. These and other aspects of the invention are discussed further below.

The taccalonolides are a class of structurally and mechanistically distinct microtubule-stabilizing agents isolated from Tacca chantrieri. An important feature of the taxane family of microtubule stabilizers is their susceptibility to cellular resistance mechanisms including overexpression of P-glycoprotein (Pgp), multidrug resistance protein 7 (MRP7), and the βIII isotype of tubulin.

The compounds provided by the present disclosure are shown above in the summary of the invention section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In a further aspect, a single diastereomer is obtained. The chiral centers of the compounds of the present invention can have the S or the R configuration, as defined by the IUPAC 1974 Recommendations. For example, mixtures of stereoisomers may be separated using the techniques taught in the Examples section below, as well as modifications thereof.

Atoms making up the compounds of the present invention are intended to include all isotopic forms of such atoms. Compounds of the present invention include those with one or more atoms that have been isotopically modified or enriched, in particular those with pharmaceutically acceptable isotopes or those useful for pharmaceutical research. Isotopes, as used herein, include those atoms having the same atomic number but different mass numbers. By way of general example and without limitation, isotopes of hydrogen include deuterium and tritium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It should be further recognized that the compounds of the present invention include those that have been further modified to comprise substituents that are convertible to hydrogen in vivo. This includes those groups that may be convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxy carbonyl group include ethoxy carbonyl, tert-butoxy carbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), lie (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxy carbonyl groups (such as benzyloxy carbonyl and p-nitrobenzyloxy carbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

Compounds of the invention may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

The compound may be a mixture of epoxytaccalonolides (defined as a taccalonolide with 1 C22,23-epoxyl group), which contains two or more multiple compounds in any ratio with structures represented by the above formulae. The mixture of epoxytaccalonolides may be produced by epoxidation of a crude extract of the roots and/or rhizomes of the Tacca species, including but not limited to, T. chantrieri, T. integrifolia, T. plantaginea, T. pinnatifida leontopetaloides, and T. cristata aspera.

The hyperproliferative cell may be a solid tumor cancer cell, such as a lung cancer cell, a brain cancer cell, a head and neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a prostate cancer cell, a skin cancer cell, an oral cancer cell or a esophageal cancer cell. The cancer cell may alternatively be a leukemia, lymphoma, or myeloma cell, such as an acute myeloid leukemia, chronic myelogenous leukemia or multiple myeloma. The hyperproliferative mammalian cell might be an endothelial or smooth muscle cell that lines blood vessels or a cell of the skin such as an epidermal cell or melanocyte.

The hyperproliferating cell may be located in a subject, such as a human subject. The method may then further comprising administering to said subject a second therapy, such as chemotherapy, radiotherapy, immunotherapy, toxin therapy, hormone therapy, gene therapy or surgery. The second therapy may be given at the same time as said compound, or before or after said compound.

The present invention also provides a mixture of epoxytaccalonolides (defined as a taccalonolide with a C22,23-epoxyl group), which contains two or more compounds in any ratio with structures represented by the above formulae. The mixture of epoxytaccalonolides may be produced by epoxidation of a crude extract of the roots and/or rhizomes of the Tacca species, including but not limited to, T. chantrieri, T. integrifolia, T. plantaginea, T. pinnatifida leontopetaloides, and T. cristata aspera.

### A. DEFINITIONS

Listed below are definitions of various terms used to describe this invention. These definitions apply to the terms as they are used throughout this specification, unless otherwise limited in specific instances, either individually or as part of a larger group.

When used in the context of a chemical group, “hydrogen” means —H; “hydroxy” means —OH; “hydroperoxy” means —OOH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, for example, the structure

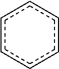

includes the structures

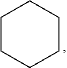

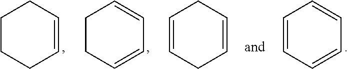

As will be understood by a person of skill in the art, no one such ring atom forms part of more than one double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation (e.g., either R or S) or the geometry is undefined (e.g., either E or Z).

Any undefined valency on an atom of a structure shown in this application implicitly represents a hydrogen atom bonded to the atom. When a group “R” is depicted as a “floating group” on a ring system, for example, in the formula:

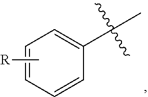

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

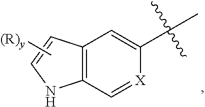

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the following parenthetical subscripts further define the group/class as follows: “(Cn)” defines the exact number (n) of carbon atoms in the group/class. “(C≤n)” defines the maximum number (n) of carbon atoms that can be in the group/class, with the minimum number as small as possible for the group in question, e.g., it is understood that the minimum number of carbon atoms in the group “alkenyl_{(C≤8)}” or the class “alkene_{(C≤8)}” is two. For example, “alkoxy_{(C≤10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “saturated” as used herein means the compound or group so modified has no carbon-carbon double and no carbon-carbon triple bonds, except as noted below. The term does not preclude carbon-heteroatom multiple bonds, for example a carbon oxygen double bond or a carbon nitrogen double bond. Moreover, it does not preclude a carbon-carbon double bond that may occur as part of keto-enol tautomerism or imine/enamine tautomerism.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl). When the term “aliphatic” is used without the “substituted” modifier only carbon and hydrogen atoms are present. When the term is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “alkyl” when used without the “substituted” modifier refers to a monovalent saturated aliphatic group with a carbon atom as the point of attachment, a linear or branched, cyclo, cyclic or acyclic structure, and no atoms other than carbon and hydrogen. Thus, as used herein cycloalkyl is a subset of alkyl. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

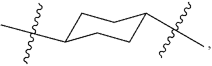 [SMILES: *C1CCC(*)CC1]

are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which one or more hydrogen atoms has been substituted with a halo group and no other atoms aside from carbon, hydrogen and halogen are present. The group, —CH₂C1 is a non-limiting examples of a haloalkyl. An “alkane” refers to the compound H—R, wherein R is alkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups. An “alkane” refers to the compound H—R, wherein R is alkyl.

The term “alkenyl” when used without the “substituted” modifier refers to an monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

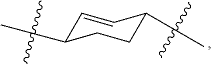 [SMILES: *C1C=CC(*)CC1]

are non-limiting examples of alkenediyl groups. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —CH═CHF, —CH═CHCl and —CH═CHBr, are non-limiting examples of substituted alkenyl groups. An “alkene” refers to the compound H—R, wherein R is alkenyl.

The term “alkynyl” when used without the “substituted” modifier refers to an monovalent unsaturated aliphatic group with a carbon atom as the point of attachment, a linear or branched, cyclo, cyclic or acyclic structure, at least one carbon-carbon triple bond, and no atoms other than carbon and hydrogen. As used herein, the term alkynyl does not preclude the presence of one or more non-aromatic carbon-carbon double bonds. The groups, —C≡CH, —C≡CCH₃, and —CH₂C≡CCH₃, are non-limiting examples of alkynyl groups. When alkynyl is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “alkyne” refers to the compound H—R, wherein R is alkynyl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl group (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and the monovalent group derived from biphenyl. The term “arenediyl” when used without the “substituted” modifier refers to a divalent aromatic group, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl group (carbon number limitation permitting) attached to the first aromatic ring or any additional aromatic ring present. If more than one ring is present, the rings may be fused or unfused. Non-limiting examples of arenediyl groups include:

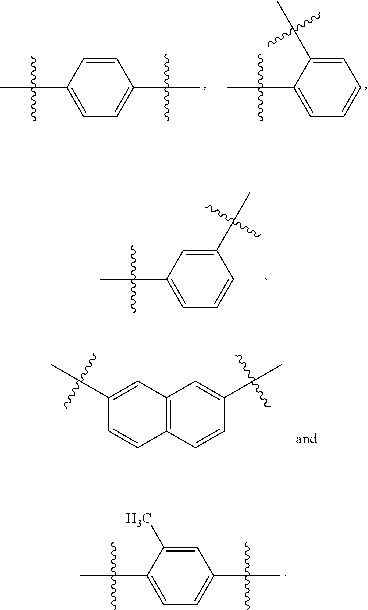

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. An “arene” refers to the compound H—R, wherein R is aryl.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn) and 2-phenyl-ethyl. When the term is used with the “substituted” modifier one or more hydrogen atom from the alkanediyl and/or the aryl has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, said atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. As used herein, the term does not preclude the presence of one or more alkyl, aryl, and/or aralkyl groups (carbon number limitation permitting) attached to the aromatic ring or aromatic ring system. If more than one ring is present, the rings may be fused or unfused. Non-limiting examples of heteroarenediyl groups include:

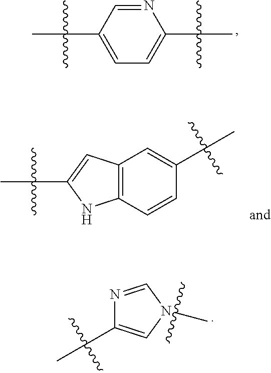

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. As used herein, the term does not preclude the presence of one or more alkyl groups (carbon number limitation permitting) attached to the ring or ring system. If more than one ring is present, the rings may be fused or unfused. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, and pyranyl. When the term “heterocycloalkyl” used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂.

The term “acyl” when used without the “substituted” modifier refers to the group —C(O)R, in which R is a hydrogen, alkyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C6H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. When either of these terms are used with the “substituted” modifier one or more hydrogen atom (including the hydrogen atom directly attached the carbonyl or thiocarbonyl group) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples of alkoxy groups include: —OCH₃ (methoxy), —OCH₂CH₃ (ethoxy), —OCH₂CH₂CH₃, —OCH(CH₃)₂ (isopropoxy), —OCH(CH₂)₂, —O-cyclopentyl, and —O-cyclohexyl. The terms “alkenyloxy”, “alkynyloxy”, “aryloxy”, “aralkoxy”, “heteroaryloxy”, and “acyloxy”, when used without the “substituted” modifier, refers to groups, defined as —OR, in which R is alkenyl, alkynyl, aryl, aralkyl, heteroaryl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The term “alcohol” corresponds to an alkane, as defined above, wherein at least one of the hydrogen atoms has been replaced with a hydroxy group.

The term “alkylamino” when used without the “substituted” modifier refers to the group —NHR, in which R is an alkyl, as that term is defined above. Non-limiting examples of alkylamino groups include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “alkoxyamino”, “alkenylamino”, “alkynylamino”, “arylamino”, “aralkylamino”, “heteroarylamino”, and “alkylsulfonylamino” when used without the “substituted” modifier, refers to groups, defined as —NHR, in which R is alkoxy, alkenyl, alkynyl, aryl, aralkyl, heteroaryl, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. The term “alkylaminodiyl” refers to the divalent group —NH-alkanediyl-, —NH-alkanediyl-NH—, or -alkanediyl-NH-alkanediyl-. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

As used herein, a “chiral auxiliary” refers to a removable chiral group that is capable of influencing the stereoselectivity of a reaction. Persons of skill in the art are familiar with such compounds, and many are commercially available.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### B. COMPOUNDS

In one aspect, disclosed are compounds useful in treating or preventing a hyperproliferative disorder. In a further aspect, the disclosed compounds cause microtubule disruption. In a still further aspect, the disclosed compounds exhibit inhibition of microtubule-dependent processes.

In one aspect, the compounds of the invention are useful in the treatment or prevention of hyperproliferative disorders and other diseases in which microtubules are involved, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

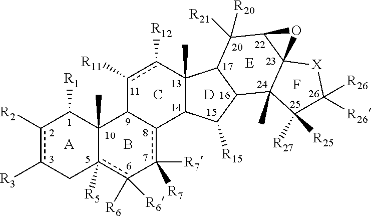

wherein: R₁ is hydroxy, alkoxy_{(C≤12)} or acyloxy_{(C≤12)}; R₂ is hydroxy, halogen, or R₂ is taken together with R₃ to form an epoxide at C-2/C-3; R₃ is hydroxy, halo, or R₂ is taken together with R₃ as defined above; R₅ is hydrogen, hydroxy, amino, alkoxy_{(C≤9)}, alkylamino_{(C≤6)}, or dialkylamino_{(C≤12)}; R₆ is hydrogen, hydroxy, alkoxy_{(C≤30)}, acyloxy_{(C≤30)}, or oxo if R_{6′} is not present; R_{6′} when present is hydrogen or hydroxy, alkoxy_{(C≤30)} or acyloxy_{(C≤30)}; R₇ is hydrogen, hydroxy, alkoxy_{(C≤30)}, acyloxy_{(C≤30)}, or oxo if R_{7′} is not present; R_{7′} when present is hydrogen, hydroxy, alkoxy_{(C≤30)}, or acyloxy_{(C≤30)}; R₁₁ is hydrogen, hydroxy, alkyl_{(C≤6)}, alkoxy_{(C≤8)}, or acyloxy_{(C≤8)}; R₁₂ is hydrogen, hydroxy, alkyl_{(C≤6)}, alkoxy_{(C≤8)}, or acyloxy_{(C≤8)}; R₁₅ is hydrogen, hydroxy, alkyl_{(C≤30)}, alkoxy_{(C≤30)} or acyloxy_{(C≤30)}; R₂₀ is hydrogen, hydroxy, hydroperoxy, alkoxy_{(C≤8)} or acyloxy_{(C≤8)}; R₂₁ is hydrogen or alkyl_{(C≤6)}; R₂₅ is hydrogen, hydroxy, alkoxy_{(C≤8)} or acyloxy_{(C≤8)}; R₂₆ is hydrogen, hydroxy, alkoxy_{(C≤8)} or oxo if R_{26′} is not present; R_{26′} when present is hydrogen, hydroxy or alkoxy_{(C≤8)}; R₂₇ is hydrogen or alkyl_{(C≤6)}; and X is O, NR^{x} or CR^{x}₂, wherein each R^{x} is independently hydrogen or alkyl_{(C≤6)}; or a pharmaceutically acceptable salt thereof.

In one aspect, disclosed are compounds having a structure represented by a formula:

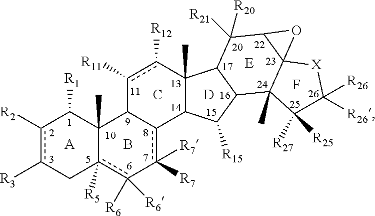

wherein each --- is an optional covalent bond; wherein R₁ is selected from —OH, C1-C12 hydroxy, C1-C12 alkoxy, and —OC(O)(C1-C12 alkyl); wherein each of R₂ and R₃ is independently selected from hydrogen, —OH, C1-C12 hydroxy, and halogen, or wherein R₂ and R₃ together comprise —O—; wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent; wherein each of R₆ and R_{6′} is independently selected from hydrogen, —OH, C1-C30 hydroxy, C1-C30 alkoxy, C1-C30 acyloxy, —OC(O)Ar₁, —OC(O)Ar₂, —OC(O)(C1-C4 alkyl)Ar₂, and —OC(O)(C1-C8 azide); wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; or wherein each of R₆ and R_{6′} together comprise ═O, or wherein one of R₆ and R_{6′} is absent; wherein each of R₇ and R_{7′} is independently selected from hydrogen, —OH, C1-C30 hydroxy, C1-C30 alkoxy, and C1-C30 acyloxy, or wherein each of R₇ and R_{7′} together comprise ═O, or wherein one of R₇ and R_{7′} is absent; wherein each of R₁₁ and R₁₂ is independently selected from hydrogen, —OH, C1-C8 hydroxy, C1-C6 alkyl, C1-C8 alkoxy, and C1-C8 acyloxy; wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, and —OC(O)(C1-C8 azide); wherein each of R₃₁ₐ and R_{31b}, when present, is independently selected from hydrogen and C1-C8 alkyl; wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

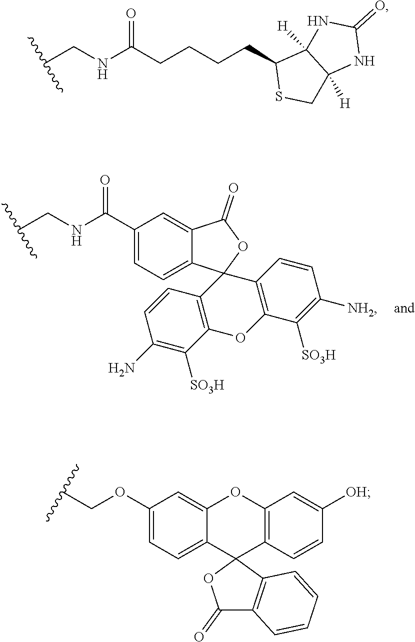

wherein R₂₀ is selected from hydrogen, —OH, —OOH, C1-C8 hydroxy, C1-C8 hydroperoxy, C1-C8 alkoxy, and C1-C8 acyloxy; wherein R₂₁ is selected from hydrogen and C1-C6 alkyl; wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar¹, and —OC(O)(C1-C8 azide); wherein each of R₂₆ and R_{26′} is independently selected from hydrogen, —OH, C1-C8 hydroxy, and C1-C8 alkoxy, or wherein each of R₂₆ and R_{26′} together comprise ═O; wherein R₂₇ is selected from hydrogen and C1-C6 alkyl; and wherein X is selected from O, NR^{x}, and CR^{x}₂; wherein R^{x}, when present, is selected from hydrogen and C1-C6 alkyl, or a pharmaceutically acceptable salt thereof.

In one aspect, disclosed are compounds having a structure represented by a formula:

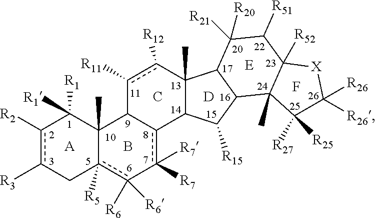

wherein each --- is an optional covalent bond; wherein R₁ is selected from —OH, C1-C12 hydroxy, C1-C12 alkoxy, —OC(O)(C1-C12 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃, and wherein R_{1′} is hydrogen; or wherein each of R₁ and R_{1′} together comprise ═O or ═NR₄₆; wherein each of R₂ and R₃ is independently selected from hydrogen, —OH, C1-C12 hydroxy, and halogen, or wherein R₂ and R₃ together comprise an epoxide at C-2/C-3; wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent; wherein each of R₆ and R_{6′} is independently selected from hydrogen, —OH, C1-C30 hydroxy, C1-C30 alkoxy, C1-C30 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃; or wherein each of R₆ and R_{6′} together comprise ═O or ═NR₄₆, or wherein one of R₆ and R_{6′} is absent; wherein R₇ is selected from hydrogen, —OH, C1-C30 hydroxy, C1-C30 alkoxy, C1-C30 acyloxy, and —OC(O)NR₃₁ₐR_{31b}, and wherein R_{7′} is selected from hydrogen, —OH, C1-C30 hydroxy, C1-C30 alkoxy, and C1-C30 acyloxy; or wherein each of R₇ and R_{7′} together comprise ═O; or wherein one of R₇ and R_{7′} is absent; wherein each of R₁₁ and R₁₂ is independently selected from hydrogen, —OH, C1-C8 hydroxy, C1-C6 alkyl, C1-C8 alkoxy, and C1-C8 acyloxy; wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, —OC(O)(C1-C8 azide), and —OC(O)CH₃; wherein R₂₀ is selected from hydrogen, —OH, —OOH, C1-C8 hydroxy, C1-C8 hydroperoxy, C1-C8 alkoxy, and C1-C8 acyloxy; wherein R₂₁ is selected from hydrogen and C1-C6 alkyl; wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar₁, and —OC(O)(C1-C8 azide); wherein each of R₂₆ and R_{26′} is independently selected from hydrogen, —OH, C1-C8 hydroxy, and C1-C8 alkoxy, or wherein each of R₂₆ and R_{26′} together comprise ═O; wherein R₂₇ is selected from hydrogen and C1-C6 alkyl; and wherein each of R₃₁ₐ and R_{31b}, when present, is independently selected from hydrogen and C1-C12 alkyl; wherein each occurrence of R₄₁, R₄₂, R₄₄, R₄₅ₐ, and R_{45b}, when present, is independently selected from hydrogen and C1-C12 alkyl; wherein each occurrence of R₄₃, when present, is independently selected from hydrogen, C1-C12 alkyl, and monocyclic aryl monosubstituted with a methyl group; wherein each occurrence of R₄₆, when present, is independently selected from hydrogen and C1-C12 alkyl; wherein each of R₅₁ and R₅₂ is independently halogen; or wherein each of R₅₁ and R₅₂ together comprise —O— or —N(R₅₃)—; wherein R₅₃, when present, is selected from hydrogen, C1-C4 alkyl, —SO₂R₅₄, and a structure having a formula:

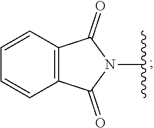

wherein R₅₄, when present, is selected from hydrogen, C1-C4 alkyl, —CH₂CH₂Si(CH₃)₃, and monocyclic aryl monosubstituted with a methyl group; wherein each occurrence of Cy¹, when present, is independently heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

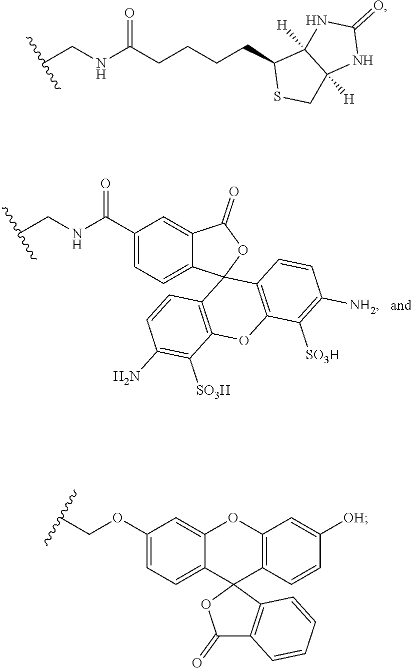

wherein each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino; wherein X is selected from O, NR^{x}, and CR^{x}₂; wherein R^{x}, when present, is selected from hydrogen and C1-C6 alkyl, or a pharmaceutically acceptable salt thereof.

In a further aspect, the compound has a structure represented by a formula:

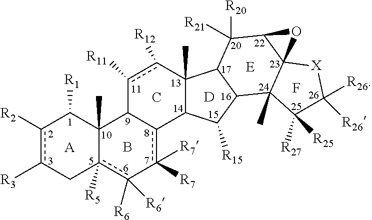

In a further aspect, the compound has a structure represented by a formula:

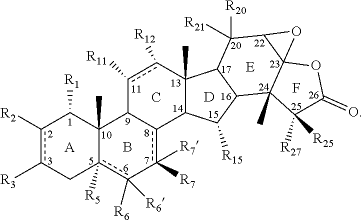

In a further aspect, the compound has a structure represented by a formula:

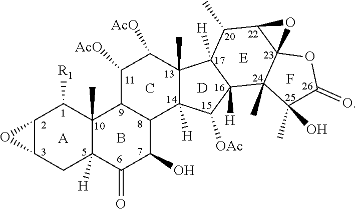

In a further aspect, the compound has a structure represented by a formula:

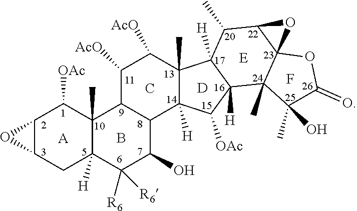

In a further aspect, the compound has a structure represented by a formula:

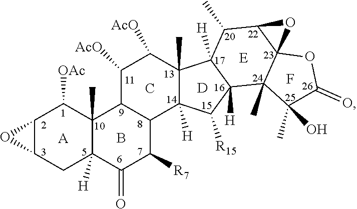

wherein R₇ is selected from —OH and —OC(O)NR^{31a}R^{31b}; and wherein R₁₅ is selected from —OH, —OC(O)NR^{31a}R^{31b}, and —OC(O)CH₃.

In a further aspect, the compound has a structure represented by a formula:

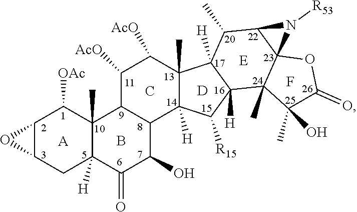

wherein R₁₅ is selected from —OH and —OC(O)CH₃; and wherein R₅₃ is selected from hydrogen, methyl, —SO₂CH₂CH₂Si(CH₃)₃, and a structure selected from:

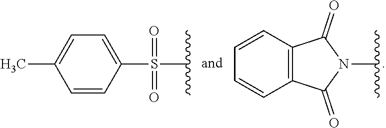 [SMILES: *S(=O)(=O)c1ccc(C)cc1 ; *N1C(=O)c2ccccc2C1=O]

In a further aspect, the compound has a structure represented by a formula:

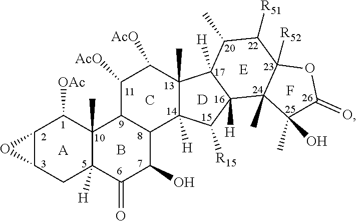

wherein R₁₅ is selected from —OH and —OC(O)CH₃; and wherein each of R₅₁ and R₅₂ is halogen.

In a further aspect, C7/C8 are connected with a double bond.

In a further aspect, R₁ is acyloxy_{(C3-12)}; In a further aspect, C7/C8 are connected with a double bond; In a further aspect, R₅ is a hydroxy or alkyl_{(C≤6)}.

a. X Groups

In one aspect, X is O, NR^{x} or CR^{x}₂. In one aspect, X is selected from O, NR^{x}, and CR^{x}₂.

In a further aspect, X is selected from O and NR^{x}. In a still further aspect, X is selected from O and CR^{x}₂. In yet a further aspect, X is selected from NR^{x} and CR^{x}₂. In an even further aspect, X is O. In a still further aspect, X is NR^{x}. In yet a further aspect, X is CR^{x}₂.

b. R₁ and R_{1′} Groups

In one aspect, R₁ is hydroxy, alkoxy_{(C≤12)} or acyloxy_{(C≤12)}.

In one aspect, R₁ is selected from —OH, C1-C12 hydroxy, C1-C12 alkoxy, and —OC(O)(C1-C12 alkyl). In a further aspect, R₁ is selected from —OH, C1-C8 hydroxy, C1-C8 alkoxy, and —OC(O)(C1-C8 alkyl). In a still further aspect, R₁ is selected from —OH, C1-C4 hydroxy, C1-C4 alkoxy, and —OC(O)(C1-C4 alkyl). In yet a further aspect, R₁ is selected from —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R₁ is selected from —OH, —CH₂OH, —CH₂CH₂OH, —OCH₃, —OCH₂CH₃, —OC(O)CH₃, and —OC(O)CH₂CH₃. In a still further aspect, R₁ is selected from —OH, —CH₂OH, —OCH₃, and —OC(O)CH₃.

In one aspect, R₁ is selected from —OH, C1-C12 hydroxy, C1-C12 alkoxy, —OC(O)(C1-C12 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃, and R₁ is hydrogen; or each of R₁ and R_{1′} together comprise ═O or ═NR₄₆.

In a further aspect, R₁ is selected from —OH, C1-C12 hydroxy, C1-C12 alkoxy, —OC(O)(C1-C12 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃. In a still further aspect, R₁ is selected from —OH, C1-C8 hydroxy, C1-C8 alkoxy, —OC(O)(C1-C8 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C8 alkyl, C2-C8 alkenyl, C2-C8 alkynyl, C1-C8 thioalkyl, C1-C8 alkylthio, C1-C8 aminoalkyl, C1-C8 alkylamino, (C1-C8)(C1-C8) dialkylamino, —OP(O)(OR₄₂)₂, —OSO₂R₄₃, —C(O)(C1-C8 alkyl), —CO₂R₄₄, —C(O)NR₄₅ₐR_{45b}, —(C1-C8 alkyl)C(O)NR₄₅ₐR_{45b}, —OC(O)NR₄₅ₐR_{45b}, —(C1-C8 alkyl)OC(O)NR₄₅ₐR_{45b}, Cy₁, Ar₃, (C1-C8 alkyl)Ar₃, and —OAr₃. In yet a further aspect, R₁ is selected from —OH, C1-C4 hydroxy, C1-C4 alkoxy, —OC(O)(C1-C4 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C4 alkyl, C2-C4 alkenyl, C2-C4 alkynyl, C1-C4 thioalkyl, C1-C4 alkylthio, C1-C4 aminoalkyl, C1-C4 alkylamino, (C1-C4)(C1-C4) dialkylamino, —OP(O)(OR₄₂)₂, —OSO₂R₄₃, —C(O)(C1-C4 alkyl), —CO₂R₄₄, —C(O)NR₄₅ₐR_{45b}, —(C1-C4 alkyl)C(O)NR₄₅ₐR_{45b}, —OC(O)NR₄₅ₐR_{45b}, —(C1-C4 alkyl)OC(O)NR₄₅ₐR_{45b}, Cy₁, Ar₃, (C1-C4 alkyl)Ar₃, and —OAr₃.

In a further aspect, each of R₁ and R_{1′} together comprise ═O or ═NR₄₆. In a still further aspect, each of R₁ and R_{1′} together comprise ═O. In yet a further aspect, each of R₁ and R_{1′} together comprise ═NR₄₆.

In a further aspect, R₁ is acyloxy_{(C≤12)}. In a further aspect, R₁ is acetyloxy. In a further aspect, R₁ is acyloxy_{(C3-12)}. In a further aspect, R₁ is hydroxy.

In a further aspect, R₁ is selected from —OH, C1-C12 alkoxy, and C1-C12 acyloxy. In a still further aspect, R₁ is selected from —OH, C1-C8 alkoxy, and C1-C8 acyloxy. In yet a further aspect, R₁ is selected from —OH, C1-C4 alkoxy, and C1-C4 acyloxy. In an even further aspect, R₁ is selected from —OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In a still further aspect, R₁ is selected from —OH, —OCH₃, —OCH₂CH₃, —OC(O)CH₃, and —OC(O)CH₂CH₃. In yet a further aspect, R₁ is selected from —OH, —OCH₃, and —OC(O)CH₃.

In a further aspect, R₁ is selected from —OH and C1-C12 acyloxy. In a still further aspect, R₁ is selected from —OH and C1-C8 acyloxy. In yet a further aspect, R₁ is selected from —OH and C1-C4 acyloxy. In an even further aspect, R₁ is selected from —OH, —OC(O)CH₃, —OC(O)CH₂CH₃, —OC(O)CH(CH₃)₂, and —OC(O)CH₂CH₂CH₃. In a still further aspect, R₁ is selected from —OH, —OC(O)CH₃, and —OC(O)CH₂CH₃. In yet a further aspect, R₁ is selected from —OH and —OC(O)CH₃.

In a further aspect, R₁ is selected from —OH and C1-C12 alkoxy. In a still further aspect, R₁ is selected from —OH and C1-C8 alkoxy. In yet a further aspect, R₁ is selected from —OH and C1-C4 alkoxy. In an even further aspect, R₁ is selected from —OH, —OCH₃, —OCH₂CH₃, —OCH(CH₃)₂, and —OCH₂CH₂CH₃. In a still further aspect, R₁ is selected from —OH, —OCH₃, and —OCH₂CH₃, —OC(O)CH₃. In yet a further aspect, R₁ is selected from —OH and —OCH₃.

In a further aspect, R₁ is C1-C12 acyloxy. In a still further aspect, R₁ is C1-C8 acyloxy. In yet a further aspect, R₁ is C1-C4 acyloxy. In an even further aspect, R₁ is selected from —OC(O)CH₃, —OC(O)CH₂CH₃, —OC(O)CH(CH₃)₂, and —OC(O)CH₂CH₂CH₃. In a still further aspect, R₁ is selected from —OC(O)CH₃ and —OC(O)CH₂CH₃. In yet a further aspect, R₁ is —OC(O)CH₃.

In a further aspect, R₁ is C1-C12 alkoxy. In a still further aspect, R₁ is C1-C8 alkoxy. In yet a further aspect, R₁ is C1-C4 alkoxy. In an even further aspect, R₁ is selected from —OCH₃, —OCH₂CH₃, —OCH(CH₃)₂, —OCH₂CH₂CH₃. In a still further aspect, R₁ is selected from —OCH₃ and —OCH₂CH₃. In yet a further aspect, R₁ is —OCH₃.

In a further aspect, R₁ is —OH.

c. R₂ and R₃ Groups

In one aspect, R₂ is hydroxy, halogen, or R₂ is taken together with R₃ to form an epoxide at C-2/C-3 and R₃ is hydroxy, halo, or R₂ is taken together with R₃ as defined above.

In one aspect, each of R₂ and R₃ is independently selected from hydrogen, —OH, C1-C12 hydroxy, and halogen, or wherein R₂ and R₃ together comprise —O—.

In a further aspect, R₂ is acyloxy_{(C≤12)}. In a further aspect, R₂ is acetyloxy. In a further aspect, R₂ and R₃ are taken together to form an epoxide at C-2/C-3. In a further aspect, R₃ is chloro.

In a further aspect, each of R₂ and R₃ is independently selected from hydrogen, —OH, C1-C12 hydroxy, and halogen. In a still further aspect, each of R₂ and R₃ is independently selected from hydrogen, —OH, C1-C8 hydroxy, and halogen. In yet a further aspect, each of R₂ and R₃ is independently selected from hydrogen, —OH, C1-C4 hydroxy, and halogen. In an even further aspect, each of R₂ and R₃ is independently selected from hydrogen, —OH, —CH₂OH, —CH₂CH₂OH, —CH(CH₃)CH₂OH, —CH₂CH₂CH₂OH, and halogen. In a still further aspect, each of R₂ and R₃ is independently selected from hydrogen, —OH, —CH₂OH, —CH₂CH₂OH, and halogen. In yet a further aspect, each of R₂ and R₃ is independently selected from hydrogen, —OH, —CH₂OH, and halogen.

In a further aspect, each of R₂ and R₃ is independently selected from —OH and halogen. In a still further aspect, each of R₂ and R₃ is independently selected from —OH, —F, and —Cl. In yet a further aspect, each of R₂ and R₃ is independently selected from —OH and —Cl. In an even further aspect, each of R₂ and R₃ is independently selected from —OH and —F.

In a further aspect, each of R₂ and R₃ is —OH.

In a further aspect, each of R₂ and R₃ is independently halogen. In a still further aspect, each of R₂ and R₃ is independently selected from —F and —Cl. In yet a further aspect, each of R₂ and R₃ is —Cl. In an even further aspect, each of R₂ and R₃ is —F.

In a further aspect, R₂ and R₃ are taken together to form an epoxide. In a further aspect, R₂ and R₃ together comprise —O—.

d. R₅ Groups

In one aspect, R₅ is hydrogen, hydroxy, amino, alkoxy_{(C≤9)}, alkylamino_{(C≤6)}, or dialkylamino_{(C≤12)}.

In one aspect, R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent.

In a further aspect, R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino. In a still further aspect, R₅ is selected from hydrogen, —OH, —NH₂, C1-C6 alkyl, C1-C8 hydroxy, C1-C8 aminoalkyl, C1-C8 alkoxy, C1-C8 alkylamino, and (C1-C6)(C1-C6) dialkylamino. In yet a further aspect, R₅ is selected from hydrogen, —OH, —NH₂, C1-C4 alkyl, C1-C4 hydroxy, C1-C4 aminoalkyl, C1-C4 alkoxy, C1-C4 alkylamino, and (C1-C4)(C1-C4) dialkylamino. In an even further aspect, R₅ is selected from hydrogen, —OH, —NH₂, methyl, ethyl, n-propyl, i-propyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₂CH₃)₂, —N(CH₃)(CH(CH₃)₂), —N(CH₃)(CH₂CH₂CH₃), and —N(CH₃)(CH₂CH₃). In a still further aspect, R₅ is selected from hydrogen, —OH, —NH₂, methyl, ethyl, —CH₂OH, —CH₂CH₂OH, —OCH₃, —OCH₂CH₃, —NHCH₃, —NHCH₂CH₃, —N(CH₃)₂, and —N(CH₂CH₃)₂. In yet a further aspect, R₅ is selected from hydrogen, —OH, —NH₂, methyl, —CH₂OH, —OCH₃, —NHCH₃, and —N(CH₃)₂.

In a further aspect, R₅ is absent.

In a further aspect, R₅ is hydrogen. In a still further aspect, R₅ is hydroxy. In yet a further aspect, R₅ is absent. In an even further aspect, R₅ is a hydroxy or alkyl_{(C≤6)}.

In a further aspect, R₅ is selected from hydrogen, —OH, —NH₂, C1-C9 alkoxy, C1-C6 alkylamino, and (C1-C6)(C1-C6) dialkylamino. In a still further aspect, R₅ is selected from hydrogen, —OH, —NH₂, C1-C8 alkoxy, C1-C6 alkylamino, and (C1-C6)(C1-C6) dialkylamino. In yet a further aspect, R₅ is selected from hydrogen, —OH, —NH₂, C1-C4 alkoxy, C1-C4 alkylamino, and (C1-C4)(C1-C4) dialkylamino. In an even further aspect, R₅ is selected from hydrogen, —OH, —NH₂, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₂CH₃)₂, —N(CH₃)(CH(CH₃)₂), —N(CH₃)(CH₂CH₂CH₃), and —N(CH₃)(CH₂CH₃). In a still further aspect, R₅ is selected from hydrogen, —OH, —NH₂, —OCH₃, —OCH₂CH₃, —NHCH₃, —NHCH₂CH₃, —N(CH₃)₂, —N(CH₂CH₃)₂, and —N(CH₃)(CH₂CH₃). In yet a further aspect, R₅ is selected from hydrogen, —OH, —NH₂, —OCH₃, —NHCH₃, and —N(CH₃)₂.

In a further aspect, R₅ is selected from hydrogen, —OH, —NH₂, and C1-C9 alkoxy. In a still further aspect, R₅ is selected from hydrogen, —OH, —NH₂, and C1-C8 alkoxy. In yet a further aspect, R₅ is selected from hydrogen, —OH, —NH₂, and C1-C4 alkoxy. In an even further aspect, R₅ is selected from hydrogen, —OH, —NH₂, —OCH₃, —OCH₂CH₃, —OCH(CH₃)₂, and —OCH₂CH₂CH₃. In a still further aspect, R₅ is selected from hydrogen, —OH, —NH₂, —OCH₃, and —OCH₂CH₃. In yet a further aspect, R₅ is selected from hydrogen, —OH, —NH₂, and —OCH₃.

In a further aspect, R₅ is selected from hydrogen, —OH, —NH₂, C1-C6 alkylamino, and (C1-C6)(C1-C6) dialkylamino. In yet a further aspect, R₅ is selected from hydrogen, —OH, —NH₂, C1-C4 alkylamino, and (C1-C4)(C1-C4) dialkylamino. In an even further aspect, R₅ is selected from hydrogen, —OH, —NH₂, —NHCH₃, —NHCH₂CH₃, —NHCH(CH₃)₂, —NHCH₂CH₂CH₃, —N(CH₃)₂, —N(CH₂CH₃)₂, —N(CH₃)(CH(CH₃)₂), —N(CH₃)(CH₂CH₂CH₃), and —N(CH₃)(CH₂CH₃). In a still further aspect, R₅ is selected from hydrogen, —OH, —NH₂, —NHCH₃, —NHCH₂CH₃, —N(CH₃)₂, —N(CH₂CH₃)₂, and —N(CH₃)(CH₂CH₃). In yet a further aspect, R₅ is selected from hydrogen, —OH, —NH₂, —NHCH₃, and —N(CH₃)₂.

In a further aspect, R₅ is selected from hydrogen, —OH, and —NH₂. In a still further aspect, R₅ is selected from hydrogen and —OH. In a still further aspect, R₅ is selected from hydrogen and —NH₂. In yet a further aspect, R₅ is hydrogen. In an even further aspect R₅ is —OH. In a still further aspect, R₅ is —NH₂.

In a further aspect, R₅ is selected from C1-C6 alkylamino and (C1-C6)(C1-C6) dialkylamino. In yet a further aspect, R₅ is selected from C1-C4 alkylamino and (C1-C4)(C1-C4) dialkylamino. In an even further aspect, R₅ is selected from —NHCH₃, —NHCH₂CH₃, —NHCH(CH₃)₂, —NHCH₂CH₂CH₃, —N(CH₃)₂, —N(CH₂CH₃)₂, —N(CH₃)(CH(CH₃)₂), —N(CH₃)(CH₂CH₂CH₃), and —N(CH₃)(CH₂CH₃). In a still further aspect, R₅ is selected from —NHCH₃, —NHCH₂CH₃, —N(CH₃)₂, —N(CH₂CH₃)₂, and —N(CH₃)(CH₂CH₃). In yet a further aspect, R₅ is selected from —NHCH₃ and —N(CH₃)₂.

In a further aspect, R₅ is C1-C9 alkoxy. In a still further aspect, R₅ is C1-C8 alkoxy. In yet a further aspect, R₅ is C1-C4 alkoxy. In an even further aspect, R₅ is selected from —OCH₃, —OCH₂CH₃, —OCH(CH₃)₂, and —OCH₂CH₂CH₃. In a still further aspect, R₅ is selected from —OCH₃ and —OCH₂CH₃. In yet a further aspect, R₅ is —OCH₃.

e. R₆ and R_{6′} Groups

In one aspect, R₆ is hydrogen, hydroxy, alkoxy_{(C≤30)}, acyloxy_{(C≤30)}, or oxo if R_{6′} is not present and R_{6′} when present is hydrogen or hydroxy, alkoxy_{(C≤30)} or acyloxy_{(C≤30)}.

In one aspect, each of R₆ and R_{6′} is independently selected from hydrogen, —OH, C1-C30 hydroxy, C1-C30 alkoxy, C1-C30 acyloxy, —OC(O)Ar₁, and —OC(O)(C1-C8 azide), or wherein each of R₆ and R_{6′} together comprise ═O, or one of R₆ and R_{6′} is absent.

In one aspect, each of R₆ and R_{6′} is independently selected from hydrogen, —OH, C1-C30 hydroxy, C1-C30 alkoxy, C1-C30 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C1-C12 alkenyl, C1-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃; or each of R₆ and R_{6′} together comprise ═O or ═NR₄₆; or one of R₆ and R_{6′} is absent.

In a further aspect, each of R₆ and R_{6′} is independently selected from hydrogen, —OH, C1-C30 hydroxy, C1-C30 alkoxy, C1-C30 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C1-C12 alkenyl, C1-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃. In a still further aspect, each of R₆ and R_{6′} is independently selected from hydrogen, —OH, C1-C15 hydroxy, C1-C15 alkoxy, C1-C15 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C1-C12 alkenyl, C1-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃. In yet a further aspect, each of R₆ and R_{6′} is independently selected from hydrogen, —OH, C1-C8 hydroxy, C1-C8 alkoxy, C1-C8 acyloxy, —OC(O)Ar₁, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C8 alkyl, C1-C8 alkenyl, C1-C8 alkynyl, C1-C8 thioalkyl, C1-C8 alkylthio, C1-C8 aminoalkyl, C1-C8 alkylamino, (C1-C8)(C1-C8) dialkylamino, —OP(O)(OR⁴²)₂, —OSO₂R⁴³, —C(O)(C1-C8 alkyl), —CO₂R₄₄, —C(O)NR₄₅ₐR_{45b}, —(C1-C8 alkyl)C(O)NR₄₅ₐR_{45b}, —OC(O)NR₄₅ₐR_{45b}, —(C1-C8 alkyl)OC(O)NR₄₅ₐR_{45b}, Cy₁, Ar₃, (C1-C8 alkyl)Ar₃, and —OAr₃. In an even further aspect, each of R₆ and R_{6′} is independently selected from hydrogen, —OH, C1-C4 hydroxy, C1-C4 alkoxy, C1-C4 acyloxy, —OC(O)Ar₁, —OC(O)(C1-C4 azide), halogen, —CN, —NC, —NCO, —OCN, —NO₂, —ONO₂, —ONO, —NO, —N₃, —NH₂, —NH₃, —N═NR₄₁, —NHOH, C1-C4 alkyl, C1-C4 alkenyl, C1-C4 alkynyl, C1-C4 thioalkyl, C1-C4 alkylthio, C1-C4 aminoalkyl, C1-C4 alkylamino, (C1-C4)(C1-C4) dialkylamino, —OP(O)(OR⁴²)₂, —OSO₂R⁴³, —C(O)(C1-C4 alkyl), —CO₂R₄₄, —C(O)NR₄₅ₐR_{45b}, —(C1-C4 alkyl)C(O)NR₄₅ₐR_{45b}, —OC(O)NR₄₅ₐR_{45b}, —(C1-C4 alkyl)OC(O)NR₄₅ₐR_{45b}, Cy₁, Ar₃, (C1-C4 alkyl)Ar₃, and —OAr₃.

In a further aspect, each of R₆ and R_{6′} together comprise ═O or ═NR₄₆. In a still further aspect, each of R₆ and R_{6′}, together comprise ═O. In yet a further aspect, each of R₆ and R_{6′} together comprise ═NR₄₆.

In a further aspect, each of R₆ and R_{6′} is independently selected from hydrogen, —OH, C1-C30 hydroxy, C1-C30 alkoxy, C1-C30 acyloxy, —OC(O)Ar₁, and —OC(O)(C1-C8 azide). In a further aspect, each of R₆ and R_{6′} is independently selected from hydrogen, —OH, C1-C15 hydroxy, C1-C15 alkoxy, C1-C15 acyloxy, —OC(O)Ar₁, and —OC(O)(C1-C8 azide). In a still further aspect, each of R₆ and R_{6′} is independently selected from hydrogen, —OH, C1-C8 hydroxy, C1-C8 alkoxy, C1-C8 acyloxy, —OC(O)Ar₁, and —OC(O)(C1-C8 azide). In yet a further aspect, each of R₆ and R_{6′} is independently selected from hydrogen, —OH, C1-C4 hydroxy, C1-C4 alkoxy, C1-C4 acyloxy, —OC(O)Ar₁, and —OC(O)(C1-C4 azide). In an even further aspect, each of R₆ and R_{6′} is independently selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, —OC(O)Ar₁, —OC(O)CH₂N₃, —OC(O)CH₂CH₂N₃, —OC(O)CH(CH₃)CH₂N₃, and —OC(O)CH₂CH₂CH₂N₃. In a still further aspect, each of R₆ and R_{6′} is independently selected from hydrogen, —OH, —CH₂OH, —CH₂CH₂OH, —OCH₃, —OCH₂CH₃, —OC(O)CH₃, —OC(O)CH₂CH₃, —OC(O)Ar₁, —OC(O)CH₂N₃, and —OC(O)CH₂CH₂N₃. In yet a further aspect, each of R₆ and R_{6′} is independently selected from hydrogen, —OH, —CH₂OH, —OCH₃, —OC(O)CH₃, —OC(O)Ar₁, and —OC(O)CH₂N₃.

In a further aspect, one of R₆ and R_{6′} is absent.

In a further aspect, R₆ is oxo. In a further aspect, R₆ is hydroxy. In a further aspect, R₆ is acyloxy_{(C1-30)}. In a further aspect, R₆ is acyloxy_{(C1-24)}. In a further aspect, R₆ is acyloxy_{(C1-18)}. In a further aspect, R₆ is acyloxy_{(C1-12)}. In a further aspect, R₆ is acyloxy_{(C1-8)}. In a further aspect, R₆ is acetyloxy. In a further aspect, R₆ and R₇ are taken together to form an epoxide at C-6/C-7. In a further aspect, R_{6′} is absent.

In a further aspect, R_{6′} is hydrogen. In a further aspect, R_{6′} is hydroxy. In a further aspect, R_{6′} is alkoxy_{(C1-30)}. In a further aspect, R_{6′} is alkoxy₍₁₋₂₄₎. In a further aspect, R_{6′} is alkoxy₍₁₋₁₈₎. In a further aspect, R_{6′} is alkoxy₍₁₋₁₂₎. In a further aspect, R_{6′} is alkoxy₍₁₋₈₎. In a further aspect, R_{6′} is acyloxy₍₁₋₃₀₎. In a further aspect, R_{6′} is acyloxy₍₁₋₂₄₎. In a further aspect, R_{6′} is acyloxy₍₁₋₁₈₎. In a further aspect, R_{6′} is acyloxy₍₁₋₁₂₎. In a further aspect, R_{6′} is acyloxy₍₁₋₈₎.

In a further aspect, R₆ and R_{6′} together comprise oxo. In a still further aspect, each of R₆ and R_{6′} together comprise ═O.

In a further aspect, each of R₆ and R_{6′} is independently selected from hydrogen, —OH, C1-C30 alkoxy, and C1-C30 acyloxy. In a still further aspect, each of R₆ and R_{6′} is independently selected from hydrogen, —OH, C1-C15 alkoxy, and C1-C15 acyloxy. In yet a further aspect, each of R₆ and R_{6′} is independently selected from hydrogen, —OH, C1-C8 alkoxy, and C1-C8 acyloxy. In an even further aspect, each of R₆ and R_{6′} is independently selected from hydrogen, —OH, C1-C4 alkoxy, and C1-C4 acyloxy. In a still further aspect, each of R₆ and R_{6′} is independently selected from hydrogen, —OH, C1-C30 alkoxy, and C1—C30 acyloxy. In yet a further aspect, each of R₆ and R_{6′} is independently selected from hydrogen, —OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, each of R₆ and R_{6′} is independently selected from hydrogen, —OH, —OCH₃, —OCH₂CH₃, —OC(O)CH₃, and —OC(O)CH₂CH₃. In a still further aspect, each of R₆ and R_{6′} is independently selected from hydrogen, —OH, —OCH₃, and —OC(O)CH₃.

In a further aspect, each of R₆ and R_{6′} is independently selected from hydrogen and —OH. In a still further aspect, each of R₆ and R_{6′} is —OH. In yet a further aspect, each of R₆ and R_{6′} is hydrogen.

In a further aspect, each of R₆ and R_{6′} is independently selected from hydrogen, —OH, and C1-C30 acyloxy. In a still further aspect, each of R₆ and R_{6′} is independently selected from hydrogen, —OH, and C1-C15 acyloxy. In yet a further aspect, each of R₆ and R_{6′} is independently selected from hydrogen, —OH, and C1-C8 acyloxy. In an even further aspect, each of R₆ and R_{6′} is independently selected from hydrogen, —OH, and C1-C4 acyloxy. In yet a further aspect, each of R₆ and R_{6′} is independently selected from hydrogen, —OH, —OC(O)CH₃, —OC(O)CH₂CH₃, —OC(O)CH(CH₃)₂, and —OC(O)CH₂CH₂CH₃. In an even further aspect, each of R₆ and R_{6′} is independently selected from hydrogen, —OH, —OC(O)CH₃, and —OC(O)CH₂CH₃. In a still further aspect, each of R₆ and R_{6′} is independently selected from hydrogen, —OH, and —OC(O)CH₃.

In a further aspect, each of R₆ and R_{6′} is independently selected from hydrogen, —OH, and C1-C30 alkoxy. In a still further aspect, each of R₆ and R_{6′} is independently selected from hydrogen, —OH, and C1-C15 alkoxy. In yet a further aspect, each of R₆ and R_{6′}, is independently selected from hydrogen, —OH, and C1-C8 alkoxy. In an even further aspect, each of R₆ and R_{6′} is independently selected from hydrogen, —OH, and C1-C4 alkoxy. In yet a further aspect, each of R₆ and R_{6′} is independently selected from hydrogen, —OH, —OCH₃, —OCH₂CH₃, —OCH(CH₃)₂, and —OCH₂CH₂CH₃. In an even further aspect, each of R₆ and R_{6′} is independently selected from hydrogen, —OH, —OCH₃, and —OCH₂CH₃. In a still further aspect, each of R₆ and R_{6′} is independently selected from hydrogen, —OH, and —OCH₃.

f. R₇ and R_{7′} Groups

In one aspect, R₇ is hydrogen, hydroxy, alkoxy_{(C≤30)}, acyloxy_{(C≤30)}, or oxo if R_{7′} is not present and R_{7′} when present is hydrogen, hydroxy, alkoxy_{(C≤30)}, or acyloxy_{(C≤30)}.

In one aspect, each of R₇ and R_{7′} is independently selected from hydrogen, —OH, C1-C30 hydroxy, C1-C30 alkoxy, and C1-C30 acyloxy, or wherein each of R₇ and R_{7′} together comprise ═O, or wherein one of R₇ and R_{7′} is absent.

In one aspect, R₇ is selected from hydrogen, —OH, C1-C30 hydroxy, C1-C30 alkoxy, C1-C30 acyloxy, and —OC(O)NR₃₁ₐR_{31b}, and R_{7′} is selected from hydrogen, —OH, C1-C30 hydroxy, C1-C30 alkoxy, and C1-C30 acyloxy; or each of R₇ and R_{7′} together comprise ═O; or one of R₇ and R_{7′} is absent.

In a further aspect, R₇ is selected from hydrogen, —OH, C1-C30 hydroxy, C1-C30 alkoxy, C1-C30 acyloxy, and —OC(O)NR₃₁ₐR_{31b}, and R_{7′} is selected from hydrogen, —OH, C1-C30 hydroxy, C1-C30 alkoxy, and C1-C30 acyloxy. In a still further aspect, R₇ is selected from hydrogen, —OH, C1-C15 hydroxy, C1-C15 alkoxy, C1-C15 acyloxy, and —OC(O)NR₃₁ₐR_{31b}, and R₇ is selected from hydrogen, —OH, C1-C15 hydroxy, C1-C15 alkoxy, and C1-C15 acyloxy. In yet a further aspect, R₇ is selected from hydrogen, —OH, C1-C8 hydroxy, C1-C8 alkoxy, C1-C8 acyloxy, and —OC(O)NR₃₁ₐR_{31b}, and R_{7′} is selected from hydrogen, —OH, C1-C8 hydroxy, C1-C8 alkoxy, and C1-C8 acyloxy. In an even further aspect, R₇ is selected from hydrogen, —OH, C1-C8 hydroxy, C1-C8 alkoxy, C1-C8 acyloxy, and —OC(O)NR₃₁ₐR_{31b}, and R_{7′} is selected from hydrogen, —OH, C1-C4 hydroxy, C1-C4 alkoxy, and C1-C4 acyloxy.

In a further aspect, each of R₇ and R_{7′} is independently selected from hydrogen, —OH, C1-C30 hydroxy, C1-C30 alkoxy, and C1-C30 acyloxy. In a still further aspect, each of R₇ and R_{7′} is independently selected from hydrogen, —OH, C1-C15 hydroxy, C1-C15 alkoxy, and C1-C15 acyloxy. In yet a further aspect, each of R₇ and R_{7′} is independently selected from hydrogen, —OH, C1-C8 hydroxy, C1-C8 alkoxy, and C1-C8 acyloxy. In an even further aspect, each of R₇ and R_{7′} is independently selected from hydrogen, —OH, C1-C4 hydroxy, C1-C4 alkoxy, and C1-C4 acyloxy. In a still further aspect, each of R₇ and R_{7′} is independently selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In yet a further aspect, each of R₇ and R_{7′} is independently selected from hydrogen, —OH, —CH₂OH, —CH₂CH₂OH, —OCH₃, —OCH₂CH₃, —OC(O)CH₃, and —OC(O)CH₂CH₃. In an even further aspect, each of R₇ and R_{7′} is independently selected from hydrogen, —OH, —CH₂OH, —OCH₃, and —OC(O)CH₃.

In a further aspect, one of R₇ and R_{7′} is absent.

In a further aspect, R₇ is acyloxy₍₁₋₃₀₎. In a further aspect, R₇ is acyloxy₍₁₋₃₀₎. In a further aspect, R₇ is acyloxy₍₁₋₂₄₎. In a further aspect, R₇ is acyloxy₍₁₋₁₈₎. In a further aspect, R₇ is acyloxy₍₁₋₁₂₎. In a further aspect, R₇ is acyloxy₍₁₋₈₎. In a further aspect, R₇ is acetyloxy. In a further aspect, R₇ is hydroxy. In a further aspect, R₇ is oxo.

In a further aspect, R_{7′} is hydrogen. In a further aspect, R_{7′} is hydroxy. In a further aspect, R_{7′} is alkoxy₍₁₋₃₀₎, In a further aspect, R_{7′} is alkoxy₍₁₋₂₄₎, In a further aspect, R_{7′} is alkoxy₍₁₋₁₈₎, In a further aspect, R_{7′} is alkoxy₍₁₋₁₂₎, In a further aspect, R_{7′} is alkoxy₍₁₋₈₎, In a further aspect, R_{7′} is acyloxy₍₁₋₃₀₎. In a further aspect, R_{7′} is acyloxy₍₁₋₂₄₎. In a further aspect, R_{7′} is acyloxy₍₁₋₁₈₎. In a further aspect, R_{7′} is acyloxy₍₁₋₁₂₎. In a further aspect, R_{7′} is acyloxy₍₁₋₈₎.

In a further aspect, R₇ and R_{7′} together comprise oxo. In a still further aspect, each of R₇ and R_{7′} together comprise ═O.

In a further aspect, each of R₇ and R_{7′} is independently selected from hydrogen, —OH, C1-C30 alkoxy, and C1-C30 acyloxy. In a still further aspect, each of R₇ and R_{7′} is independently selected from hydrogen, —OH, C1-C15 alkoxy, and C1-C15 acyloxy. In yet a further aspect, each of R₇ and R_{7′} is independently selected from hydrogen, —OH, C1-C8 alkoxy, and C1-C8 acyloxy. In an even further aspect, each of R₇ and R_{7′} is independently selected from hydrogen, —OH, C1-C4 alkoxy, and C1-C4 acyloxy. In a still further aspect, each of R₇ and R_{7′} is independently selected from hydrogen, —OH, C1-C30 alkoxy, and C1-C30 acyloxy. In yet a further aspect, each of R₇ and R_{7′} is independently selected from hydrogen, —OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, each of R₇ and R_{7′} is independently selected from hydrogen, —OH, —OCH₃, —OCH₂CH₃, —OC(O)CH₃, and —OC(O)CH₂CH₃. In a still further aspect, each of R₇ and R_{7′} is independently selected from hydrogen, —OH, —OCH₃, and —OC(O)CH₃.

In a further aspect, each of R₇ and R_{7′} is independently selected from hydrogen and —OH. In a still further aspect, each of R₇ and R_{7′} is —OH. In yet a further aspect, each of R₇ and R_{7′} is hydrogen.

In a further aspect, each of R₇ and R_{7′} is independently selected from hydrogen, —OH, and C1-C30 acyloxy. In a still further aspect, each of R₇ and R_{7′} is independently selected from hydrogen, —OH, and C1-C15 acyloxy. In yet a further aspect, each of R₇ and R_{7′} is independently selected from hydrogen, —OH, and C1-C8 acyloxy. In an even further aspect, each of R₇ and R_{7′} is independently selected from hydrogen, —OH, and C1-C4 acyloxy. In yet a further aspect, each of R₇ and R_{7′} is independently selected from hydrogen, —OH, —OC(O)CH₃, —OC(O)CH₂CH₃, —OC(O)CH(CH₃)₂, and —OC(O)CH₂CH₂CH₃. In an even further aspect, each of R₇ and R_{7′} is independently selected from hydrogen, —OH, —OC(O)CH₃, and —OC(O)CH₂CH₃. In a still further aspect, each of R₇ and R_{7′} is independently selected from hydrogen, —OH, and —OC(O)CH₃.

In a further aspect, each of R₇ and R_{7′} is independently selected from hydrogen, —OH, and C1-C30 alkoxy. In a still further aspect, each of R₇ and R_{7′} is independently selected from hydrogen, —OH, and C1-C15 alkoxy. In yet a further aspect, each of R₇ and R_{7′} is independently selected from hydrogen, —OH, and C1-C8 alkoxy. In an even further aspect, each of R₇ and R_{7′} is independently selected from hydrogen, —OH, and C1-C4 alkoxy. In yet a further aspect, each of R₇ and R_{7′} is independently selected from hydrogen, —OH, —OCH₃, —OCH₂CH₃, —OCH(CH₃)₂, and —OCH₂CH₂CH₃. In an even further aspect, each of R₇ and R_{7′} is independently selected from hydrogen, —OH, —OCH₃, and —OCH₂CH₃. In a still further aspect, each of R₇ and R_{7′} is independently selected from hydrogen, —OH, and —OCH₃.

g. R₁₁ and R₁₂ Groups

In one aspect, R₁₁ is hydrogen, hydroxy, alkyl_{(C≤6)}, alkoxy_{(C≤8)}, or acyloxy_{(C≤8)}.

In one aspect, R₁₂ is hydrogen, hydroxy, alkyl_{(C≤6)}, alkoxy_{(C≤8)}, or acyloxy_{(C≤8)}.

In one aspect, each of R₁₁ and R₁₂ is independently selected from hydrogen, —OH, C1-C8 hydroxy, C1-C6 alkyl, C1-C8 alkoxy, and C1-C8 acyloxy. In a further aspect, each of R₁₁ and R₁₂ is independently selected from hydrogen, —OH, C1-C4 hydroxy, C1-C4 alkyl, C1-C4 alkoxy, and C1-C4 acyloxy. In a still further aspect, each of R₁₁ and R₁₂ is independently selected from hydrogen, —OH, methyl, ethyl, n-propy, i-propyl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In yet a further aspect, each of R₁₁ and R₁₂ is independently selected from hydrogen, —OH, methyl, ethyl, —CH₂OH, —CH₂CH₂OH, —OCH₃, —OCH₂CH₃, —OC(O)CH₃, and —OC(O)CH₂CH₃. In an even further aspect, each of R₁₁ and R₁₂ is independently selected from hydrogen, —OH, methyl, —CH₂OH, —OCH₃, and —OC(O)CH₃.

In a further aspect, R₁₁ is acyloxy_{(C≤12)}. In a further aspect, R₁₁ is acetyloxy. In a further aspect, R₁₁ is hydrogen. In a further aspect, R₁₁ is substituted acyloxy_{(C≤12)}. In a further aspect, R₁₁ is hydroxy.

In a further aspect, R₁₁ is selected from hydrogen, —OH, C1-C6 alkyl, C1-C6 alkoxy, and C1-C6 acyloxy. In a still further aspect, R₁₁ is selected from hydrogen, —OH, C1-C4 alkyl, C1-C4 alkoxy, and C1-C4 acyloxy. In yet a further aspect, R₁₁ is selected from hydrogen, —OH, methyl, ethyl, n-propyl, i-propyl, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R₁₁ is selected from hydrogen, —OH, methyl, ethyl, —OCH₃, —OCH₂CH₃, —OC(O)CH₃, and —OC(O)CH₂CH₃. In a still further aspect, R₁₁ is selected from hydrogen, —OH, methyl, —OCH₃, and —OC(O)CH₃.

In a further aspect, R₁₁ is selected from hydrogen and —OH. In a still further aspect, R₁₁ is —OH. In yet a further aspect, R₁₁ is hydrogen.

In a further aspect, R₁₁ is selected from hydrogen, —OH, and C1-C6 alkyl. In a still further aspect, R₁₁ is selected from hydrogen, —OH, and C1-C4 alkyl. In yet a further aspect, R₁₁ is selected from hydrogen, —OH, methyl, ethyl, n-propyl, and i-propyl. In an even further aspect, R₁₁ is selected from hydrogen, —OH, methyl, and ethyl. In a still further aspect, R₁₁ is selected from hydrogen, —OH, and methyl.

In a further aspect, R₁₁ is selected from hydrogen, —OH, and C1-C6 alkoxy. In a still further aspect, R₁₁ is selected from hydrogen, —OH, and C1-C4 alkoxy. In yet a further aspect, R₁₁ is selected from hydrogen, —OH, —OCH₃, —OCH₂CH₃, —OCH(CH₃)₂, and —OCH₂CH₂CH₃. In an even further aspect, R₁₁ is selected from hydrogen, —OH, methyl, ethyl, —OCH₃, and —OCH₂CH₃. In a still further aspect, R₁₁ is selected from hydrogen, —OH, methyl, and —OCH₃.

In a further aspect, R₁₁ is selected from hydrogen, —OH, and C1-C6 acyloxy. In a still further aspect, R₁₁ is selected from hydrogen, —OH, and C1-C4 acyloxy. In yet a further aspect, R₁₁ is selected from hydrogen, —OH, —OC(O)CH₃, —OC(O)CH₂CH₃, —OC(O)CH(CH₃)₂, and —OC(O)CH₂CH₂CH₃. In an even further aspect, R₁₁ is selected from hydrogen, —OH, —OC(O)CH₃, and —OC(O)CH₂CH₃. In a still further aspect, R₁₁ is selected from hydrogen, —OH, and —OC(O)CH₃.

In a further aspect, R₁₂ is acyloxy_{(C≤12)}. In a further aspect, R₁₂ is acetyloxy. In a further aspect, R₁₂ is hydroxy.

In a further aspect, R₁₂ is selected from hydrogen, —OH, C1-C6 alkyl, C1-C8 alkoxy, and C1-C8 acyloxy. In a still further aspect, R₁₂ is selected from hydrogen, —OH, C1-C4 alkyl, C1-C4 alkoxy, and C1-C4 acyloxy. In yet a further aspect, R₁₂ is selected from hydrogen, —OH, methyl, ethyl, n-propyl, i-propyl, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R₁₂ is selected from hydrogen, —OH, methyl, ethyl, —OCH₃, —OCH₂CH₃, —OC(O)CH₃, and —OC(O)CH₂CH₃. In a still further aspect, R₁₂ is selected from hydrogen, —OH, methyl, —OCH₃, and —OC(O)CH₃.

In a further aspect, R₁₂ is selected from hydrogen and —OH. In a still further aspect, R₁₂ is —OH. In yet a further aspect, R₁₂ is hydrogen.

In a further aspect, R₁₂ is selected from hydrogen, —OH and C1-C6 alkyl. In a still further aspect, R₁₂ is selected from hydrogen, —OH, and C1-C4 alkyl. In yet a further aspect, R₁₂ is selected from hydrogen, —OH, methyl, ethyl, n-propyl, and i-propyl. In an even further aspect, R₁₂ is selected from hydrogen, —OH, methyl, and ethyl. In a still further aspect, R₁₂ is selected from hydrogen, —OH, and methyl.

In a further aspect, R₁₂ is selected from hydrogen, —OH, and C1-C8 alkoxy. In a still further aspect, R₁₂ is selected from hydrogen, —OH, and C1-C4 alkoxy. In yet a further aspect, R₁₂ is selected from hydrogen, —OH, —OCH₃, —OCH₂CH₃, —OCH(CH₃)₂, and —OCH₂CH₂CH₃. In an even further aspect, R₁₂ is selected from hydrogen, —OH, —OCH₃, and —OCH₂CH₃. In a still further aspect, R₁₂ is selected from hydrogen, —OH, and —OCH₃.

In a further aspect, R₁₂ is selected from hydrogen, —OH, and C1-C8 acyloxy. In a still further aspect, R₁₂ is selected from hydrogen, —OH, and C1-C4 acyloxy. In yet a further aspect, R₁₂ is selected from hydrogen, —OH, —OC(O)CH₃, —OC(O)CH₂CH₃, —OC(O)CH(CH₃)₂, and —OC(O)CH₂CH₂CH₃. In an even further aspect, R₁₂ is selected from hydrogen, —OH, —OC(O)CH₃, and —OC(O)CH₂CH₃. In a still further aspect, R₁₂ is selected from hydrogen, —OH, and —OC(O)CH₃.

h. R₁₅ Groups

In one aspect, R₁₅ is hydrogen, hydroxy, alkyl_{(C≤30)}, alkoxy_{(C≤30)} or acyloxy_{(C≤30)}.

In one aspect, R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar², —OC(O)(C1-C4 alkyl)Ar₂, and —OC(O)(C1-C8 azide). In a further aspect, R₁₅ is selected from hydrogen, —OH, C1-C15 hydroxy, C1-C15 alkyl, C1-C15 alkoxy, C1-C15 acyloxy, —OC(O)NR₃₁ₐR_{31b}, —OC(O)Ar₂, —OC(O)(C1-C4 alkyl)Ar₂, and —OC(O)(C1-C8 azide). In a still further aspect, R₁₅ is selected from hydrogen, —OH, C1-C8 hydroxy, C1-C8 alkyl, C1-C8 alkoxy, C1-C8 acyloxy, —OC(O)NR₃₁ₐR_{31b}, —OC(O)Ar₂, —OC(O)(C1-C4 alkyl)Ar₂, and —OC(O)(C1-C8 azide). In yet a further aspect, R₁₅ is selected from hydrogen, —OH, C1-C4 hydroxy, C1-C4 alkyl, C1-C4 alkoxy, C1-C4 acyloxy, —OC(O)NR₃₁ₐR_{31b}, —OC(O)Ar₂, —OC(O)(C1-C4 alkyl)Ar₂, and —OC(O)(C1-C4 azide). In an even further aspect, R₁₅ is selected from hydrogen, —OH, —CH₂OH, —CH₂CH₂OH, —CH(CH₃)CH₂OH, —CH₂CH₂CH₂OH, methyl, ethyl, n-propy, i-propyl, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, —OC(O)NHCH₃, —OC(O)NHCH₂CH₃, —OC(O)NHCH(CH₃)₂, —OC(O)NHCH₂CH₂CH₃, —OC(O)N(CH₃)₂, —OC(O)N(CH₂CH₃)₂, —OC(O)N(CH₃)(CH₂CH₃), —OC(O)Ar₂, —OC(O)CH₂Ar₂, —OC(O)CH₂CH₂Ar₂, —OC(O)CH₂CH₂CH₂Ar₂, —OC(O)CH₂N₃, —OC(O)CH₂CH₂N₃, —OC(O)CH(CH₃)CH₂N₃, and —OC(O)CH₂CH₂CH₂N₃. In a still further aspect, R₁₅ is selected from hydrogen, —OH, —CH₂OH, —CH₂CH₂OH, methyl, ethyl, —OCH₃, —OCH₂CH₃, —OC(O)CH₃, —OC(O)CH₂CH₃, —OC(O)NHCH₃, —OC(O)NHCH₂CH₃, —OC(O)N(CH₃)₂, —OC(O)N(CH₂CH₃)₂, —OC(O)N(CH₃)(CH₂CH₃), —OC(O)Ar₂, —OC(O)CH₂Ar₂, —OC(O)CH₂N₃, and —OC(O)CH₂CH₂N₃. In yet a further aspect, R₁₅ is selected from hydrogen, —OH, —CH₂OH, methyl, —OCH₃, —OC(O)CH₃, —OC(O)NHCH₃, —OC(O)N(CH₃)₂, —OC(O)Ar₂, —OC(O)CH₂Ar₂, and —OC(O)CH₂N₃.

In one aspect, R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, —OC(O)(C1-C8 azide), and —OC(O)CH₃. In a further aspect, R₁₅ is selected from hydrogen, —OH, C1-C15 hydroxy, C1-C15 alkyl, C1-C15 alkoxy, C1-C15 acyloxy, —OC(O)NR₃₁ₐR_{31b}, —OC(O)Ar₂, —OC(O)(C1-C4 alkyl)Ar₂, —OC(O)(C1-C8 azide), and —OC(O)CH₃. In a still further aspect, R₁₅ is selected from hydrogen, —OH, C1-C30 hydroxy, C1-C8 alkyl, C1-C8 alkoxy, C1-C8 acyloxy, —OC(O)NR₃₁ₐR_{31b}, —OC(O)Ar₂, —OC(O)(C1-C4 alkyl)Ar₂, —OC(O)(C1-C8 azide), and —OC(O)CH₃. In yet a further aspect, R₁₅ is selected from hydrogen, —OH, C1-C4 hydroxy, C1-C4 alkyl, C1-C4 alkoxy, C1-C4 acyloxy, —OC(O)NR₃₁ₐR_{31b}, —OC(O)Ar₂, —OC(O)(C1-C4 alkyl)Ar₂, —OC(O)(C1-C4 azide), and —OC(O)CH₃.

In a further aspect, R₁₅ is hydroxy. In a further aspect, R₁₅ is hydrogen. In a further aspect, R₁₅ is oxo. In a further aspect, R₁₅ is alkyl₍₁₋₃₀₎. In a further aspect, R₁₅ is alkyl₍₁₋₂₄₎. In a further aspect, R₁₅ is alkyl₍₁₋₁₈₎. In a further aspect, R₁₅ is alkyl₍₁₋₁₂₎. In a further aspect, R₁₅ is alkyl₍₁₋₅₎. In a further aspect, R₁₅ is alkoxy₍₁₋₃₀₎. In a further aspect, R₁₅ is alkoxy₍₁₋₂₄₎. In a further aspect, R₁₅ is alkoxy₍₁₋₁₈₎. In a further aspect, R₁₅ is alkoxy₍₁₋₁₂₎₎. In a further aspect, R₁₅ is alkoxy₍₁₋₈₎. In a further aspect, R₁₅ is acyloxy₍₁₋₃₀₎. In a further aspect, R₁₅ is acyloxy₍₁₋₂₄₎. In a further aspect, R₁₅ is acyloxy₍₁₋₁₈₎. In a further aspect, R₁₅ is acyloxy₍₁₋₁₂₎. In a further aspect, R₁₅ is acyloxy₍₁₋₈₎ In a further aspect, R₁₅ is acetyloxy.

In a further aspect, R₁₅ is selected from hydrogen, —OH, C1-C30 alkyl, C1-C30 alkoxy, and C1-C30 acyloxy. In a still further aspect, R₁₅ is selected from hydrogen, —OH, C1-C15 alkyl, C1-C15 alkoxy, and C1-C15 acyloxy. In yet a further aspect, R₁₅ is selected from hydrogen, —OH, C1-C8 alkyl, C1-C8 alkoxy, and C1-C8 acyloxy. In an even further aspect, R₁₅ is selected from hydrogen, —OH, C1-C4 alkyl, C1-C4 alkoxy, and C1-C4 acyloxy. In a still further aspect, R₁₅ is selected from hydrogen, —OH, methyl, ethyl, n-propyl, i-propyl, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In yet a further aspect, R₁₅ is selected from hydrogen, —OH, methyl, ethyl, —OCH₃, —OCH₂CH₃, —OC(O)CH₃, and —OC(O)CH₂CH₃. In an even further aspect, R₁₅ is selected from hydrogen, —OH, methyl, —OCH₃, and —OC(O)CH₃.

In a further aspect, R₁₅ is selected from hydrogen and —OH. In a still further aspect, R₁₅ is —OH. In yet a further aspect, R₁₅ is hydrogen.

In a further aspect, R₁₅ is selected from hydrogen, —OH, and C1-C30 alkyl. In a still further aspect, R₁₅ is selected from hydrogen, —OH, and C1-C15 alkyl. In yet a further aspect, R₁₅ is selected from hydrogen, —OH, and C1-C8 alkyl. In an even further aspect, R₁₅ is selected from hydrogen, —OH, and C1-C4 alkyl. In a still further aspect, R₁₅ is selected from hydrogen, —OH, methyl, ethyl, n-propyl, and i-propyl. In yet a further aspect, R₁₅ is selected from hydrogen, —OH, methyl, and ethyl. In an even further aspect, R₁₅ is selected from hydrogen, —OH, and methyl.

In a further aspect, R₁₅ is selected from hydrogen, —OH, and C1-C30 alkoxy. In a still further aspect, R₁₅ is selected from hydrogen, —OH, and C1-C15 alkoxy. In yet a further aspect, R₁₅ is selected from hydrogen, —OH, and C1-C8 alkoxy. In an even further aspect, R₁₅ is selected from hydrogen, —OH, and C1-C4 alkoxy. In a still further aspect, R₁₅ is selected from hydrogen, —OH, —OCH₃, —OCH₂CH₃, —OCH(CH₃)₂, and —OCH₂CH₂CH₃. In yet a further aspect, R₁₅ is selected from hydrogen, —OH, —OCH₃, and —OCH₂CH₃. In an even further aspect, R₁₅ is selected from hydrogen, —OH, and —OCH₃.

In a further aspect, R₁₅ is selected from hydrogen, —OH, and C1-C30 acyloxy. In a still further aspect, R₁₅ is selected from hydrogen, —OH, and C1-C15 acyloxy. In yet a further aspect, R₁₅ is selected from hydrogen, —OH, and C1-C8 acyloxy. In an even further aspect, R₁₅ is selected from hydrogen, —OH, and C1-C4 acyloxy. In a still further aspect, R₁₅ is selected from hydrogen, —OH, —OC(O)CH₃, —OC(O)CH₂CH₃, —OC(O)CH(CH₃)₂, and —OC(O)CH₂CH₂CH₃. In yet a further aspect, R₁₅ is selected from hydrogen, —OH, —OC(O)CH₃, and —OC(O)CH₂CH₃. In an even further aspect, R₁₅ is selected from hydrogen, —OH, and —OC(O)CH₃.

i. R₂₀ Groups

In one aspect, R₂₀ is hydrogen, hydroxy, hydroperoxy, alkoxy_{(C≤8)} or acyloxy_{(C≤8)}.

In one aspect, R₂₀ is selected from hydrogen, —OH, —OOH, C1-C8 hydroxy, C1-C8 hydroperoxy, C1-C8 alkoxy, and C1-C8 acyloxy. In a further aspect, R₂₀ is selected from hydrogen, —OH, —OOH, C1-C4 hydroxy, C1-C4 hydroperoxy, C1-C4 alkoxy, and C1-C4 acyloxy. In a still further aspect, R₂₀ is selected from hydrogen, —OH, —OOH, —CH₂OH, —CH₂CH₂OH, —CH(CH₃)CH₂OH, —CH₂CH₂CH₂OH, —CH₂OOH, —CH₂CH₂OOH, —CH(CH₃)CH₂OOH, —CH₂CH₂CH₂OOH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In yet a further aspect, R₂₀ is selected from hydrogen, —OH, —OOH, —CH₂OH, —CH₂CH₂OH, —CH₂OOH, —CH₂CH₂OOH, —OCH₃, —OCH₂CH₃, —OC(O)CH₃, and —OC(O)CH₂CH₃. In an even further aspect, R₂₀ is selected from hydrogen, —OH, —OOH, —CH₂OH, —CH₂OOH, —OCH₃, and —OC(O)CH₃.

In a further aspect, R₂₀ is methyl. In a further aspect, R₂₀ is hydroxy. In a further aspect, R₂₀ is hydroperoxy. In a further aspect, R₂₁ is hydrogen. In a further aspect, X is O. In a further aspect, R₂₅ is hydroxy. In a further aspect, R₂₅ is acetyloxy. In a further aspect, R₂₆ is oxo. In a further aspect, R_{26′} is absent. In a further aspect, R₂₇ is methyl. In a further aspect, C7/C8 are connected with a double bond. In a further aspect, R₅ is a hydroxy or alkyl_{(C≤6)}.

In a further aspect, R₂₀ is selected from hydrogen, —OH, —OOH, C1-C8 alkoxy, and C1-C8 acyloxy. In a still further aspect, R₂₀ is selected from hydrogen, —OH, —OOH, C1-C4 alkoxy, and C1-C4 acyloxy. In yet a further aspect, R₂₀ is selected from hydrogen, —OH, —OOH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R₂₀ is selected from hydrogen, —OH, —OOH, —OCH₃, —OCH₂CH₃, —OC(O)CH₃, and —OC(O)CH₂CH₃. In a still further aspect, R₂₀ is selected from hydrogen, —OH, —OOH, —OCH₃, and —OC(O)CH₃.

In a further aspect, R₂₀ is selected from hydrogen, —OH, and —OOH. In a still further aspect, R₂₀ is selected from hydrogen and —OH. In yet a further aspect, R₂₀ is selected from hydrogen and —OOH. In an even further aspect, R₂₀ is hydrogen. In a still further aspect, R₂₀ is —OH. In yet a further aspect, R₂₀ is —OOH.

In a further aspect, R₂₀ is selected from hydrogen, —OH, —OOH, and C1-C8 alkoxy. In a still further aspect, R₂₀ is selected from hydrogen, —OH, —OOH, and C1-C4 alkoxy. In yet a further aspect, R₂₀ is selected from hydrogen, —OH, —OOH, —OCH₃, —OCH₂CH₃, —OCH(CH₃)₂, —OCH₂CH₂CH₃, and —OC(O)CH₃. In an even further aspect, R₂₀ is selected from hydrogen, —OH, —OOH, —OCH₃, and —OCH₂CH₃. In a still further aspect, R₂₀ is selected from hydrogen, —OH, —OOH, and —OCH₃.

In a further aspect, R₂₀ is selected from hydrogen, —OH, —OOH, and C1-C8 acyloxy. In a still further aspect, R₂₀ is selected from hydrogen, —OH, —OOH, and C1-C4 acyloxy. In yet a further aspect, R₂₀ is selected from hydrogen, —OH, —OOH, —OC(O)CH₃, —OC(O)CH₂CH₃, —OC(O)CH(CH₃)₂, and —OC(O)CH₂CH₂CH₃. In an even further aspect, R₂₀ is selected from hydrogen, —OH, —OOH, —OC(O)CH₃, and —OC(O)CH₂CH₃. In a still further aspect, R₂₀ is selected from hydrogen, —OH, —OOH, and —OC(O)CH₃.

j. R₂₁ Groups

In one aspect, R₂₁ is hydrogen or alkyl_{(C≤6)}. In one aspect, R₂₁ is selected from hydrogen and C1-C6 alkyl.

In a further aspect, R₂₁ is selected from hydrogen and C1-C6 alkyl. In a still further aspect, R₂₁ is selected from hydrogen and C1-C4 alkyl. In yet a further aspect, R₂₁ is selected from hydrogen, methyl, ethyl, n-propyl, and i-propyl. In an even further aspect, R₂₁ is selected from hydrogen, methyl, and ethyl. In a still further aspect, R₂₁ is selected from hydrogen and ethyl. In yet a further aspect, R₂₁ is selected from hydrogen and methyl.

In a further aspect, R₂₁ is hydrogen.

In a further aspect, R₂₁ is C1-C6 alkyl. In a still further aspect, R₂₁ is C1-C4 alkyl. In yet a further aspect, R₂₁ is selected from methyl, ethyl, n-propyl, and i-propyl. In an even further aspect, R₂₁ is selected from methyl and ethyl. In a still further aspect, R₂₁ is ethyl. In yet a further aspect, R₂₁ is methyl.

k. R₂₅ Groups

In one aspect, R₂₅ is hydrogen, hydroxy, alkoxy_{(C≤8)} or acyloxy_{(C≤8)}.

In one aspect, R₂₅ is selected from hydrogen, —OH, C1-C8 hydroxy, C1-C8 alkoxy, C1-C8 acyloxy, —OC(O)NR₃₁ₐR_{31b}, —OC(O)Ar₁, and —OC(O)(C1-C8 azide). In a further aspect, R₂₅ is selected from hydrogen, —OH, C1-C4 hydroxy, C1-C4 alkoxy, C1-C4 acyloxy, —OC(O)NR₃₁ₐR_{31b}, —OC(O)Ar₁, and —OC(O)(C1-C4 azide). In a still further aspect, R₂₅ is selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, —OC(O)CH₂CH₂CH₃, —OC(O)NR₃₁ₐR_{31b}, —OC(O)Ar₁, —OC(O)CH₂N₃, —OC(O)CH₂CH₂N₃, —OC(O)CH(CH₃)CH₂N₃, and —OC(O)CH₂CH₂CH₂N₃. In yet a further aspect, R₂₅ is selected from hydrogen, —OH, —CH₂OH, —CH₂CH₂OH, —OCH₃, —OCH₂CH₃, —OC(O)CH₃, —OC(O)CH₂CH₃, —OC(O)NR₃₁ₐR_{31b}, —OC(O)Ar₁, —OC(O)CH₂N₃, and —OC(O)CH₂CH₂N₃. In an even further aspect, R₂₅ is selected from hydrogen, —OH, —CH₂OH, —OCH₃, —OC(O)CH₃, —OC(O)NR₃₁ₐR_{3b}, —OC(O)Ar₁, and —OC(O)CH₂N₃.

In a further aspect, R₂₅ is selected from hydrogen, —OH, C1-C8 alkoxy, and C1-C8 acyloxy. In a still further aspect, R₂₅ is selected from hydrogen, —OH, C1-C4 alkoxy, and C1-C4 acyloxy. In yet a further aspect, R₂₅ is selected from hydrogen, —OH, —OCH₃, —OCH₂CH₃, —OCH(CH₃)₂, —OCH₂CH₂CH₃, —OC(O)CH₃, —OC(O)CH₂CH₃, —OC(O)CH(CH₃)₂, and —OC(O)CH₂CH₂CH₃. In an even further aspect, R₂₅ is selected from hydrogen, —OH, —OCH₃, —OCH₂CH₃, —OC(O)CH₃, and —OC(O)CH₂CH₃. In a still further aspect, R₂₅ is selected from hydrogen, —OH, —OCH₃, and —OC(O)CH₃.

In a further aspect, R₂₅ is selected from hydrogen and —OH. In a stil further aspect, R₂₅ is —OH. In yet a further aspect, R₂₅ is hydrogen.

In a further aspect, R₂₅ is selected from hydrogen, —OH, and C1-C8 alkoxy. In a still further aspect, R₂₅ is selected from hydrogen, —OH, and C1-C4 alkoxy. In yet a further aspect, R₂₅ is selected from hydrogen, —OH, —OCH₃, —OCH₂CH₃, —OCH(CH₃)₂, and —OCH₂CH₂CH₃. In an even further aspect, R₂₅ is selected from hydrogen, —OH, —OCH₃, and —OCH₂CH₃. In a still further aspect, R₂₅ is selected from hydrogen, —OH, and —OCH₃.

In a further aspect, R₂₅ is selected from hydrogen, —OH, and C1-C8 acyloxy. In a still further aspect, R₂₅ is selected from hydrogen, —OH, and C1-C4 acyloxy. In yet a further aspect, R₂₅ is selected from hydrogen, —OH, —OC(O)CH₃, —OC(O)CH₂CH₃, —OC(O)CH(CH₃)₂, and —OC(O)CH₂CH₂CH₃. In an even further aspect, R₂₅ is selected from hydrogen, —OH, —OC(O)CH₃, and —OC(O)CH₂CH₃. In a still further aspect, R₂₅ is selected from hydrogen, —OH, and —OC(O)CH₃.

l. R₂₆ and R_{26′} Groups

In one aspect, R₂₆ is hydrogen, hydroxy, alkoxy_{(C≤8)} or oxo if R_{26′} is not present and R_{26′} when present is hydrogen, hydroxy or alkoxy_{(C≤8)}.

In a further aspect, each of R₂₆ and R_{26′} is independently selected from hydrogen, —OH, C1-C8 hydroxy, and C1-C8 alkoxy, or each of R₂₆ and R_{26′} together comprise ═O.

In a further aspect, each of R₂₆ and R_{26′} is independently selected from hydrogen, —OH, C1-C8 hydroxy, and C1-C8 alkoxy. In a still further aspect, each of R₂₆ and R_{26′} is independently selected from hydrogen, —OH, C1-C4 hydroxy, and C1-C4 alkoxy. In yet a further aspect, each of R₂₆ and R_{26′} is independently selected from hydrogen, —OH, —CH₂OH, —CH₂CH₂OH, —CH(CH₃)CH₂OH, —CH₂CH₂CH₂OH, —OCH₃, —OCH₂CH₃, —OCH(CH₃)₂, and —OCH₂CH₂CH₃. In an even further aspect, each of R₂₆ and R_{26′} is independently selected from hydrogen, —OH, —CH₂OH, —CH₂CH₂OH, —OCH₃, and —OCH₂CH₃. In a still further aspect, each of R₂₆ and R_{26′} is independently selected from hydrogen, —OH, —CH₂OH, and —OCH₃.

In a further aspect, R₂₆ and R_{26′} together comprise oxo. In a still further aspect, each of R₂₆ and R_{26′} together comprise ═O.

In a further aspect, each of R₂₆ and R_{26′} is independently selected from hydrogen, —OH, and C1-C8 alkoxy. In a still further aspect, each of R₂₆ and R_{26′} is independently selected from hydrogen, —OH, and C1-C4 alkoxy. In yet a further aspect, each of R₂₆ and R_{26′} is independently selected from hydrogen, —OH, —OCH₃, —OCH₂CH₃, —OCH(CH₃)₂, and —OCH₂CH₂CH₃. In an even further aspect, each of R₂₆ and R_{26′} is independently selected from hydrogen, —OH, —OCH₃, and —OCH₂CH₃. In a still further aspect, each of R₂₆ and R_{26′} is independently selected from hydrogen, —OH, and —OCH₃.

In a further aspect, each of R₂₆ and R_{26′} is independently selected from hydrogen and —OH. In a still further aspect, each of R₂₆ and R_{26′} is —OH. In yet a further aspect, each of R₂₆ and R_{26′} is hydrogen.

m. R₂₇ Groups

In one aspect, R₂₇ is hydrogen or alkyl_{(C≤6)}. In one aspect, R₂₇ is selected from hydrogen and C1-C6 alkyl.

In a further aspect, R₂₇ is selected from hydrogen and C1-C6 alkyl. In a still further aspect, R₂₇ is selected from hydrogen and C1-C4 alkyl. In yet a further aspect, R₂₇ is selected from hydrogen, methyl, ethyl, n-propyl, and i-propyl. In an even further aspect, R₂₇ is selected from hydrogen, methyl, and ethyl. In a still further aspect, R₂₇ is selected from hydrogen and ethyl. In yet a further aspect, R₂₇ is selected from hydrogen and methyl.

In a further aspect, R₂₇ is C1-C6 alkyl. In a still further aspect, R₂₇ is C1-C4 alkyl. In yet a further aspect, R₂₇ is selected from methyl, ethyl, n-propyl, and i-propyl. In an even further aspect, R₂₇ is selected from methyl and ethyl. In a still further aspect, R₂₇ is ethyl. In yet a further aspect, R₂₇ is methyl.

In a further aspect, R₂₇ is hydrogen.

n. R₃₁ Groups

In one aspect, R₃₁, when present, is selected from hydrogen and C1-C4 alkyl. In a further aspect, R₃₁, when present, is hydrogen

In one aspect, R³¹, when present, is selected from hydrogen and C1-C12 alkyl. In a further aspect, R³¹, when present, is selected from hydrogen and C1-C8 alkyl.

In yet a further aspect, R₃₁, when present, is selected from hydrogen, methyl, ethyl, n-propyl, and i-propyl. In an even further aspect, R₃₁, when present, is selected from hydrogen, methyl, and ethyl. In a still further aspect, R₃₁, when present, is selected from hydrogen and ethyl. In yet a further aspect, R₃₁, when present, is selected from hydrogen and methyl.

In a further aspect, R₃₁, when present, is C1-C6 alkyl. In a still further aspect, R₃₁, when present, is C1-C4 alkyl. In yet a further aspect, R₃₁, when present, is selected from methyl, ethyl, n-propyl, and i-propyl. In an even further aspect, R₃₁, when present, is selected from methyl and ethyl. In a still further aspect, R₃₁, when present, is ethyl. In yet a further aspect, R₃₁, when present, is methyl.

o. R₄₁, R₄₂, R₄₄, R₄₅ₐ, and R_{45b} Groups

In one aspect, each occurrence of R₄₁, R₄₂, R₄₄, R₄₅ₐ, and R_{45b}, when present, is independently selected from hydrogen and C1-C12 alkyl. In a further aspect, each occurrence of R₄₁, R₄₂, R₄₄, R₄₅ₐ, and R_{45b}, when present, is independently selected from hydrogen and C1-C8 alkyl. In a still further aspect, each occurrence of R₄₁, R₄₂, R₄₄, R₄₅ₐ, and R_{45b}, when present, is independently selected from hydrogen and C1-C4 alkyl. In yet a further aspect, each occurrence of R₄₁, R₄₂, R₄₄, R₄₅ₐ, and R_{45b}, when present, is hydrogen.

In a further aspect, each occurrence of R₄₁, R₄₂, R₄₄, R₄₅ₐ, and R_{45b}, when present, is independently selected from hydrogen, methyl, ethyl, n-propyl, i-propyl, n-butyl, s-butyl, i-butyl, and t-butyl. In a still further aspect, each occurrence of R₄₁, R₄₂, R₄₄, R₄₅ₐ, and R_{45b}, when present, is independently selected from hydrogen, methyl, ethyl, n-propyl, and i-propyl. In yet a further aspect, each occurrence of R₄₁, R₄₂, R₄₄, R₄₅ₐ, and R_{45b}, when present, is independently selected from hydrogen, methyl, and ethyl. In an even further aspect, each occurrence of R₄₁, R₄₂, R₄₄, R₄₅ₐ, and R_{45b}, when present, is independently selected from hydrogen and ethyl. In a still further aspect, each occurrence of R₄₁, R₄₂, R₄₄, R₄₅ₐ, and R_{45b}, when present, is independently selected from hydrogen and methyl.

In a further aspect, each occurrence of R₄₁, R₄₂, R₄₄, R₄₅ₐ, and R_{45b}, when present, is independently selected from C1-C12 alkyl. In a still further aspect, each occurrence of R₄₁, R₄₂, R₄₄, R₄₅ₐ, and R_{45b}, when present, is independently selected from C1-C8 alkyl. In yet a further aspect, each occurrence of R₄₁, R₄₂, R₄₄, R₄₅ₐ, and R_{45b}, when present, is independently selected from C1-C4 alkyl. In an even further aspect, each occurrence of R₄₁, R₄₂, R₄₄, R₄₅ₐ, and R_{45b}, when present, is independently selected from methyl, ethyl, n-propyl, and i-propyl. In a still further aspect, each occurrence of R₄₁, R₄₂, R₄₄, R₄₅ₐ, and R_{45b}, when present, is independently selected from methyl and ethyl. In yet a further aspect, each occurrence of R₄₁, R₄₂, R₄₄, R₄₅ₐ, and R_{45b}, when present, is ethyl. In an even further aspect, each occurrence of R₄₁, R₄₂, R₄₄, R₄₅ₐ, and R_{45b}, when present, is methyl.

p. R₄₃ Groups

In one aspect, each occurrence of R₄₃, when present, is independently selected from hydrogen, C1-C12 alkyl, and monocyclic aryl monosubstituted with a methyl group. In a further aspect, each occurrence of R₄₃, when present, is independently selected from hydrogen, C1-C8 alkyl, and monocyclic aryl monosubstituted with a methyl group. In a still further aspect, each occurrence of R₄₃, when present, is independently selected from hydrogen, C1-C4 alkyl, and monocyclic aryl monosubstituted with a methyl group. In yet a further aspect, each occurrence of R₄₃, when present, is independently selected from hydrogen, methyl, ethyl, n-propyl, i-propyl, and monocyclic aryl monosubstituted with a methyl group. In an even further aspect, each occurrence of R₄₃, when present, is independently selected from hydrogen, methyl, ethyl, and monocyclic aryl monosubstituted with a methyl group. In a still further aspect, each occurrence of R₄₃, when present, is independently selected from hydrogen, methyl, and monocyclic aryl monosubstituted with a methyl group.

In a further aspect, each occurrence of R₄₃, when present, is hydrogen.

In a further aspect, each occurrence of R₄₃, when present, is independently C1-C12 alkyl. In a still further aspect, each occurrence of R₄₃, when present, is independently C1-C8 alkyl. In yet a further aspect, each occurrence of R₄₃, when present, is independently C1-C4 alkyl. In an even further aspect, each occurrence of R₄₃, when present, is independently selected from methyl, ethyl, n-propyl, and i-propyl. In a still further aspect, each occurrence of R₄₃, when present, is independently selected from methyl and ethyl. In yet a further aspect, each occurrence of R₄₃, when present, is ethyl. In an even further aspect, each occurrence of R₄₃, when present, is methyl.

In a further aspect, each occurrence of R₄₃, when present, is monocyclic aryl monosubstituted with a methyl group. In a still further aspect, each occurrence of R₄₃, when present, is a structure represented by a formula:

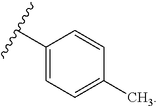 [SMILES: *c1ccc(C)cc1]

q. R₄₆ Groups

In one aspect, each occurrence of R₄₆, when present, is independently selected from hydrogen and C1-C12 alkyl. In a further aspect, each occurrence of R₄₆, when present, is independently selected from hydrogen and C1-C8 alkyl. In a still further aspect, each occurrence of R₄₆, when present, is independently selected from hydrogen and C1-C4 alkyl. In yet a further aspect, each occurrence of R₄₆, when present, is independently selected from hydrogen, methyl, ethyl, n-propyl, and i-propyl. In an even further aspect, each occurrence of R₄₆, when present, is independently selected from hydrogen, methyl, and ethyl. In a still further aspect, each occurrence of R₄₆, when present, is independently selected from hydrogen and ethyl. In yet a further aspect, each occurrence of R₄₆, when present, is independently selected from hydrogen and methyl.

In a further aspect, each occurrence of R₄₆, when present, is hydrogen.

In a further aspect, each occurrence of R₄₆, when present, is C1-C12 alkyl. In a still further aspect, each occurrence of R₄₆, when present, is C1-C8 alkyl. In yet a further aspect, each occurrence of R₄₆, when present, is C1-C4 alkyl. In an even further aspect, each occurrence of R₄₆, when present, is independently selected from methyl, ethyl, n-propyl, and i-propyl. In a still further aspect, each occurrence of R₄₆, when present, is independently selected from methyl and ethyl. In yet a further aspect, each occurrence of R₄₆, when present, is ethyl. In an even further aspect, each occurrence of R₄₆, when present, is methyl.

r. R₅₁ and R₅₂ Groups

In one aspect, each of R₅₁ and R₅₂ is independently halogen or each of R₅₁ and R₅₂ together comprise —O— or —N(R₅₃)—.

In a further aspect, each of R₅₁ and R₅₂ is independently halogen. In a still further aspect, each of R₅₁ and R₅₂ is independently selected from —F and —Cl. In yet a further aspect, each of R₅₁ and R₅₂ is —Cl. In an even further aspect, each of R₅₁ and R₅₂ is —F.

In a further aspect, each of R₅₁ and R₅₂ together comprise —O— or —N(R₅₃)—. In a still further aspect, each of R₅₁ and R₅₂ together comprise —O—. In yet a further aspect, each of R₅₁ and R₅₂ together comprise —N(R₅₃)—.

s. R₅₃ Groups

In one aspect, R₅₃, when present, is selected from hydrogen, C1-C4 alkyl, —SO₂R₅₄, and a structure having a formula:

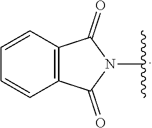 [SMILES: *N1C(=O)c2ccccc2C1=O]

In a further aspect, R₅₃, when present, is selected from hydrogen and C1-C4 alkyl. In a still further aspect, R₅₃, when present, is selected from hydrogen, methyl, ethyl, n-propyl, and i-propyl. In yet a further aspect, R₅₃, when present, is selected from hydrogen, methyl, and ethyl. In an even further aspect, R₅₃, when present, is selected from hydrogen and ethyl. In a still further aspect, R₅₃, when present, is selected from hydrogen and methyl.

In a further aspect, R₅₃, when present, is hydrogen.

In a further aspect, R₅₃, when present, is C1-C4 alkyl. In a still further aspect, R₅₃, when present, is selected from methyl, ethyl, n-propyl, and i-propyl. In yet a further aspect, R₅₃, when present, is selected from methyl and ethyl. In an even further aspect, R₅₃, when present, is ethyl. In a still further aspect, R₅₃, when present, is methyl.

In a further aspect, R₅₃, when present, is selected from —SO₂R₅₄ and a structure having a formula:

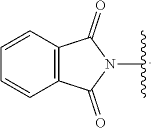 [SMILES: *N1C(=O)c2ccccc2C1=O]

In a further aspect, R₅₃, when present, is —SO₂R₅₄.

In a further aspect, R₅₃, when present, is a structure having a formula:

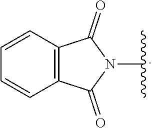 [SMILES: *N1C(=O)c2ccccc2C1=O]

t. R₅₄ Groups

In one aspect, R₅₄, when present, is selected from hydrogen, C1-C4 alkyl, —CH₂CH₂Si(CH₃)₃, and monocyclic aryl monosubstituted with a methyl group. In a further aspect, R₅₄, when present, is hydrogen.

In a further aspect, R₅₄, when present, is selected from hydrogen, methyl, ethyl, n-propyl, i-propyl, —CH₂CH₂Si(CH₃)₃, and monocyclic aryl monosubstituted with a methyl group. In a still further aspect, R₅₄, when present, is selected from hydrogen, methyl, ethyl, —CH₂CH₂Si(CH₃)₃, and monocyclic aryl monosubstituted with a methyl group. In yet a further aspect, R₅₄, when present, is selected from hydrogen, methyl, —CH₂CH₂Si(CH₃)₃, and monocyclic aryl monosubstituted with a methyl group.

In a further aspect, R₅₄, when present, is selected from hydrogen, methyl, ethyl, n-propyl, i-propyl, n-butyl, i-butyl, s-butyl, and t-butyl. In a still further aspect, R₅₄, when present, is selected from hydrogen, methyl, ethyl, n-propyl, and i-propyl. In yet a further aspect, R₅₄, when present, is selected from hydrogen, methyl, and ethyl. In an even further aspect, R₅₄, when present, is selected from hydrogen and ethyl. In a still further aspect, R₅₄, when present, is selected from hydrogen and methyl.

In a further aspect, R₅₄, when present, is selected from methyl, ethyl, n-propyl, i-propyl, n-butyl, i-butyl, s-butyl, and t-butyl. In a still further aspect, R₅₄, when present, is selected from methyl, ethyl, n-propyl, and i-propyl. In yet a further aspect, R₅₄, when present, is selected from methyl and ethyl. In an even further aspect, R₅₄, when present, is ethyl. In a still further aspect, R₅₄, when present, is methyl.

In a further aspect, R₅₄, when present, is selected from —CH₂CH₂Si(CH₃)₃ and monocyclic aryl monosubstituted with a methyl group. In a still further aspect, R₅₄, when present, is —CH₂CH₂Si(CH₃)₃. In yet a further aspect, R₅₄, when present, is monocyclic aryl monosubstituted with a methyl group. In an even further aspect, R₅₄, when present, is a structure represented by a formula:

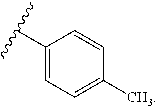 [SMILES: *c1ccc(C)cc1]

u. R^{x} Groups

In one aspect, each Rx is independently hydrogen or alkyl_{(C≤6)}. In one aspect, R^{x}, when present, is selected from hydrogen and C1-C6 alkyl.

In a further aspect, R^{x} is selected from hydrogen and C1-C6 alkyl. In a still further aspect, R^{x} is selected from hydrogen and C1-C4 alkyl. In yet a further aspect, R^{x} is selected from hydrogen, methyl, ethyl, n-propyl, and i-propyl. In an even further aspect, R^{x} is selected from hydrogen, methyl, and ethyl. In a still further aspect, R^{x} is selected from hydrogen and ethyl. In yet a further aspect, R^{x} is selected from hydrogen and methyl.

In a further aspect, R^{x} is C1-C6 alkyl. In a still further aspect, R^{x} is C1-C4 alkyl. In yet a further aspect, R^{x} is selected from methyl, ethyl, n-propyl, and i-propyl. In an even further aspect, R^{x} is selected from methyl and ethyl. In a still further aspect, R^{x} is ethyl. In yet a further aspect, R^{x} is methyl.

In a further aspect, R^{x} is hydrogen.

v. Cy₁ Groups

In one aspect, each occurrence of Cy₁, when present, is independently heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, each occurrence of Cy₁, when present, is independently heterocycloalkyl substituted with 0, 1, or 2 groups selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy₁, when present, is independently heterocycloalkyl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Cy₁, when present, is independently heterocycloalkyl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Cy₁, when present, is independently unsubstituted heterocycloalkyl.

In a further aspect, each occurrence of Cy₁, when present, is independently heterocycloalkyl containing at least one N and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy₁, when present, is independently heterocycloalkyl containing at least one N and substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Cy₁, when present, is independently heterocycloalkyl containing at least one N and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Cy₁, when present, is independently heterocycloalkyl containing at least one N and monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy₁, when present, is independently heterocycloalkyl containing at least one N and unsubstituted.

In a further aspect, each occurrence of Cy₁, when present, is independently selected from aziridinyl, oxiranyl, piperidinyl, pyrrolidinyl, tetrahydro-2H-pyranyl, tetrahydro-2H-thiopyranyl, tetrahdryofuranyl, tetrahydrothiophenyl, and thiiranyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy₁, when present, is independently selected from aziridinyl, oxiranyl, piperidinyl, pyrrolidinyl, tetrahydro-2H-pyranyl, tetrahydro-2H-thiopyranyl, tetrahdryofuranyl, tetrahydrothiophenyl, and thiiranyl and substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Cy₁, when present, is independently selected from aziridinyl, oxiranyl, piperidinyl, pyrrolidinyl, tetrahydro-2H-pyranyl, tetrahydro-2H-thiopyranyl, tetrahdryofuranyl, tetrahydrothiophenyl, and thiiranyl and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Cy₁, when present, is independently selected from aziridinyl, oxiranyl, piperidinyl, pyrrolidinyl, tetrahydro-2H-pyranyl, tetrahydro-2H-thiopyranyl, tetrahdryofuranyl, tetrahydrothiophenyl, and thiiranyl and monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Cy₁, when present, is independently selected from aziridinyl, oxiranyl, piperidinyl, pyrrolidinyl, tetrahydro-2H-pyranyl, tetrahydro-2H-thiopyranyl, tetrahdryofuranyl, tetrahydrothiophenyl, and thiiranyl and unsubstituted.

w. Ar₁ Groups

In one aspect, Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, Ar₁, when present, is selected from monocyclic 6-membered aryl and anthracene-9,10-dionyl, and is substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar₁, when present, is selected from monocyclic 6-membered aryl and anthracene-9,10-dionyl, and is substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar₁, when present, is selected from monocyclic 6-membered aryl and anthracene-9,10-dionyl, and is monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar₁, when present, is selected from monocyclic 6-membered aryl and anthracene-9,10-dionyl, and is unsubstituted.

In a further aspect, Ar₁, when present, is monocyclic 6-membered aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar₁, when present, is monocyclic 6-membered aryl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar₁, when present, is monocyclic 6-membered aryl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar₁, when present, is monocyclic 6-membered aryl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar₁, when present, is unsubstituted monocyclic 6-membered aryl.

In a further aspect, Ar₁, when present, is anthracene-9,10-dionyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar₁, when present, is anthracene-9,10-dionyl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, Ar₁, when present, is anthracene-9,10-dionyl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, Ar₁, when present, is anthracene-9,10-dionyl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, Ar₁, when present, is unsubstituted anthracene-9,10-dionyl.

x. Ar₂ Groups

In one aspect, Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

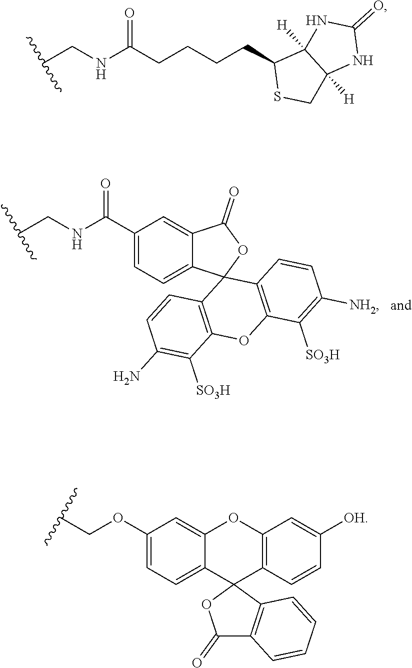 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; "and" ; *COc1ccc2c(c1)Oc1cc(O)ccc1C21OC(=O)c2ccccc21]

In a further aspect, Ar₂, when present, is selected from monocyclic 6-membered aryl, triazolyl, and anthracene-9,10-dionyl, and is substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

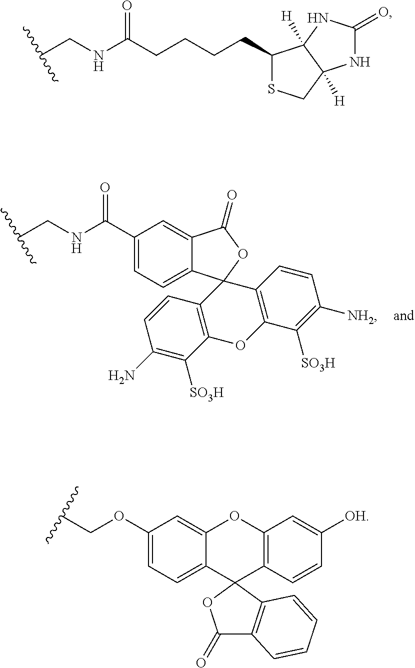 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is selected from monocyclic 6-membered aryl, triazolyl, and anthracene-9,10-dionyl, and is substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

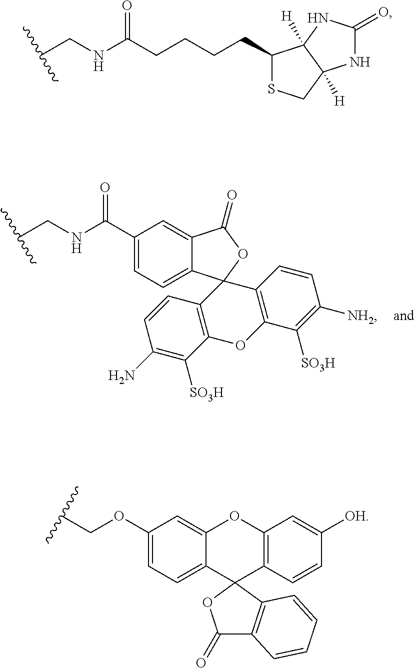 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In yet a further aspect, Ar₂, when present, is selected from monocyclic 6-membered aryl, triazolyl, and anthracene-9,10-dionyl, and is monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

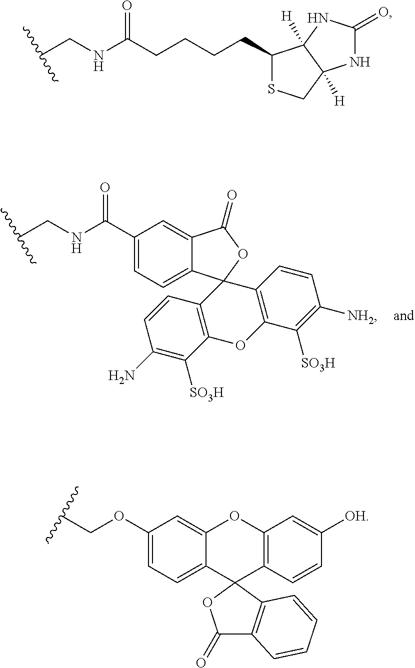 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In an even further aspect, Ar₂, when present, is selected from monocyclic 6-membered aryl, triazolyl, and anthracene-9,10-dionyl, and is unsubstituted.

In a further aspect, Ar₂, when present, is monocyclic 6-membered aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

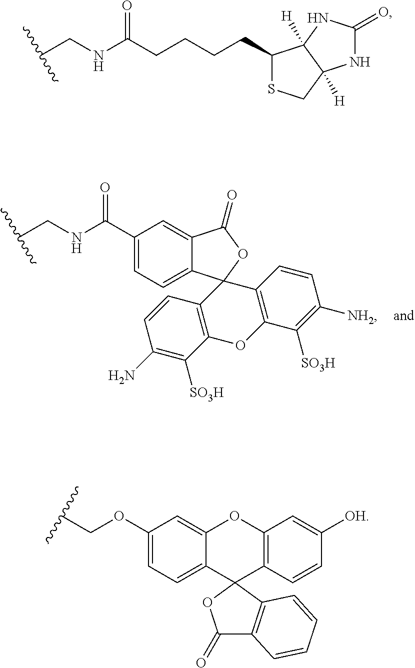 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is monocyclic 6-membered aryl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

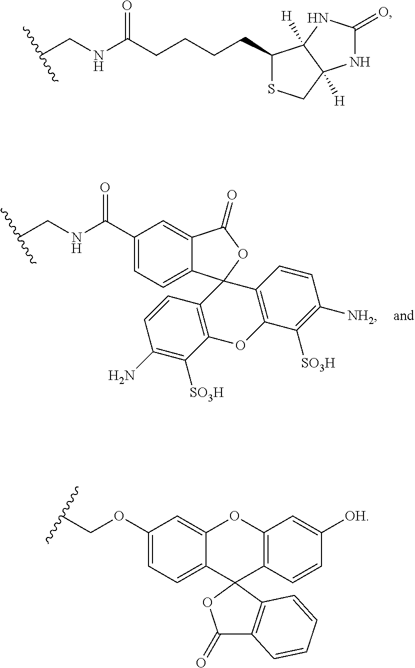 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In yet a further aspect, Ar₂, when present, is monocyclic 6-membered aryl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

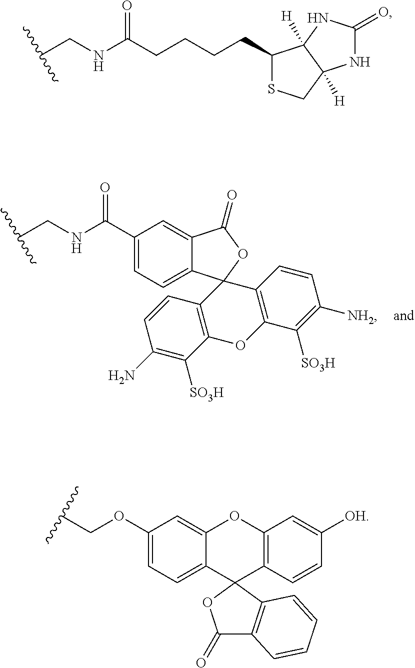 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In an even further aspect, Ar₂, when present, is monocyclic 6-membered aryl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

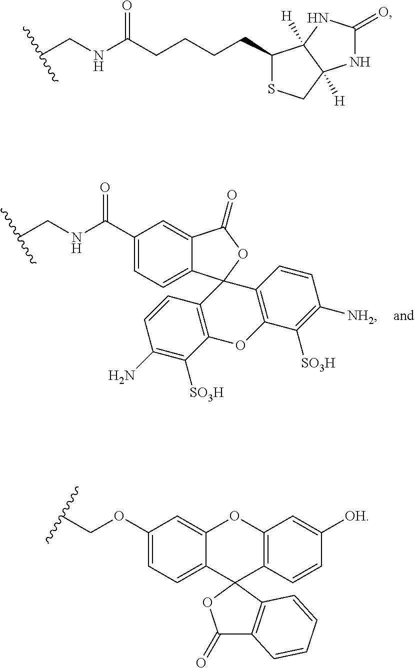 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is unsubstituted monocyclic 6-membered aryl.

In a further aspect, Ar₂, when present, is triazolyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

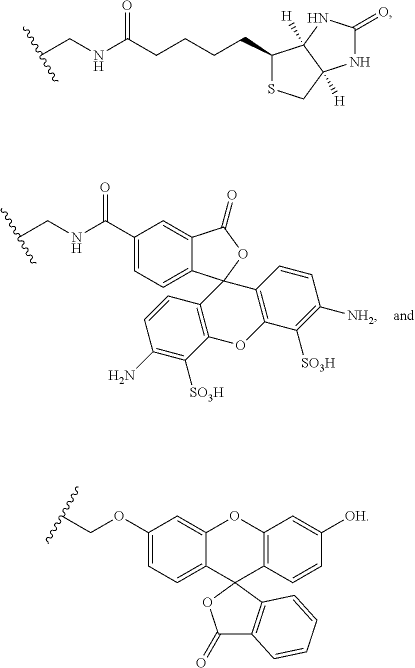 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is triazolyl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

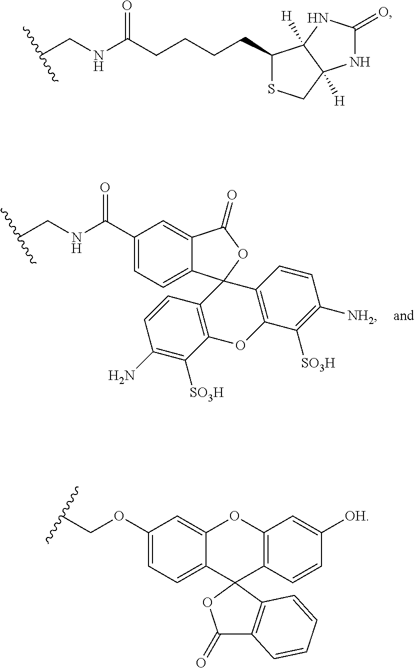 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In yet a further aspect, Ar₂, when present, is triazolyl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

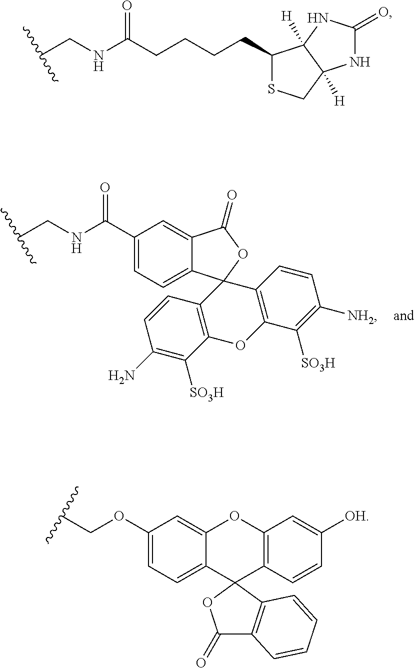 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In an even further aspect, Ar₂, when present, is triazolyl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

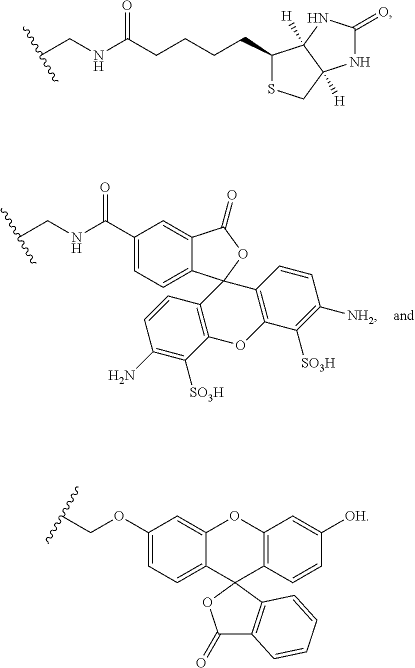 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is unsubstituted triazolyl.

In a further aspect, Ar₂, when present, is anthracene-9,10-dionyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

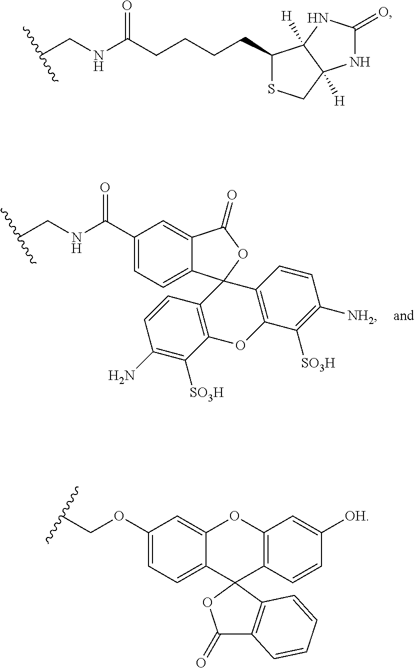 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is anthracene-9,10-dionyl substituted with 0, 1, or 2 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

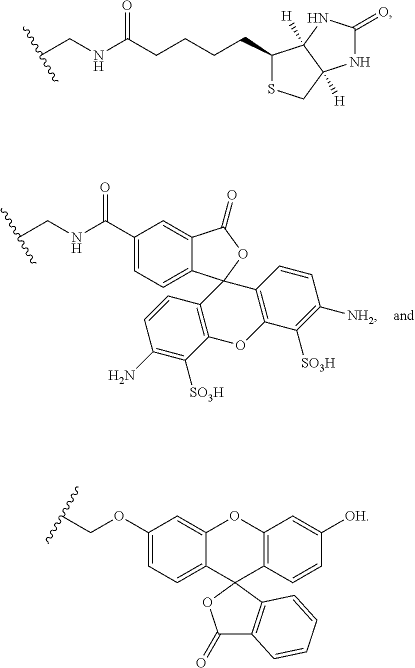 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In yet a further aspect, Ar₂, when present, is anthracene-9,10-dionyl substituted with 0 or 1 group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

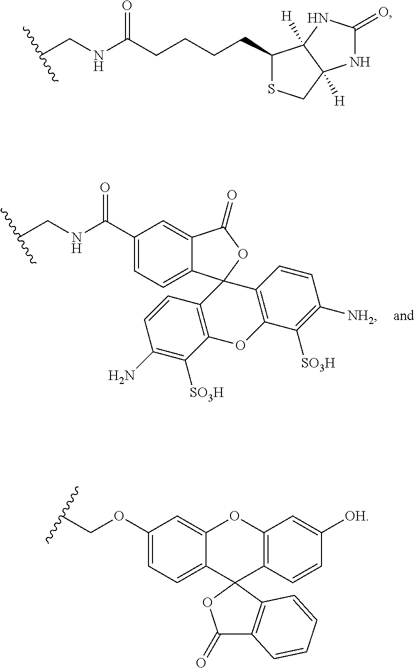 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In an even further aspect, Ar₂, when present, is anthracene-9,10-dionyl monosubstituted with a group selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from:

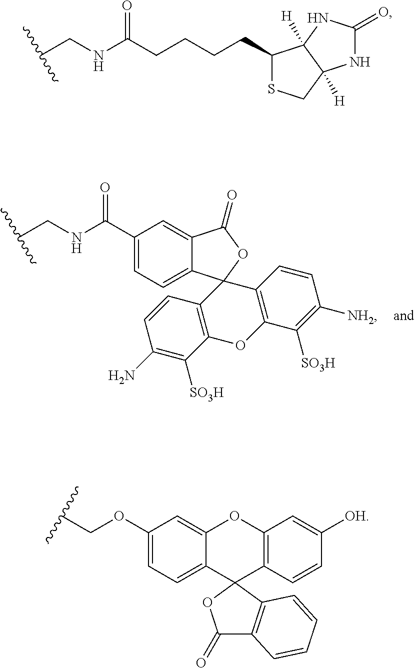 [SMILES: *CNC(=O)CCCC[C@@H]1SC[C@@H]2NC(=O)N[C@@H]21 ; *CNC(=O)c1ccc2c(c1)C(=O)OC21c2ccc(N)c(S(=O)(=O)O)c2Oc2c1ccc(N)c2S(=O)(=O)O ; *COc1ccc2c(c1)Oc1cc(O)ccc1C21OC(=O)c2ccccc21]

In a still further aspect, Ar₂, when present, is unsubstituted anthracene-9,10-dionyl.

y. Ar₃ Groups

In one aspect, each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a further aspect, each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and unsubstituted.

In a further aspect, each occurrence of Ar₃, when present, is independently monocyclic aryl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar₃, when present, is independently monocyclic aryl substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar₃, when present, is independently monocyclic aryl substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect, each occurrence of Ar₃, when present, is independently monocyclic aryl monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar₃, when present, is independently unsubstituted monocyclic aryl.

In a further aspect, each occurrence of Ar₃, when present, is independently selected from morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar₃, when present, is independently selected from morpholinyl, anilinyl, indolyl, pyrrolyl, imidazolyl, benzimidazolyl, pyrazolyl, guanidinyl, and piperazinyl and substituted with 0, 1, or 2 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In yet a further aspect, each occurrence of Ar₃, when present, is independently selected from morpholinyl, anilinyl, indolyl, pyrrolyl, imidazolyl, benzimidazolyl, pyrazolyl, guanidinyl, and piperazinyl and substituted with 0 or 1 group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In an even further aspect each occurrence of Ar₃, when present, is independently selected from morpholinyl, anilinyl, indolyl, pyrrolyl, imidazolyl, benzimidazolyl, pyrazolyl, guanidinyl, and piperazinyl and monosubstituted with a group selected from halogen, —OH, —NH₂, C1-C4 alkoxy, C1-C4 hydroxy, C1-C4 aminoalkyl, C1-C4 alkylamino, and (C1-C4)(C1-C4) dialkylamino. In a still further aspect, each occurrence of Ar₃, when present, is independently selected from morpholinyl, anilinyl, indolyl, pyrrolyl, imidazolyl, benzimidazolyl, pyrazolyl, guanidinyl, and piperazinyl and unsubstituted.

### 2. Example Compounds

In one aspect, a compound can be present as one or more of the following structures:

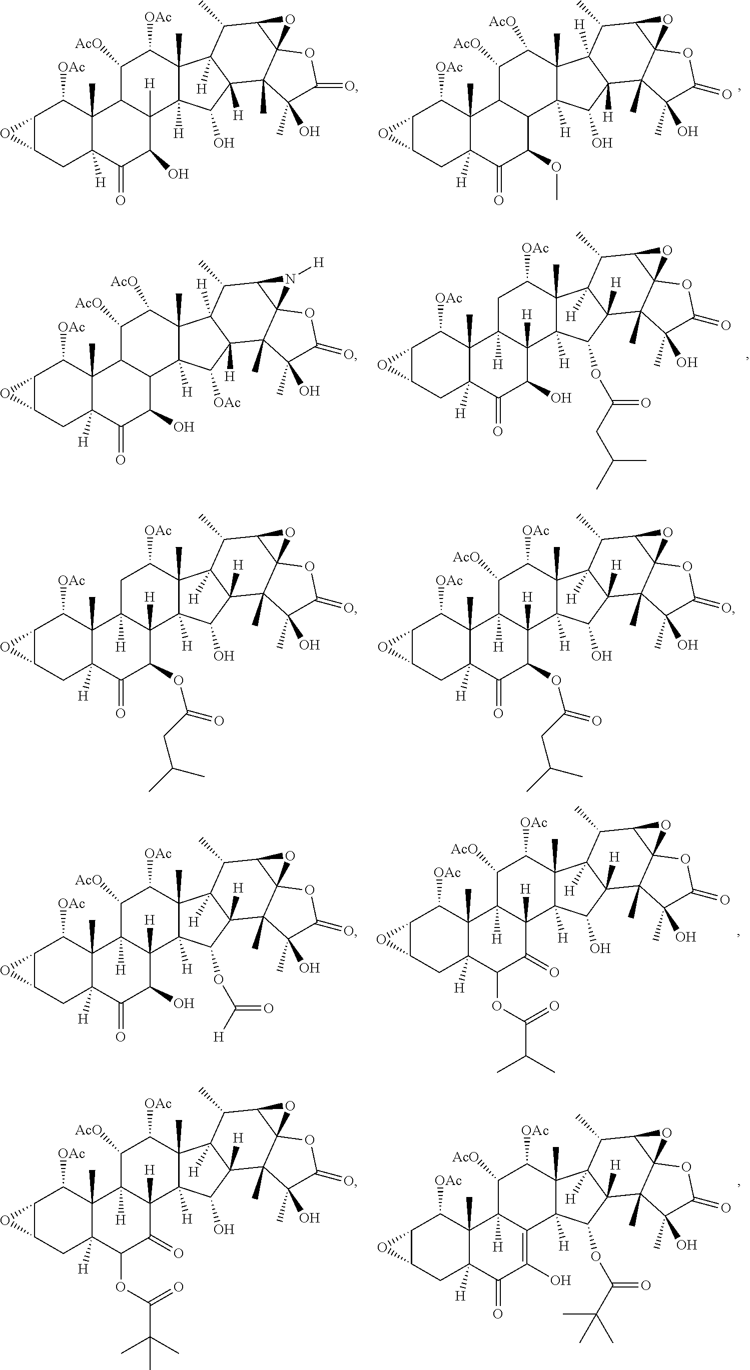

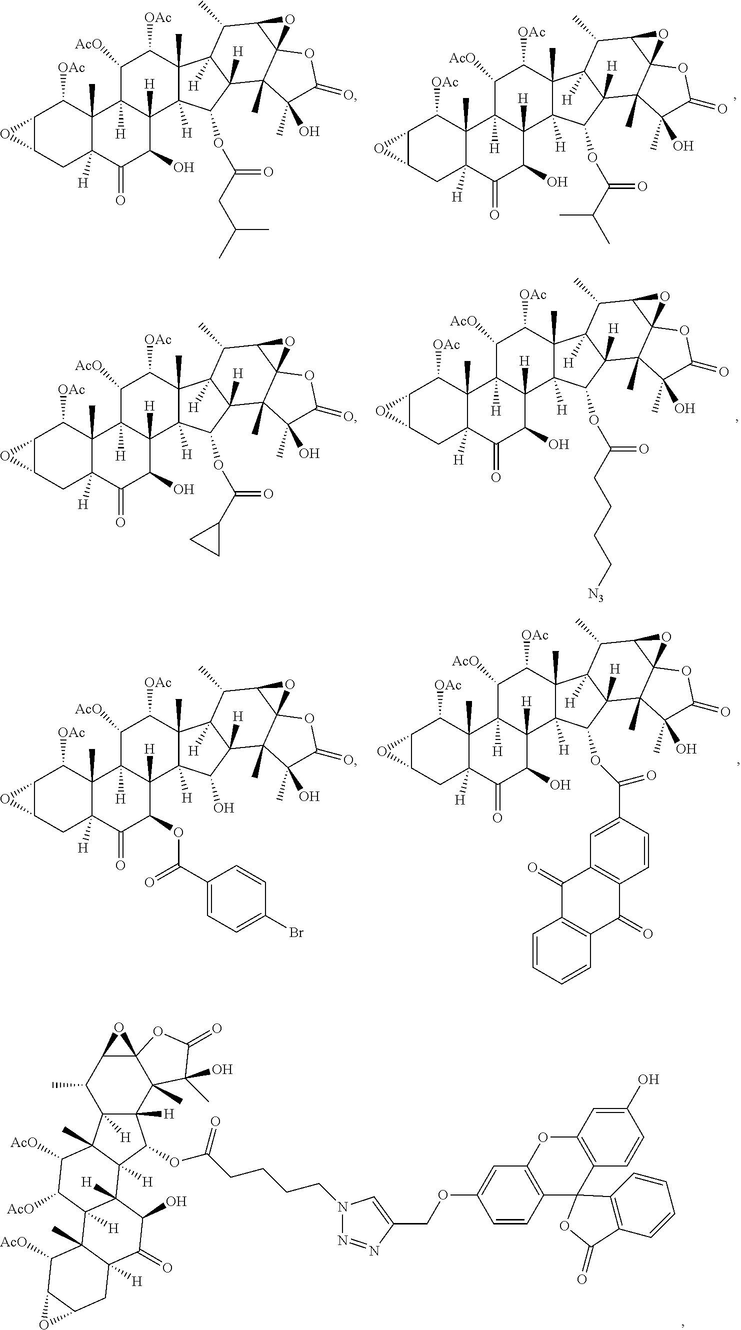

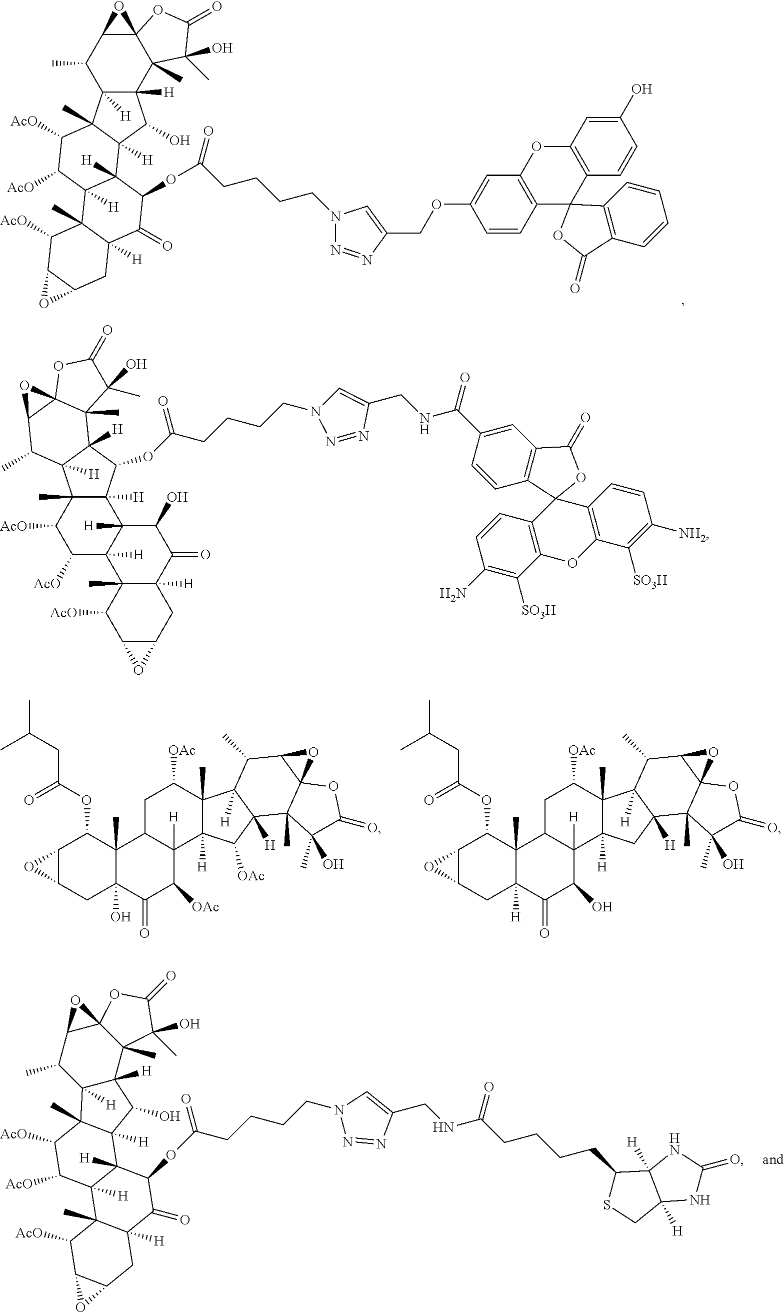

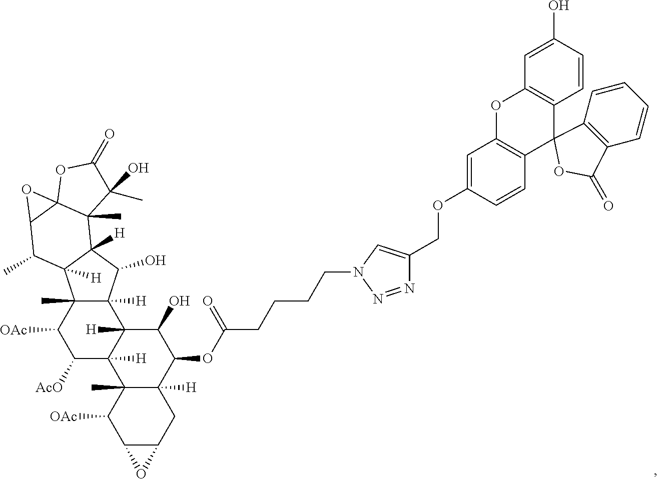

or a pharmaceutically acceptable salt thereof.

### 3. Prophetic Compound Examples

The following compound examples are prophetic, and can be prepared using the synthesis methods described herein above and other general methods as needed as would be known to one skilled in the art. It is anticipated that the prophetic compounds would be active as microtubule stabilizers, and such activity can be determined using the assay methods described herein.

In one aspect, a compound can be selected from:

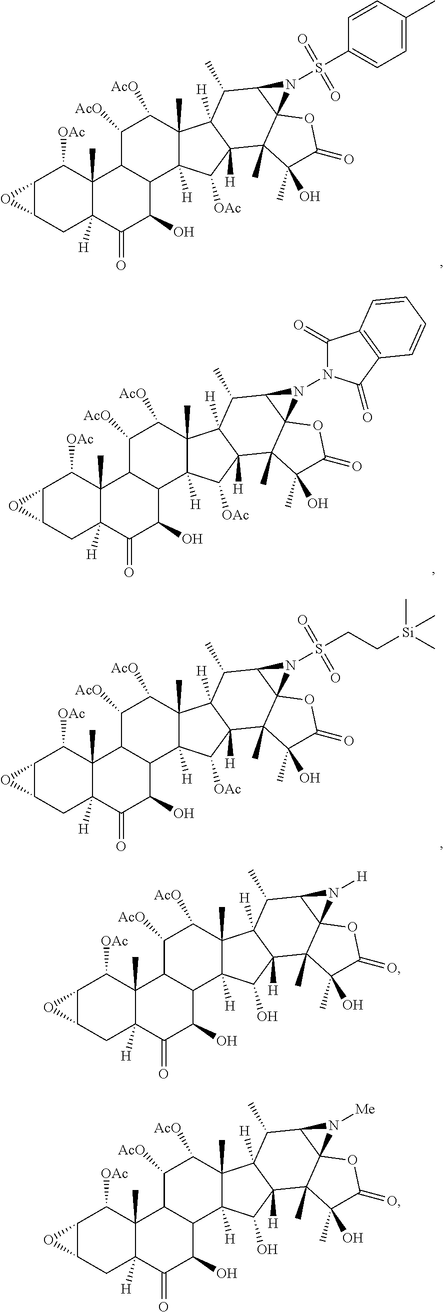

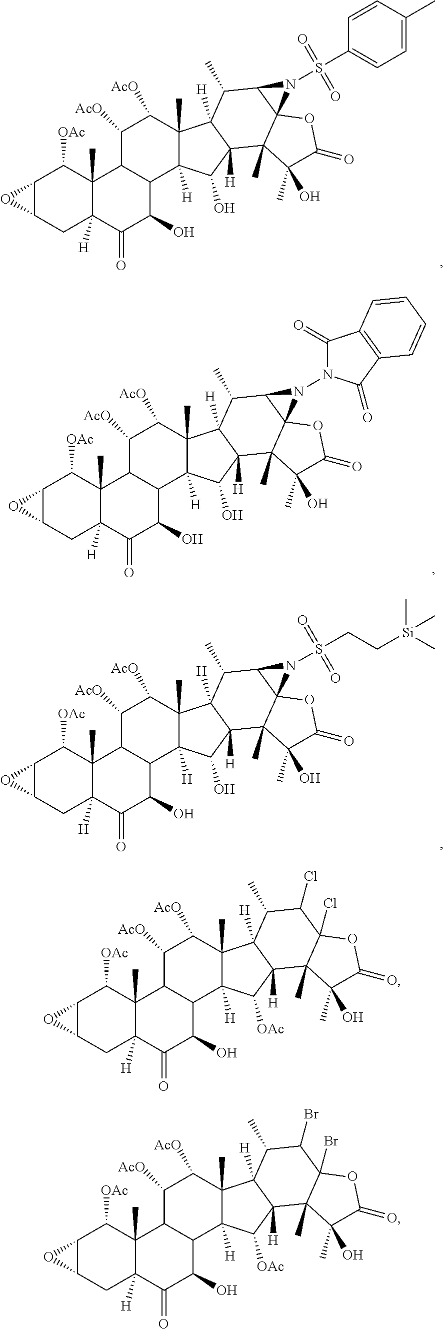

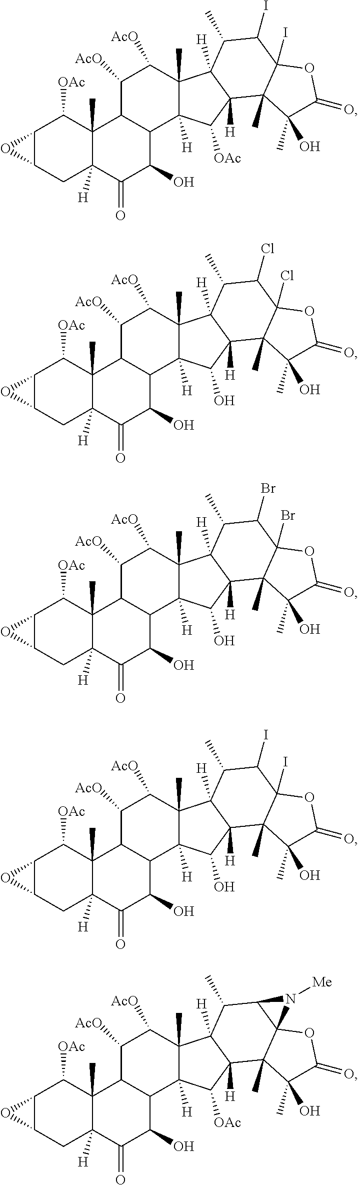

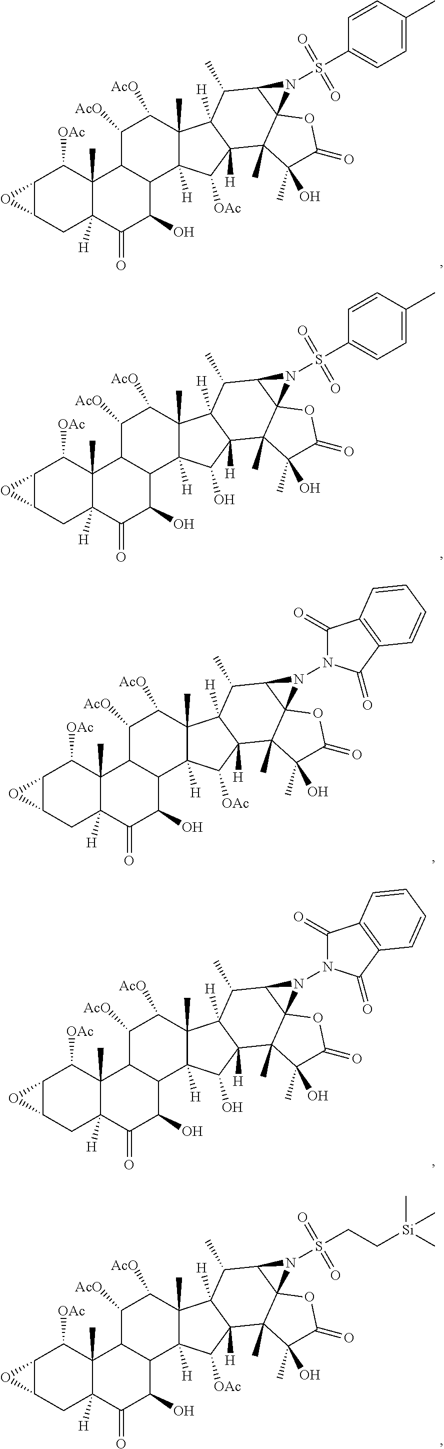

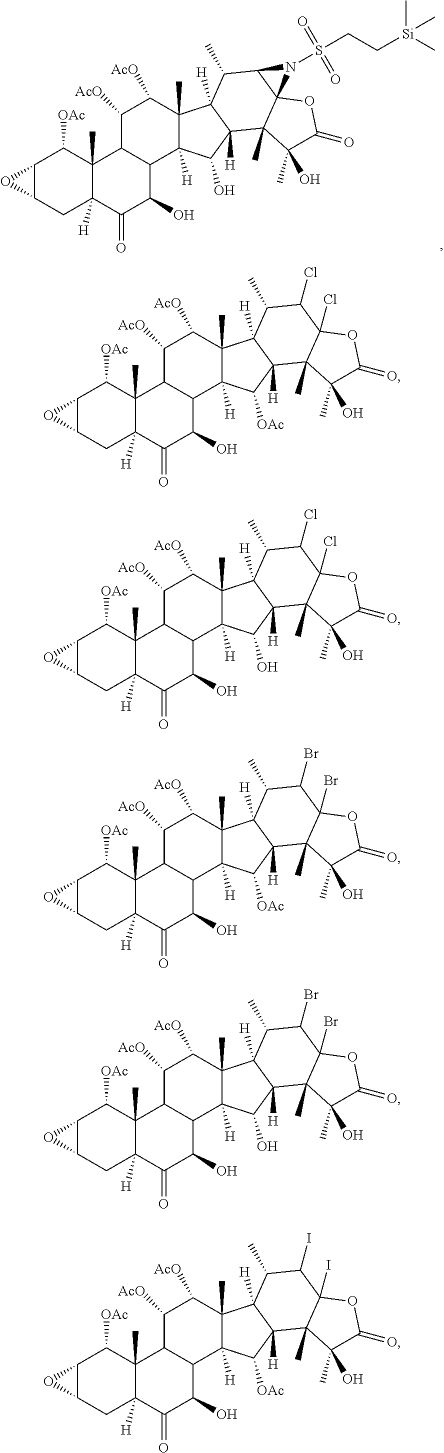

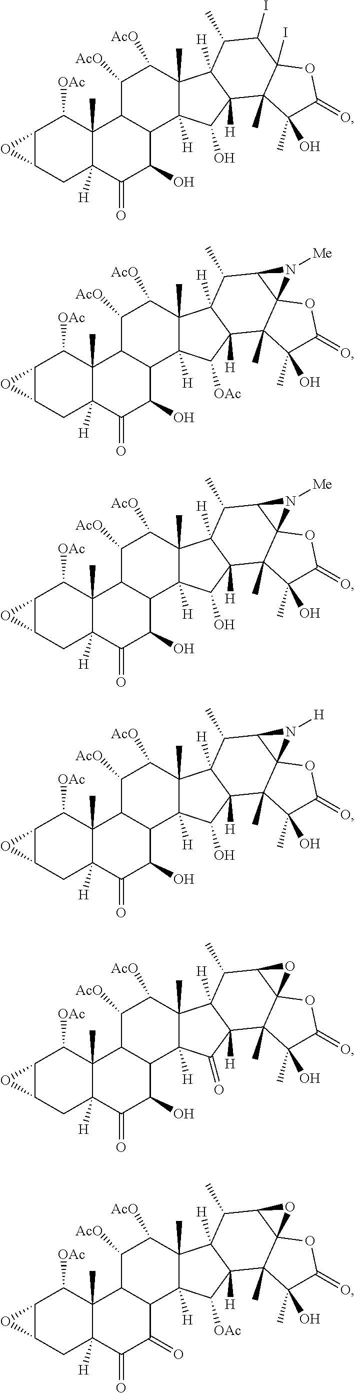

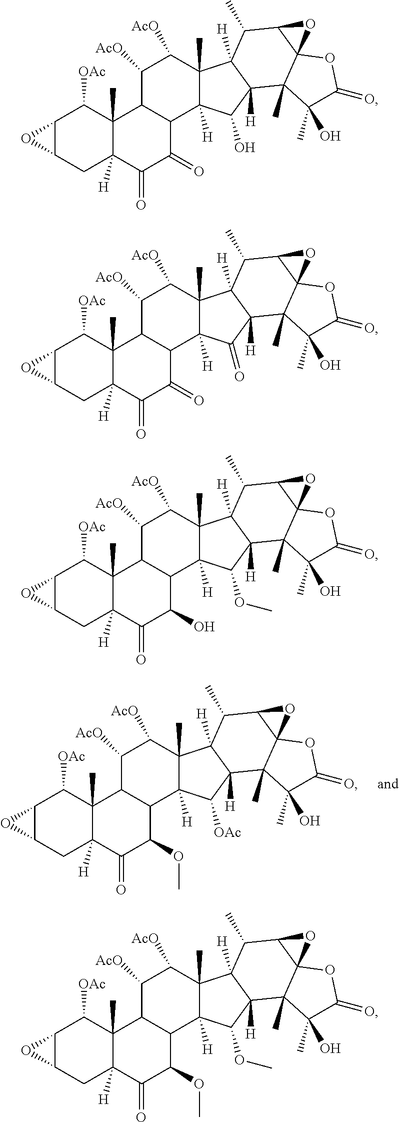

or a pharmaceutically acceptable derivative thereof.

In one aspect, a compound can be selected from:

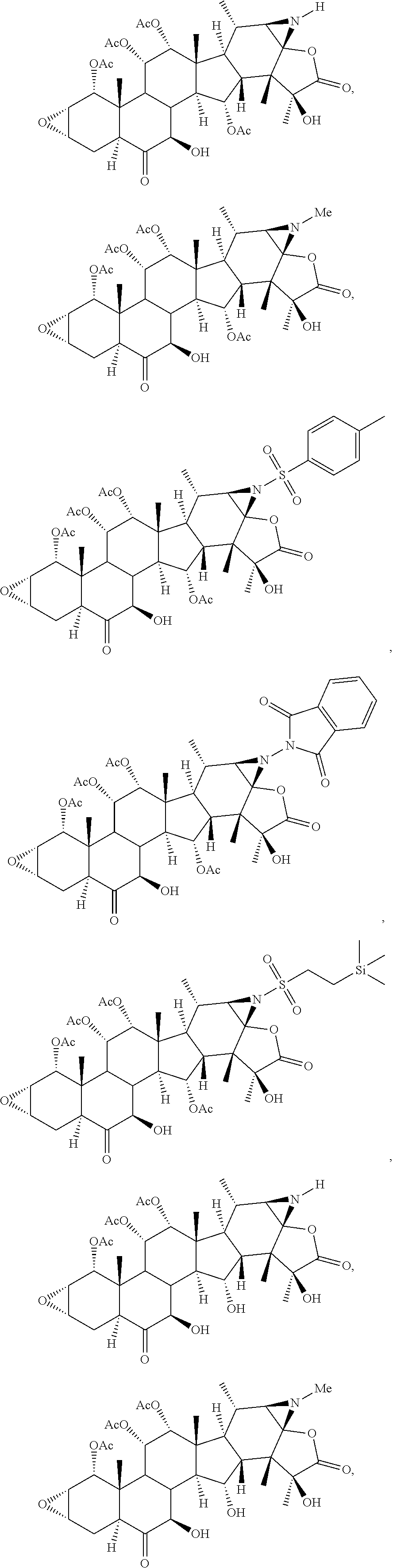

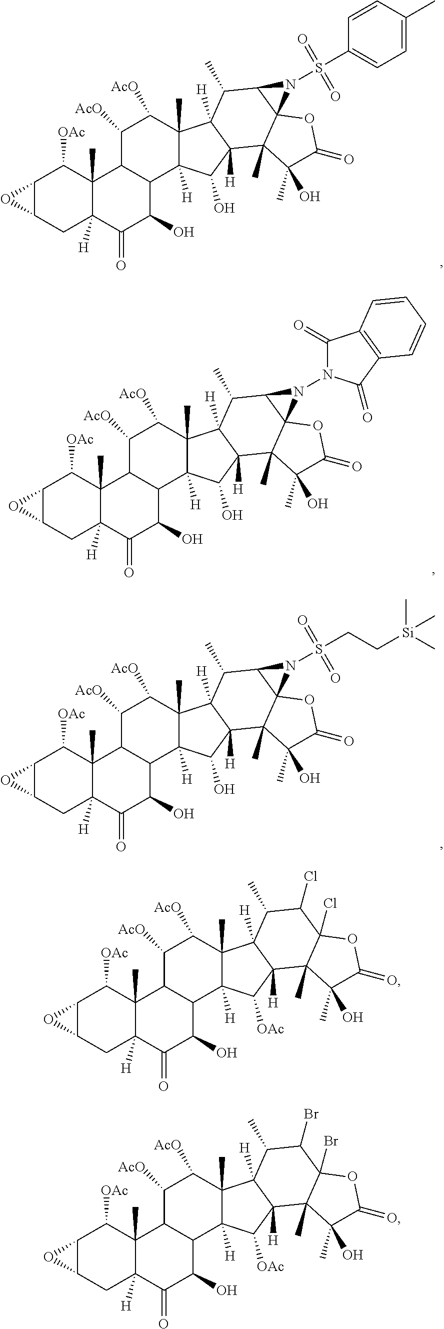

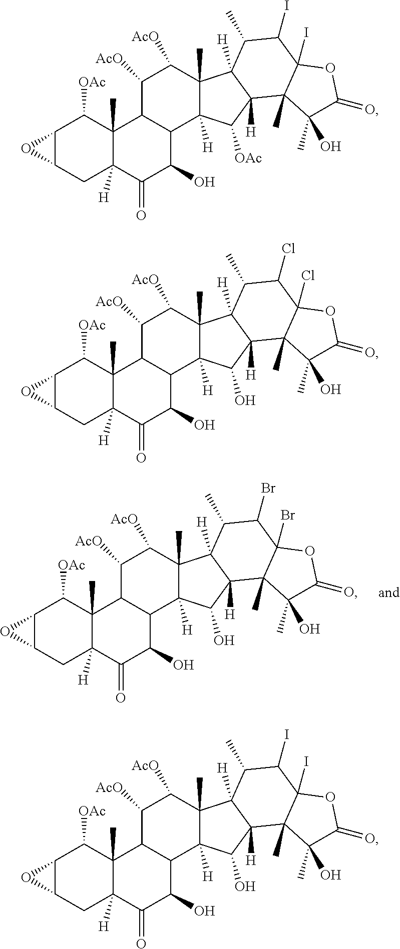

or a pharmaceutically acceptable derivative thereof.

In one aspect, a compound can be selected from:

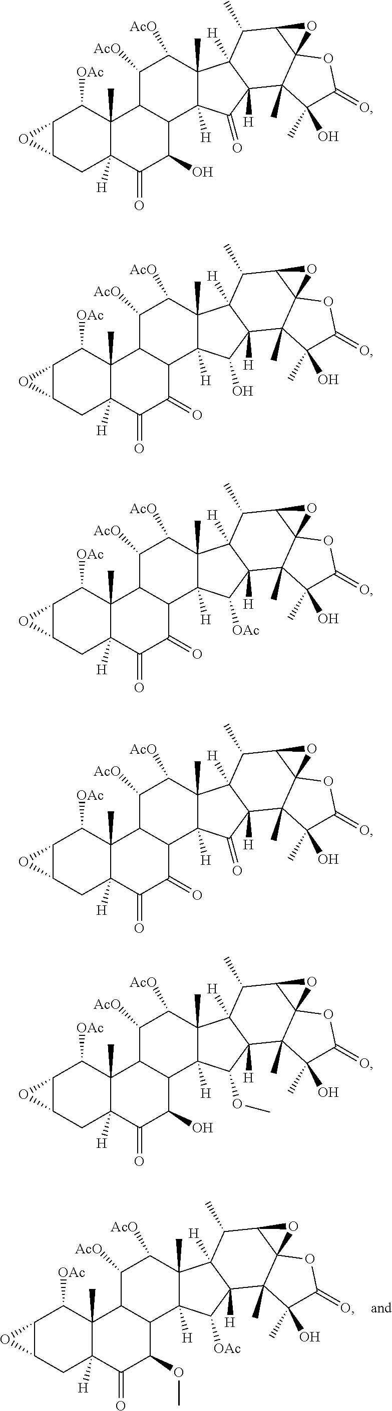

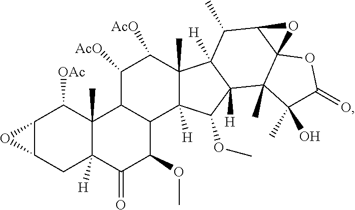 [SMILES: CO[C@H]1[C@@H]2[C@H]([C@H](C)[C@H]3O[C@]34OC(=O)[C@@](C)(O)[C@]24C)[C@]2(C)[C@@H]1C1C([C@H](OC(C)=O)[C@@H]2OC(C)=O)[C@]2(C)[C@H](C[C@@H]3O[C@@H]3[C@@H]2OC(C)=O)C(=O)[C@@H]1OC]

or a pharmaceutically acceptable derivative thereof.

### C. METHODS OF MAKING A COMPOUND

Methods for isolating and generating taccalonolide compounds by semi-synthesis according to the present invention are provided by the examples. Those of skill in the art would recognize similar methodologies that may also be employed.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VI, as described and exemplified below. The following examples are provided so that the invention might be more fully understood, are illustrative only, and should not be construed as limiting.

1. Route I

In one aspect, substituted small molecule modulators of microtubule function can be prepared as shown below.

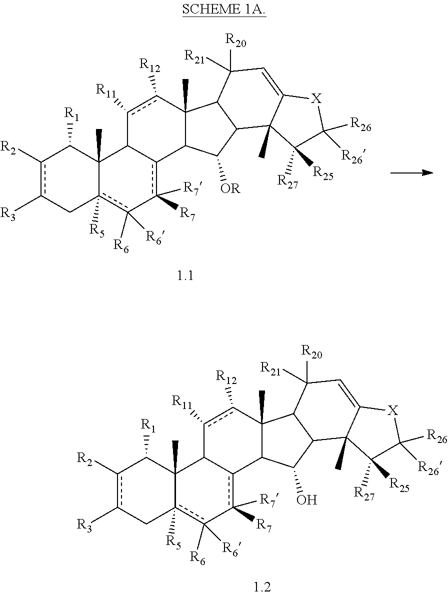

Compounds are represented in generic form, with substituents as noted in compound descriptions elsewhere herein and wherein R is —C(O)(C1-30). A more specific example is set forth below.

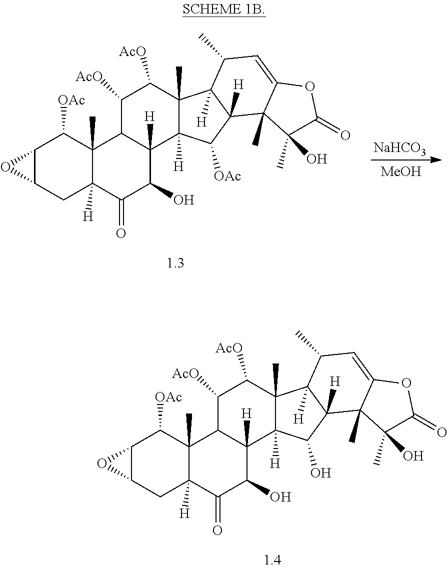

In one aspect, compounds of type 1.4, and similar compounds, can be prepared according to reaction Scheme 1B above. Thus, compounds of type 1.2 can be prepared by a hydrolysis reaction of an appropriate acyl analog, e.g., 1.1 as shown above. Appropriate acyl analogs are commercially available or prepared or isolated by methods known to one skilled in the art. The hydrolysis reaction is carried out in the presence of an appropriate base, e.g., sodium bicarbonate, in an appropriate solvent, e.g., methanol. As can be appreciated by one skilled in the art, the above reaction provides an example of a generalized approach wherein compounds similar in structure to the specific reactants above (compounds similar to compounds of type 1.3), can be substituted in the reaction to provide substituted small molecule modulators of microtubule function similar to Formula 1.4.

2. Route II

In one aspect, substituted small molecule modulators of microtubule function can be prepared as shown below.

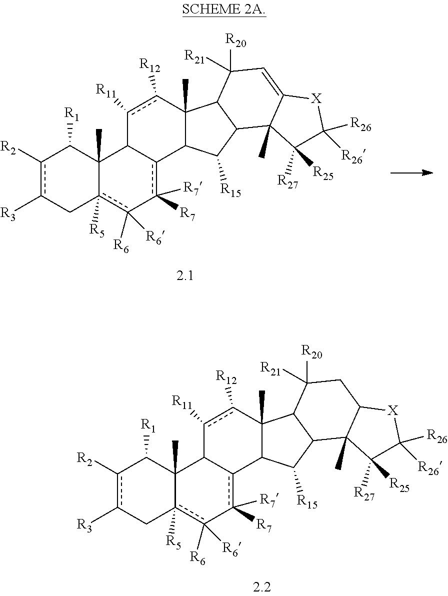

Compounds are represented in generic form, with substituents as noted in compound descriptions elsewhere herein. A more specific example is set forth below.

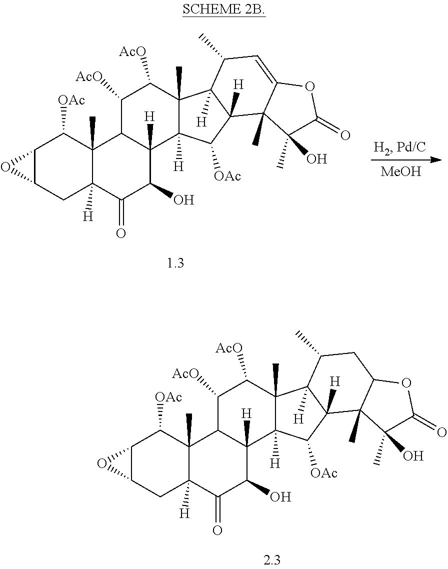

In one aspect, compounds of type 2.3, and similar compounds, can be prepared according to reaction Scheme 2B above. Thus, compounds of type 2.2 can be prepared by a hydrogenation reaction of an appropriate alkene, e.g., 2.1 as shown above. Appropriate alkenes are commercially available or prepared or isolated by methods known to one skilled in the art. The hydrogenation reaction is carried out in the presence of an appropriate hydride source, e.g., hydrogen gas, and an appropriate catalyst, e.g., palladium on carbon, in an appropriate solvent, e.g., methanol. As can be appreciated by one skilled in the art, the above reaction provides an example of a generalized approach wherein compounds similar in structure to the specific reactants above (compounds similar to compounds of type 1.3), can be substituted in the reaction to provide substituted small molecule modulators of microtubule function similar to Formula 2.3.

3. Route III

In one aspect, substituted small molecule modulators of microtubule function can be prepared as shown below.

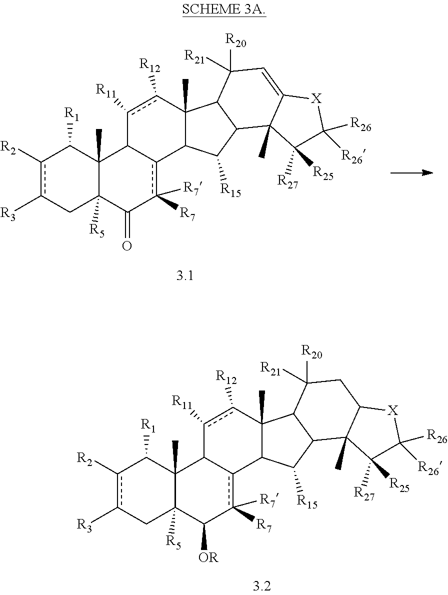

Compounds are represented in generic form, with substituents as noted in compound descriptions elsewhere herein and wherein R is hydrogen or acetyl. A more specific example is set forth below.

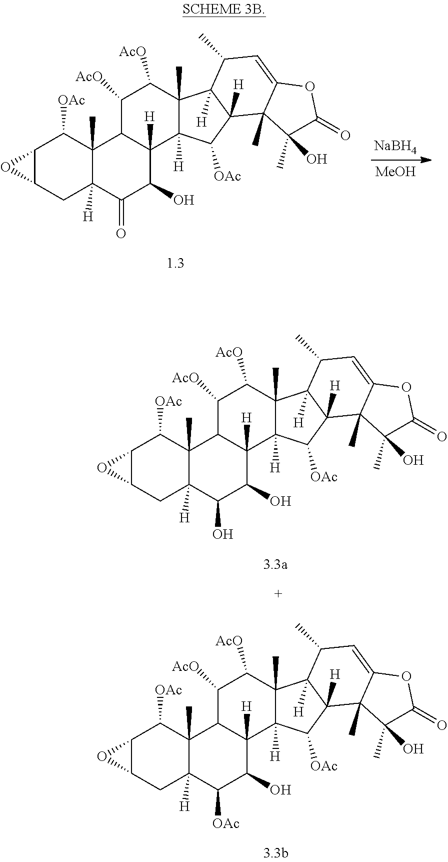

In one aspect, compounds of type 3.3a and 3.3b, and similar compounds, can be prepared according to reaction Scheme 3B above. Thus, compounds of type 3.2 can be prepared by reduction of an appropriate carbonyl analog, e.g., 3.1 as shown above. Appropriate carbonyls are commercially available or prepared or isolated by methods known to one skilled in the art. The reduction is carried out in the presence of an appropriate reducing agent, e.g., sodium borohydride, in an appropriate solvent, e.g., methanol. As can be appreciated by one skilled in the art, the above reaction provides an example of a generalized approach wherein compounds similar in structure to the specific reactants above (compounds similar to compounds of type 1.3), can be substituted in the reaction to provide substituted small molecule modulators of microtubule function similar to Formula 3.3a and 3.3b.

4. Route IV

In one aspect, substituted small molecule modulators of microtubule function can be prepared as shown below.

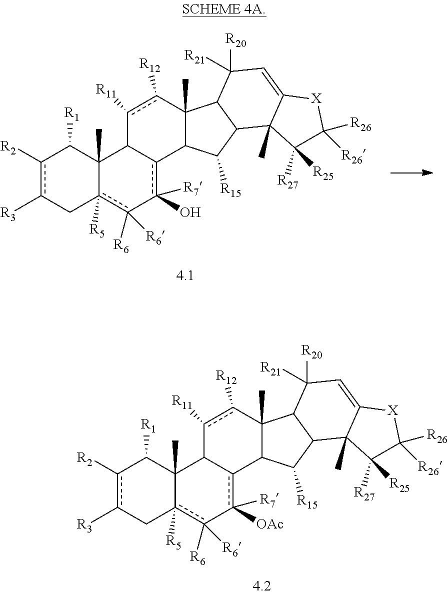

Compounds are represented in generic form, with substituents as noted in compound descriptions elsewhere herein. A more specific example is set forth below.

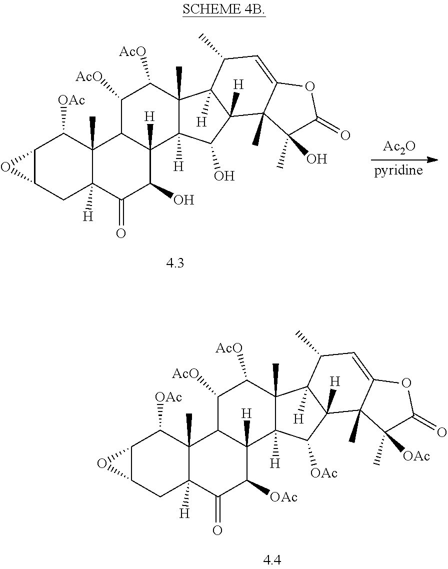

In one aspect, compounds of type 4.4, and similar compounds, can be prepared according to reaction Scheme 4B above. Thus, compounds of type 4.2 can be prepared by an acetylation reaction of an appropriate hydroxy analog, e.g., 4.1 as shown above. Appropriate hydroxy analogs are commercially available or prepared or isolated by methods known to one skilled in the art. The acetylation reaction is carried out in the presence of an appropriate acetyl agent, e.g., acetic anhydride, in an appropriate solvent, e.g., pyridine. As can be appreciated by one skilled in the art, the above reaction provides an example of a generalized approach wherein compounds similar in structure to the specific reactants above (compounds similar to compounds of type 4.3), can be substituted in the reaction to provide substituted small molecule modulators of microtubule function similar to Formula 4.4.

5. Route V

In one aspect, substituted small molecule modulators of microtubule function can be prepared as shown below.

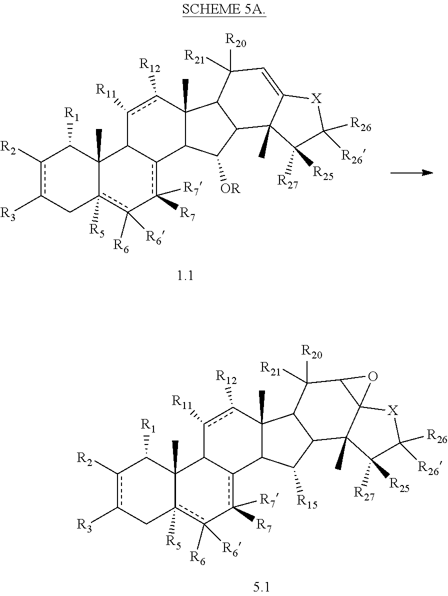

Compounds are represented in generic form, with substituents as noted in compound descriptions elsewhere herein. A more specific example is set forth below.

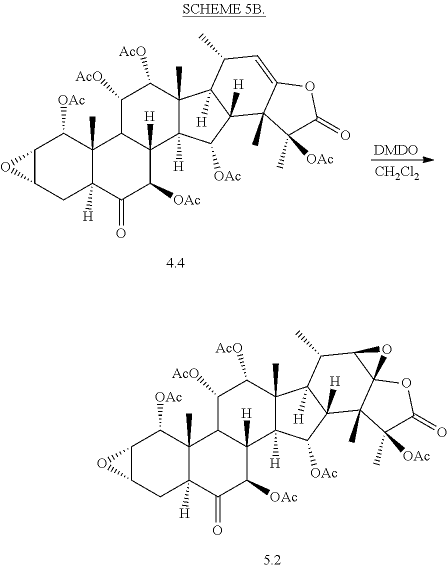

In one aspect, compounds of type 5.2, and similar compounds, can be prepared according to reaction Scheme 5B above. Thus, compounds of type 5.1 can be prepared by an epoxidation reaction of an appropriate alkene, e.g., 1.1 as shown above. Appropriate alkenes are commercially available or prepared or isolated by methods known to one skilled in the art. The epoxidation reaction is carried out in the presence of an appropriate epoxidizing agent, e.g., dimethyldioxirane, in an appropriate solvent, e.g., dichloromethane. As can be appreciated by one skilled in the art, the above reaction provides an example of a generalized approach wherein compounds similar in structure to the specific reactants above (compounds similar to compounds of type 4.4), can be substituted in the reaction to provide substituted small molecule modulators of microtubule function similar to Formula 5.2.

6. Route VI

In one aspect, substituted small molecule modulators of microtubule function can be prepared as shown below.

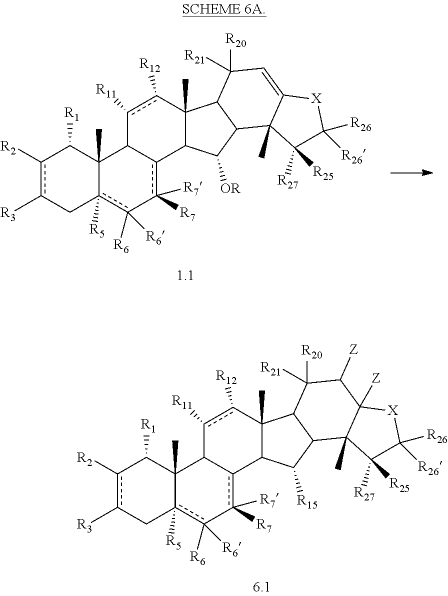

Compounds are represented in generic form, with substituents as noted in compound descriptions elsewhere herein and wherein each Z is independently halogen. A more specific example is set forth below.

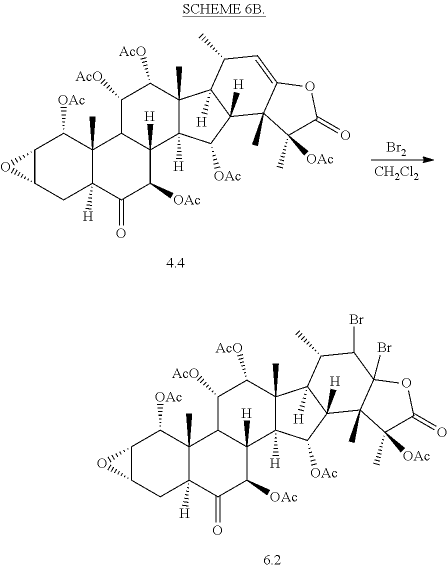

In one aspect, compounds of type 6.2, and similar compounds, can be prepared according to reaction Scheme 6B above. Thus, compounds of type 6.1 can be prepared by an addition reaction to an appropriate alkene, e.g., 1.1 as shown above. Appropriate alkenes are commercially available or prepared or isolated by methods known to one skilled in the art. The addition reaction is carried out in the presence of an appropriate halide source, e.g., bromine, in an appropriate solvent, e.g., dichloromethane. As can be appreciated by one skilled in the art, the above reaction provides an example of a generalized approach wherein compounds similar in structure to the specific reactants above (compounds similar to compounds of type 4.4), can be substituted in the reaction to provide substituted small molecule modulators of microtubule function similar to Formula 6.2.

7. Route VII

In one aspect, substituted small molecule modulators of microtubule function can be prepared as shown below.

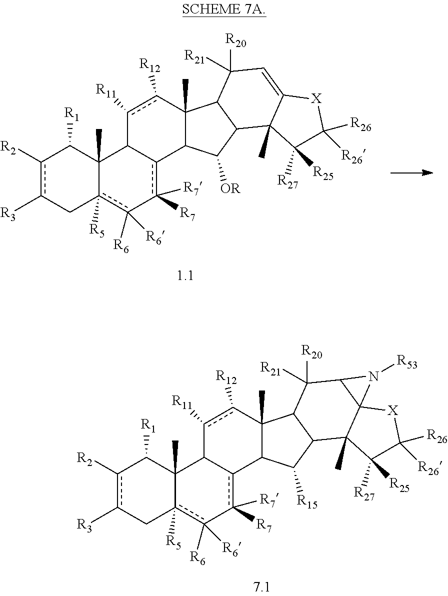

Compounds are represented in generic form, with substituents as noted in compound descriptions elsewhere herein. A more specific example is set forth below.

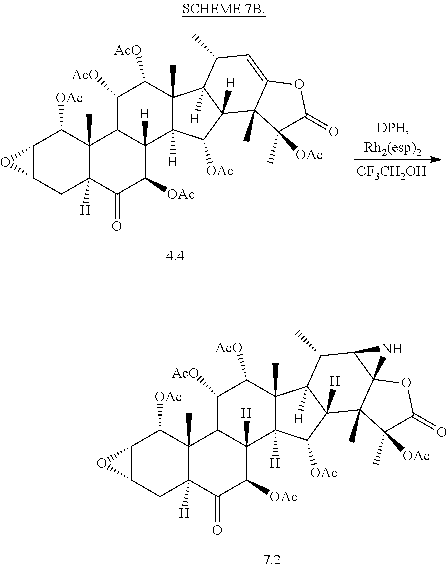

In one aspect, compounds of type 7.2, and similar compounds, can be prepared according to reaction Scheme 7B above. Thus, compounds of type 7.1 can be prepared by an aziridination reaction of an appropriate alkene, e.g., 1.1 as shown above. Appropriate alkenes are commercially available or prepared or isolated by methods known to one skilled in the art. The aziridination reaction is carried out in the presence of an appropriate aziridinating agent, e.g., O-(2,4-dinitrophenyl)hydroxylamine as shown above, and an appropriate catalyst, e.g., Bis[rhodium(α,α,α′,α′-tetramethyl-1,3-benzenedipropionic acid)] as shown above, in an appropriate solvent, e.g., 2,2,2-trifluoroethanol as shown above. As can be appreciated by one skilled in the art, the above reaction provides an example of a generalized approach wherein compounds similar in structure to the specific reactants above (compounds similar to compounds of type 4.4), can be substituted in the reaction to provide substituted small molecule modulators of microtubule function similar to Formula 7.2.

### D. PHARMACEUTICAL FORMULATIONS AND ROUTES OF ADMINISTRATION

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render agents stable and allow for uptake by target cells. Aqueous compositions of the present invention comprise an effective amount of the compounds, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. Such routes include oral, nasal, buccal, rectal, vaginal or topical route. Alternatively, administration may be by orthotopic, dermal, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the compounds of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### E. PROLIFERATIVE DISEASES

The present invention also involves, in one embodiment, the treatment of a hyperproliferative mammalian cell including a cancer cell. It is contemplated that a wide variety of tumors may be treated using taccalonolide therapy, including cancers of the brain, lung, liver, spleen, kidney, lymph node, pancreas, small intestine, blood cells, colon, stomach, breast, endometrium, prostate, testicle, ovary, uterus, skin, head and neck, esophagus, bone marrow, blood or other tissue. Other mammalian cells exhibiting a hyperproliferative phenotype including vascular or skin epidermal cells may be treated with a taccalonolide therapy.

It is not necessary that the cell be killed or induced to undergo normal cell death or “apoptosis.” Rather, to accomplish a meaningful treatment, all that is required is that the growth be slowed to some degree. It may be that the cell growth is completely blocked, however, or that some regression is achieved. Clinical terminology such as “remission” and “reduction of tumor” burden also are contemplated given their normal usage. Also, rendering a non-resectable tumor resectable may also be a useful clinical endpoint. Even the elongation of patient life, or reduction of patient discomfort (improving quality of life) is a goal of the present invention and thus helps define treatment.

### F. TREATMENT METHODS

Compounds that stabilize microtubules are generally useful as anti-cancer compounds and in the treatment of vascular diseases lining vascular stents. They can be administered to mammalian subjects (e.g., human patients) alone or in conjunction with other drugs that treat cancer or other hyperproliferative diseases.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2, 3, 4, 6, 8, 10, 20, 50, 100, 150, or more times). Encapsulation of the taccalonolide in a suitable delivery vehicle (e.g., polymeric microparticles or implantable devices) may increase the efficiency of delivery, particularly for oral delivery.

1. Methods of Treating a Hyperproliferative Disorder

In various aspects, the compounds and compositions disclosed herein are useful for treating, preventing, ameliorating, controlling or reducing the risk of a variety of hyperproliferative disorders. Thus, in one aspect, disclosed are methods of treating a hyperproliferative disorder in a subject, the method comprising administering to the subject an effective amount of at least one disclosed compound or a pharmaceutically acceptable salt thereof.

In various aspects, the disclosed compounds can be used in combination with one or more other drugs in the treatment, prevention, control, amelioration, or reduction of risk of hyperproliferative disorders for which disclosed compounds or the other drugs can have utility, where the combination of the drugs together are safer or more effective than either drug alone. Such other drug(s) can be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and a disclosed compound is preferred. However, the combination therapy can also include therapies in which a disclosed compound and one or more other drugs are administered on different overlapping schedules. It is also contemplated that when used in combination with one or more other active ingredients, the disclosed compounds and the other active ingredients can be used in lower doses than when each is used singly. Accordingly, the pharmaceutical compositions include those that contain one or more other active ingredients, in addition to a compound of the present invention.

In a further aspect, the compound exhibits microtubule stabilization. In a still further aspect, the compound exhibits modulation of microtubule structure and function. In yet a further aspect, the compound exhibits inhibition of cancer cell proliferation.

In a further aspect, the compound exhibits inhibition of cancer cell proliferation with an IC₅₀ of about 0.001 μM to about 25 μM. In a still further aspect, the compound inhibition of cancer cell proliferation with an IC₅₀ of about 0.001 μM to about 15 μM. In yet a further aspect, the compound exhibits inhibition of cancer cell proliferation with an IC₅₀ of about 0.001 μM to about 10 μM. In an even further aspect, the compound exhibits inhibition of cancer cell proliferation with an IC₅₀ of from about 0.001 μM to about 5 μM. In a still further aspect, the compound exhibits inhibition cancer cell proliferation with an IC₅₀ of from about 0.001 μM to about 1 μM. In yet a further aspect, the compound exhibits inhibition of cancer cell proliferation with an IC₅₀ of from about 0.001 μM to about 0.5 μM. In an even further aspect, the compound exhibits inhibition of cancer cell proliferation with an IC₅₀ of from about 0.001 μM to about 0.1 μM. In a still further aspect, the compound exhibits inhibition of cancer cell proliferation with an IC₅₀ of about 0.001 μM to about 0.05 μM. In yet a further aspect, the compound exhibits inhibition of cancer cell proliferation with an IC₅₀ of about 0.001 μM to about 0.01 μM. In an even further aspect, the compound exhibits inhibition of cancer cell proliferation with an IC₅₀ of from about 0.001 μM to about 0.005 μM. In a still further aspect, the compound exhibits inhibition of cancer cell proliferation with an IC₅₀ of about 0.005 μM to about 25 μM. In yet a further aspect, the compound exhibits inhibition of cancer cell proliferation with an IC₅₀ of about 0.01 μM to about 25 μM. In an even further aspect, the compound exhibits inhibition of cancer cell proliferation with an IC₅₀ of about 0.05 μM to about 25 μM. In a still further aspect, the compound exhibits inhibition of cancer cell proliferation with an IC₅₀ of about 0.1 μM to about 25 μM. In yet a further aspect, the compound exhibits inhibition of cancer cell proliferation with an IC₅₀ of from about 0.5 μM to about 25 μM. In an even further aspect, the compound exhibits inhibition of cancer cell proliferation with an IC₅₀ of about 1 μM to about 25 μM. In a still further aspect, the compound exhibits inhibition of cancer cell proliferation with an IC₅₀ of from about 5 μM to about 25 μM. In yet a further aspect, the compound exhibits inhibition of cancer cell proliferation with an IC₅₀ of about 10 μM to about 25 μM. In an even further aspect, the compound exhibits inhibition of cancer cell prolieration with an IC₅₀ of from about 15 μM to about 25 μM.

In a further aspect, the subject is a mammal. In a still further aspect, the mammal is human.

In a further aspect, the subject has been diagnosed with a need for treatment of the hyperproliferative disorder prior to the administering step. In a still further aspect, the subject is at risk for developing the disorder prior to the administering step.

In a further aspect, the method further comprises identifying a subject at risk for developing the disorder prior to the administering step.

2. Methods of Modulating Microtubule Function in at Least One Cell

In one aspect, disclosed are methods of modulating microtubule function leading to antiproliferative effects in at least one cell, the method comprising the step of contacting the at least one cell with an effective amount of at least one disclosed compound, or a pharmaceutically acceptable salt thereof. In a further aspect, modulating is inhibiting.

In a further aspect, the cell is mammalian. In a still further aspect, the cell is human. In yet a further aspect, the cell has been isolated from a mammal prior to the contacting step.

In a further aspect, contacting is via administration to a mammal. In a still further aspect, the mammal has been diagnosed with a need for treatment of a hyperproliferative disorder prior to the administering step.

In a further aspect, modulating is inhibiting microtubule function.

### G. STENTS

The present compounds may also be used as a coating on or impregnated into a stent. The anti-proliferative capacity of these compounds may find advantageous application in the treatment of vascular stenosis occurring subsequent to treatments involving stent placement.

A particular type of stent is a coronary stent. Coronary stents are effectively tubes placed in the coronary arteries to keep the arteries open in the treatment of coronary heart disease. It is often used in a procedure called percutaneous coronary intervention (PCI). Stents reduce chest pain and have been shown to improve survivability in the event of an acute myocardial infarction, but may suffer from restenosis, where the stent itself serves as a platform for narrowing the artery. The compounds of the present invention would be utilized to prevent cell proliferation in and around the stent, thereby reducing or slowing restenosis. Similar stents and procedures are used in non-coronary vessels, e.g., in the legs in peripheral artery disease.

### H. COMBINATION THERAPIES

It is common in many fields of medicine to treat hyperproliferative diseases including cancer with multiple therapeutic modalities, often called “combination therapies.” To treat hyperproliferative diseases using the methods and compositions of the present invention, one would generally contact a target cell or subject with a taccalonolide according to the present invention and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes a taccalonolide according to the present invention and the other includes the other agent.

Alternatively, a taccalonolide according to the present invention may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either a taccalonolide according to the present invention or the other therapy will be desired. Various combinations may be employed, where the taccalonolide according to the present invention is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. The skilled artisan is directed to “Remingtons Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Agents or factors suitable for use in a combined therapy include any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic” or “genotoxic agents,” are intended to be of use in the combined treatment methods disclosed herein. In treating cancer according to the invention, one would contact the tumor cells with an agent in addition to the expression construct. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition.

Various classes of chemotherapeutic agents are contemplated for use with in combination with taccalonolides of the present invention. For example, selective estrogen receptor antagonists; (“SERMs”), such as tamoxifen, 4-hydroxy tamoxifen (Nolvadex), fulvestrant (Falsodex), raloxifene (Evista); aromatase inhibitors including anastrozole (Arimidex), exemestane (Aromasin) and letrozole (Femara); antiandrogens including flutamide (Eulexin) and bicalutamide (Casodex).

Chemotherapeutic agents contemplated to be of use, include, e.g., camptothecin, actinomycin-D, mitomycin C. The invention also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a taccalonolide, as described above.

Heat shock protein 90 is a regulatory protein found in many eukaryotic cells. HSP90 inhibitors have been shown to be useful in the treatment of cancer. Such inhibitors include geldanamycin, 17-(Allylamino)-17-demethoxygeldanamycin, PU-H71 and Rifabutin.

Agents that directly cross-link DNA or form adducts are also envisaged. Agents such as cisplatin, carboplatin and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include doxorubicin (Adriamycin), etoposide, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the semi-synthesis of material derived from plants of the genus Taxus, and include paclitaxel, docetaxel and cabazitaxel. Other microtubule inhibitors include the epothilones, Vinca alkaloids or eribulin (Havalin).

mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in combination cancer therapy in accordance with the present invention.

Another possible combination therapy uses TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used. Other antimetabolites include methotrexate, premetrexed, 6-mercaptopurine, dacarbazine, fludarabine, capecitabine, gemcitabine and decitabine.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the local or regional delivery of a taccalonolide according to the present invention to patients with cancer will be a very efficient method for treating the clinical disease. Similarly, the chemo- or radiotherapy may be directed to a particular, affected region of the subject's body. Alternatively, regional or systemic delivery of expression construct and/or the agent may be appropriate in certain circumstances, for example, where extensive metastasis has occurred.

In addition to combining a taccalonolide according to the present invention with chemo- and radiotherapies, it also is contemplated that combination with immunotherapy, hormone therapy, toxin therapy and surgery. In particular, one may employ targeted therapies such as bevacizumab (Avastin), cetuximab (Erbitux), imatinib (Gleevec), transtuzumab (Herceptin) and rituximab (Rituxan).

It also should be pointed out that any of the foregoing therapies may prove useful by themselves in treating cancer.

### I. METHODS OF USING THE COMPOUNDS AND COMPOSITIONS

Provided are methods of using of a disclosed composition or medicament. In one aspect, the method of use is directed to the treatment of a hyperproliferative disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above mentioned pathological conditions.

1. Manufacture of a Medicament

In one aspect, the invention relates to a method for the manufacture of a medicament for treating a hyperliferative disorder in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of microtubule disruption. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

2. Use of Compounds and Compositions

Also provided are the uses of the disclosed compounds and compositions. Thus, in one aspect, the invention relates to the uses of modulators of microtubule function.

In a further aspect, the invention relates to the use of a disclosed compound or product of a disclosed method in the manufacture of a medicament for the treatment of a hyperproliferative disorder.

In a further aspect, the use relates to a process for preparing a pharmaceutical composition comprising a therapeutically effective amount of a disclosed compound or a product of a disclosed method, and a pharmaceutically acceptable carrier, for use as a medicament.

In a further aspect, the use relates to a process for preparing a pharmaceutical composition comprising a therapeutically effective amount of a disclosed compound or a product of a disclosed method, wherein a pharmaceutically acceptable carrier is intimately mixed with a therapeutically effective amount of the disclosed compound or the product of a disclosed method.

In various aspects, the use relates to the treatment of a hyperproliferative disorder in a vertebrate animal. In a further aspect, the use relates to the treatment of a hyperproliferative disorder in a human subject.

It is understood that the disclosed uses can be employed in connection with the disclosed compounds, methods, compositions, and kits. In a further aspect, the invention relates to the use of a disclosed compound or composition of a medicament for the treatment of a hyperproliferative disorder in a mammal.

In a further aspect, the invention relates to the use of a disclosed compound or composition in the manufacture of a medicament for the treatment of a hyperproliferative disorder.

3. Kits

In one aspect, disclosed are kits comprising a disclosed compound and one or more of: (a) at least one agent known to treat a hyperproliferative disorder; and (b) instructions for treating a hyperproliferative disorder.

In various aspects, the agents and pharmaceutical compositions described herein can be provided in a kit. The kit can also include combinations of the agents and pharmaceutical compositions described herein.

In various aspects, the informational material can be descriptive, instructional, marketing or other material that relates to the methods described herein and/or to the use of the agents for the methods described herein. For example, the informational material may relate to the use of the agents herein to treat a subject who has, or who is at risk for developing, a disorder associated with abnormal proliferation. The kits can also include paraphernalia for administering the agents of this invention to a cell (in culture or in vivo) and/or for administering a cell to a patient.

In various aspects, the informational material can include instructions for administering the pharmaceutical composition and/or cell(s) in a suitable manner to treat a human, e.g., in a suitable dose, dosage form, or mode of administration (e.g., a dose, dosage form, or mode of administration described herein). In a further aspect, the informational material can include instructions to administer the pharmaceutical composition to a suitable subject, e.g., a human having, or at risk for developing, a hyperproliferative disorder.

In various aspects, the composition of the kit can include other ingredients, such as a solvent or buffer, a stabilizer, a preservative, a fragrance or other cosmetic ingredient. In such aspects, the kit can include instructions for admixing the agent and the other ingredients, or for using one or more compounds together with the other ingredients.

In a further aspect, the compound and the at least one agent known to treat a hyperproliferative disorder are co-formulated. In a still further aspect, the compound and the at least one agent known to treat a hyperproliferative disorder are co-packaged.

In a further aspect, the at least one agent known to treat a hyperproliferative disorder is a chemotherapeutic agent. Examples of chemotherapeutic agents include, but are not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In a further aspect, the kit further comprises a plurality of dosage forms, the plurality comprising one or more doses; wherein each dose comprises an effective amount of the compound and the at least one agent known to treat a hyperproliferative disorder. In a still further aspect, the effective amount is a therapeutically effective amount. In yet a further aspect, the effective amount is a prophylactically effective amount. In an even further aspect, each dose of the compound and at least one agent known to treat a hyperproliferative disorder are co-packaged. In a still further aspect, each dose of the compound and the at least one agent known to treat a hyperproliferative disorder are co-formulated.

4. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a hyperproliferative prior to the administering step. In some aspects of the disclosed methods, the subject has been identified with a need for treatment prior to the administering step. In one aspect, a subject can be treated prophylactically with a compound or composition disclosed herein, as discussed herein elsewhere.

a. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, Pa.). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, N.Y.).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

### J. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. Instrumentation

NMR spectra were recorded on a Bruker Avance 600 or 700 MHz instrument equipped with a cryogenically cooled probe. All spectra were measured and reported in ppm using the residual solvent (CDCl₃) as an internal standard. The HRMS was measured using a Thermo Scientific LTQ Orbitrap mass spectrometer. IR data were obtained on a Bruker Vector 22 with a Specac Golden Gate ATR sampler. The UV spectra were measured on a Varian Cary 5000 UV-Vis NIR spectrophotometer. TLC was performed on aluminum sheets (silica gel 60 F₂₅₄, Merck KGaA, Germany). HPLC was performed on a Waters Breeze HPLC system. LC/MS was conducted on a Waters Alliance 2695 HPLC module, 996 photodiode array detector, and Micromass Quattro triple quadrupole mass spectrometer equipped with ESI. The purities of all compounds were determined to be greater than 95% by LC/MS and NMR.

2. Plant Material

Tacca chantreiri and T. integrifolia plants were purchased from a commercial grower. The roots and rhizomes were collected from living plants and stored at −20° C. until lyophilized.

3. Extraction and Isolation of Taccalonolide Z

The roots and rhizomes of T. integrifolia (1445 g) were extracted using supercritical fluid CO₂ with methanol and nonpolar lipids were removed by hexane extraction. The material was further extracted with CH₂Cl₂ to yield 11.7 grams of extract. The CH₂Cl₂ extract was purified by silica gel flash chromatography followed by repeated normal phase HPLC to yield 13.1 mg of taccalonolide Z. Taccalonolide Z was obtained as a white powder. The proton NMR spectrum showed four acetyl signals at δ 2.16, 2.13, 2.00, 1.97, five methyl signals at δ 1.64 (s), 1.34 (s), 0.98 (s), 0.89 (d, J=7.2 Hz), 0.73 (s), five oxygenated methine signals at δ 5.53 (t, J=10.2 Hz), 5.23 (br), 5.22 (dd, J=9.6, 2.4 Hz), 4.85 (d, J=5.4 Hz), 4.73 (dd, J=10.2, 5.4 Hz), two epoxyl methine signals at δ 3.74 (t, J=4.5 Hz) and 3.61 (dt, J=4.2, 1.8 Hz),), one olefinic signal at δ 5.06 (d, J=1.2 Hz). All these proton NMR data are similar to those of taccalonolide A and indicated that taccalonolide Z is a taccalonolide type steroid. The molecular formula of C₃₆H₄₆O₁₅ was determined by HRMS of 719.2934 (calcl 719.2915), suggesting that taccalonolide Z has one more oxygen than taccalonolide A. In addition, three signals for hydroxyl groups were observed at δ 3.64 (s), 3.45 (d, J=5.4 Hz), and 2.58 (s), which is one more than taccalonolide A. The carbon-13 NMR showed 7 oxygenated carbon signals at δ 79.08, 78.74, 74.13, 74.06, 71.20, 71.17, 71.14, and confirmed one more hydroxyl group for taccalonolide Z as compared to taccalonolide A. The ³J HMBC correlation between the hydroxyl proton signal at δ 3.64 and the carbonyl carbon at δ 208.34 (C-6) suggested that the hydroxyl group is located at C-5. The configuration of this hydroxyl group was determined as α by the NOE correlations between 5-OH/H-7,9,4α. The other ¹H and ¹³C NMR data for taccalonolide Z is similar to those for taccalonolide A, thus, taccalonolide Z was determined as 5α-hydroxy-taccalonolide A and this was confirmed by 2D NMR data. A trivial name taccalonolide Z was given to this compound.

Taccalonolide Z: white powder; ESIMS: m/z 719.4 [M+H]⁺, 736.4 [M+NH₄]+⁺, 731.5 [M+Na]⁺; ¹H NMR: δ (ppm) 5.53 (t, J=9.8 Hz, H-15), 5.23 (br., H-12), 5.22 (dd, J=9.6, 2.4 Hz, H-11), 5.06 (d, J=1.5 Hz, H-22), 4.85 (d, J=5.4 Hz, H-1), 4.73 (dd, J=10.2, 5.1 Hz, H-7), 3.74 (t, J=4.5 Hz, H-2), 3.64 (s, 5-OH), 3.61 (m, H-3), 3.45 (d, J=5.2 Hz, 7—OH), 3.17 (t, J=11.6 Hz, H-9), 2.58 (s, 25-OH), 2.57 (dd, J=15.0, 1.6 Hz, H-4a), 2.52 (t, J=10.1 Hz, H-14), 2.42 (dd, J=13.4, 10.2 Hz, H-16), 2.23 (d, J=16.7 Hz, H-4b), 2.16 (s, 3H, 1-OAc), 2.15 (m, H-20), 2.13 (s, 3H, 12-OAc), 2.00 (s, 3H, 15-OAc), 1.97 (s, 3H, 11-OAc), 1.81 (dd, J=13.4, 9.8 Hz, H-17), 1.64 (s, 3H, H-27), 1.56 (q, J=10.8 Hz, H-8), 1.34 (s, 3H, H-28), 0.98 (s, 3H, H-18), 0.89 (d, 3H, J=7.2 Hz, H-21), 0.73 (s, 3H, H-19); ¹³C NMR: δ (ppm) 208.34 (C-6), 178.10 (C-26), 172.07 (15-OAc), 170.85 (11-OAc), 169.40 (1-OAc), 169.25 (12-OAc), 154.50 (C-23), 111.07 (C-22), 79.08 (C-5), 78.74 (C-25), 74.13 (C-12), 74.06 (C-1), 71.20 (C-15), 71.17 (C-7), 71.14 (C-11), 54.16 (C-14), 54.06 (C-3), 50.97 (C-16), 50.60 (C-2), 50.07 (C-24), 48.85 (C-17), 45.86 (C-10), 44.19 (C-8), 43.15 (C-13), 37.13 (C-9), 30.61 (C-20), 26.94 (C-4), 25.32 (C-28), 22.36 (15-OAc), 21.16 (11-OAc), 21.02 (12-OAc), 20.72 (1-OAc), 20.61 (C-27), 20.08 (C-21), 14.61 (C-19), 13.37 (C-18).

4. Extraction and Isolation of the Taccalonolides A, E, AA, T, and R

Dried and pulverized rhizomes (2.3 kg) of T. chantrieri were extracted in several batches using supercritical CO₂ with MeOH. The crude extracts were washed with hexanes and extracted with CH₂Cl₂. The CH₂Cl₂ extracts were subjected to silica gel flash chromatography and eluted with hexanes:isopropanol (82:18) to obtain the taccalonolide enriched fraction. This fraction (1.4 g) was further purified on a silica gel HPLC column and eluted with isooctane:isopropanol (81:19) to yield fractions 1-8. Taccalonolides A and E were obtained from fractions 2 and 4 respectively. Fraction-1 (33 mg) was separated on a C-18 HPLC column, eluting with a gradient of acetonitrile:H₂O from 30% to 80% over 40 minutes, to yield 1.2 mg of taccalonolide AA and 0.8 mg of taccalonolide T. Fraction-3 was purified on silica gel flash column and eluted with CH₂Cl₂:acetone 85:15 to yield taccalonolide R.

a. Taccalonolide AA

Taccalonolide AA was isolated as a white powder. The proton NMR spectrum of taccalonolide AA showed characteristics almost identical to taccalonolide Z, indicating a similar taccalonolide structure. Five acetyl signals at δ 2.20, 2.15, 2.14, 2.00, 1.98, five methyl signals at δ 1.64 (s), 1.34 (s), 1.04 (s), 0.91 (d, J=7.0 Hz), 0.72 (s), five acetoxylated methine signals at δ 5.72 (d, J=11.0 Hz), 5.55 (d, J=9.5 Hz), 5.25 (br), 5.23 (brd, J=11.0 Hz), 4.91 (d, J=5.0 Hz), two epoxyl methine signals at δ 3.72 (t, J=4.5 Hz) and 3.59 (br), one olefinic signal at δ 5.09 (br). Taccalonolide AA has one more acetyl signal than taccalonolide Z. The chemical shift of H-7 at δ 5.72 (d, J=11.0 Hz) was approximately 0.99 ppm down-field than that of taccalonolide Z, suggesting this additional acetyl group was located at 7-OH. An HMBC correlation between H-7 and a carbonyl carbon at δ 170.8 confirmed this assignment. The other ¹H, ¹³C and 2D NMR data are similar to 5, thus, the structure of taccalonolide AA was determined and a trivial name taccalonolide AA was assigned.

Taccalonolide AA: white powder; ESIMS: m/z 761.4 [M+H]⁺, 778.4 [M+NH₄]⁺, 783.5 [M+Na]⁺, 701.3 [M-OAc]⁺; ¹H NMR: δ (ppm) 5.73 (d, J=11.0 Hz, H-7), 5.55 (t, J=9.4 Hz, H-15), 5.25 (d, J=2.6 Hz, H-12), 5.23 (dd, J=11.7, 2.6 Hz, H-11), 5.09 (d, J=1.4 Hz, H-21), 4.91 (d, J=5.5 Hz, H-1), 3.72 (t, J=4.5 Hz, H-2), 3.61 (s, 5-OH), 3.59 (m, H-3), 3.30 (t, J=11.4 Hz, H-9), 2.63 (t, J=10.0 Hz, H-14), 2.62 (s, 25-OH), 2.56 (brd, J=14.5 Hz, H-4a), 2.43 (dd, J=13.4, 9.8 Hz, H-16), 2.20 (s, 3H, 1-OAc), 2.19 (m, H-4b), 2.17 (m, H-20), 2.16 (s, 3H, 11-OAc), 2.15 (s, 3H, 12-OAc), 2.03 (q, J=11.0 Hz, H-8), 2.00 (s, 3H, 7-OAc), 1.98 (s, 3H, 15-OAc), 1.65 (s, 3H, H-27), 1.33 (s, 3H, H-28), 1.04 (s, 3H, H-18), 0.92 (s, 3H, H-21), 0.73 (s, 3H, H-18); ¹³C NMR: δ (ppm) 201.65 (C-6), 178.04 (C-25), 172.10 (15-OAc), 170.88 (11-OAc), 170.76 (7-OAc), 169.51 (1-OAc), 169.33 (12-OAc), 154.34 (C-23), 111.33 (C-22), 79.76 (C-5), 79.10 (C-26), 74.31 (C-7), 74.26 (C-1), 73.99 (C-12), 71.54 (11), 71.22 (C-15), 54.34 (14), 54.22 (C-3), 51.60 (C-16), 50.60 (C-2), 50.26 (C-24), 48.66 (C-17), 45.64 (C-10), 43.61 (C-13), 39.48 (C-8), 38.57 (C-9), 30.75 (C-20), 26.78 (C-4), 25.37 (C-28), 22.79 (15-OAc), 21.27 (7-OAc), 21.23 (12-OAc), 21.19 (11-OAc), 20.97 (1-OAc), 20.68 (C-21), 20.21 (C-27), 14.88 (C-19), 13.74 (C-18).

5. Extraction and Isolation of Taccalonolides A, B, AC, AD, AE, and AF

The roots and rhizomes of Tacca plantaginea were extracted with ethanol. The extract was subjected to silica gel column chromatography to generate a taccalonolide A fraction. This fraction (372.02 mg) was separated by column chromatography (Biotage) using HP silica and eluted with a gradient of CHCl₃:acetone yielding ten fractions. Taccalonolide B (5.95 mg) was obtained from fraction 4. Fraction 5 (252.92 mg) was subjected to HPLC purification and eluted with a gradient of acetonitrile:H₂O, yielding taccalonolide A, B and AE. Fraction 7 (20.51 mg) was purified using the same procedure yielding taccalonolide A (12.21 mg), B (0.33 mg), AE (1.39 mg), AD (2.29 gm) and AF (0.69 mg). Fraction 9 (5.25 mg) afforded taccalonolide H1 (0.89 mg), AD (0.92 mg), AE (1.02 mg) and AF (0.28 mg) after HPLC purification.

a. Taccalonolide AC

ESIMS: 717 [M+H—H₂O]⁺, 752 [M+NH₄]⁺. ¹H NMR: δ (ppm) 5.71 (s, H-22), 5.49 (t, J=9.0 Hz, H-15), 5.29 (d, J=2.7 Hz, H-12), 5.27 (dd, J=12.0, 2.7 Hz, H-11), 4.77 (d, J=5.8 Hz, H-1), 4.03 (dd, J=10.6, 4.4 Hz, H-7), 3.86 (d, J=4.4 Hz, 7-OH), 3.49 (dd, J=5.6, 3.1 Hz, H-2), 3.38 (m), 2.78 (dd, J=10.8, 4.1 Hz, H-5), 2.75 (t, J=11.6 Hz, H-9), 2.62 (m, H-16),2.61 (s, 25-OH), 2.60 (m, H-17), 2.41 (t, J=10.4 Hz, H-14), 2.24 (m, H₂-4), 2.18 (s, 3H, 1-OAc), 2.10 (s, 3H, 12-OAc), 2.01 (s, 6H, 11, 15-OAc), 1.75 (m, H-8), 1.72 (s, 3H, H-27), 1.38 (s, 3H, H-21), 1.35 (s, 3H, H-28), 1.10 (s, 3H, H-18), 0.77 (s, 3H, H-19). ¹³C NMR: δ (ppm) 210.0 (C-6), 178.1 (C-26), 172.4 (15-OAc), 170.8 (11-OAc), 170.0 (12-OAc), 169.6 (1-OAc), 153.9 (C-23), 112.1 (C-22), 84.5 (C-20), 79.4 (C-25), 74.8 (C-7), 73.8 (C-12), 72.8 (C-1), 71.0 (C-15), 70.9 (C-11), 53.8 (C-14), 52.3 (C-3), 50.3 (C-24), 49.6 (C-2), 46.4 (C-17), 45.5 (C-16), 43.8 (C-13), 43.2 (C-8), 42.8 (C-10), 42.2 (C-5), 40.1 (C-9), 25. (C-28), 21.9 (11, 15-OAc), 21.7 (C-4), 21.2 (12-OAc), 20.6 (1-OAc), 20.6 (C-21), 20.4 (C-27), 15.2 (C-18), 13.0 (C-19).

b. Taccalonolide AD

ESIMS: 701 [M+H]⁺, 718 [M+NH₄]⁺, 723 [M+Na]⁺. ¹H NMR: δ (ppm) 6.26 (s, 6-OH), 5.74 (dd, J=9.7, 8.7 Hz, H-15), 5.46 (dd, J=11.3, 3.3 Hz, H-11), 5.35 (d, J=3.3 Hz, H-11), 5.10 (d, J=1.4 Hz, H-22), 4.95 (d, J=5.5 Hz, H-1), 3.56 (dd, J=5.5, 4.0 Hz, H-2), 3.42 (brt, J=3.8 Hz, H-3), 3.36 (d, J=19.8 Hz, H-4), 2.88 (t, J=12.2 Hz, H-9), 2.63 (dd, J=19.8, 4.2 Hz, H-4), 2.62 (d, J=12.0 Hz, H-8), 2.57 (s, 25-OH), 2.48 (m, H-13), 2.47 (m H-16), 2.24 (m, H-20), 2.15 (15-OAc), 2.13 (1-OAc), 2.08 (12-OAc), 2.02 (11-OAc), 1.77 (dd, J=13.6, 10.0 Hz, H-17), 1.61 (s, 3H, H-27), 1.34 (s, 3H, H-28), 1.22 (s, 3H, H-19), 1.04 (s, 3H, H-18), 0.97 (d, 3H, J=7.1 Hz, H-21). ¹³C NMR: δ (ppm) 190.3 (C-7), 178.6 (C-26), 172.5 (15-OAc), 170.6 (11-OAc), 169.7 (1-OAc), 169.4 912-OAc), 154.2 (C-23), 143.9 (C-6), 127.3 (C-5), 111.1 (C-22), 79.3 (C-25), 72.4 (C-12), 71.7 (C-1), 70.1 (C-15), 69.5 (C-11), 51.1 (C-16), 50.7 (C-24), 49.6 (C-3), 49.1 (C-14), 48.6 (C-2), 47.5 (C-17), 43.8 (C-13), 40.0 (C-8), 38.7 (C-10), 38.1 (C-9), 30.3 (C-20), 24.5 (C-28), 23.3 (C-4), 22.7 (15-OAc), 21.1 (11-)Ac), 20.5 (12-OAc), 20.3 (1-OAc), 20.0 (C-27), 19.9 (C-21), 16.7 (C-19), 12.7 (C-18).

c. Taccalonolide AE

ESIMS: 719 [M+H]⁺, 736 [M+NH₄]⁺, and 741 [M+Na]⁺. ¹H NMR: δ (ppm) 5.60 (t, J=10.1 Hz, H-15), 5.30 (dd, J=11.6, 2.9 Hz, H-11), 5.27 (d, J=2.9 Hz, H-12), 5.10 (d, J=2.1 Hz, H-22), 5.01 (s, 7-OH), 4.73 (d, J=6.0 Hz, H-1), 3.64 (s, 7-OH), 3.48 (t, J=5.6, 4.2 Hz, H-2), 3.38 (m, H-4), 3.30 (dd, J=10.7, 5.0 Hz, H-5), 2.89 (t, J=12.0 Hz, H-9), 2.66 (t, J=10.1 Hz, H-15), 2.66 (dd, J=11.0, 9.6 Hz, H-14), 2.59 (s, 25-OH), 2.46 (dd, J=13.2, 10.7 Hz, H-16), 2.21 (m, H-20), 2.18 (m, H-4), 2.19 (s, 1-OAc), 2.14 (s, 12-OAc), 2.07 9s, 15-OAc), 2.00 (s, 11-OAc), 1.85 (m H-17), 1.83 (m, H-8), 1.65 (s, 3H, H-27), 1.35 (s, 3H, H-28), 1.03 (s, 3H, H-18), 0.94 (d, 3H, J=7.0 Hz, H-21), 0.79 (s, 3H, H-19). ¹³C NMR: δ (ppm) 206.7 (C-6), 178.0 (C-26), 171.0 (15-OAc), 170.8 (11-OAc), 169.7 (1-OAc), 169.3 (12-OAc), 154.4 (C-23), 111.4 (C-22), 92.4 (C-7), 79.1 (C-25), 73.8 (C-12), 72.8 (C-1), 72.5 (C-15), 70.8 (C-11), 52.2 (C-3), 51.1 (C-16), 49.8 (C-24), 49.6 (C-2), 49.1 (C-17), 48.4 (C-14), 44.2 (C-8), 43.2 (C-13), 42.7 (C-10), 39.6 (C-5), 39.2 (C-9), 30.9 (C-20), 25.3 (C-28), 22.4 (15-OAc), 21.5 (C-4), 21.2 (11-oaC), 20.9 (12-oaC), 20.7 (C-27), 20.6 (1-OAc), 20.0 (C-21), 13.4 (C-18), 12.5 (C-18).

d. Taccalonolide AF

ESIMS: 719 [M+H]⁺, 736 [M+NH₄]⁺, and 741 [M+Na]⁺. ¹H NMR: δ (ppm) 5.52 (t, J=9.4 Hz, H-15), 5.28 (dd, J=11.4, 2.7 Hz, H-11), 5.20 (d, J=2.7 Hz, H-12), 4.74 (d, J=5.5 Hz, H-1), 3.98 (dd, J=11.0, 4.1 Hz, H-7), 3.85 (d, J=4.1 Hz, 7-OH), 3.48 (ddt, J=5.6, 3.5 Hz, H-1), 3.39 (m, H-3), 3.29 (s, H-22), 2.76 (m, H-5), 2.71 (t, J=11.0 Hz, H-9), 2.69 (s, 25-OH), 2.43 (dd, J=11.4, 9.0 Hz, H-14), 2.21 (m, H-4), 2.19 (s, 3H, 1-OAc), 2.16 (s, 3H, 12-OAc), 2.07 (m, H-16), 2.03 (t, J=9.6 Hz, H-17), 2.02 (s, 3H, 15-OAc), 2.00 (s, 3H, 11-OAc), 1.76 (s, 3H, H-27), 1.35 (s, 3H, H-28), 1.03 (d, J=7.9 Hz, 3H, H-21), 0.88 (s, 3H, H-18), 0.78 (s, 3H, H-19). ¹³C NMR: δ (ppm) 209.9 (C-6), 177.4 (C-26), 171.6 (15-OAc), 170.5 (11-OAc), 169.4 (1-OAc), 169.0 (12-OAc), 92.2 (C-23), 79.6 (C-25), 75.7 (C-7), 74.0 (C-12), 73.1 (C-1), 71.6 (C-15), 71.2 (C-11), 65.9 (C-22), 54.6 (C-14), 52.9 (C-3), 49.9 (C-2), 48.1 (C-16), 46.8 (C-24), 45.2 (C-17), 43.7 (C-13), 43.4 (C-8), 43.2 (C-10), 42.6 (C-5), 40.3 (C-9), 32.1 (C-20), 24.1 (C-27), 22.9 (15-OAc), 21.8 (C-4), 21.4 (11-OAc), 21.0 (12-OAc), 20.3 (1-OAc), 20.1 (C-28), 19.1 (C-21), 13.6 (C-18), 13.5 (C-19).

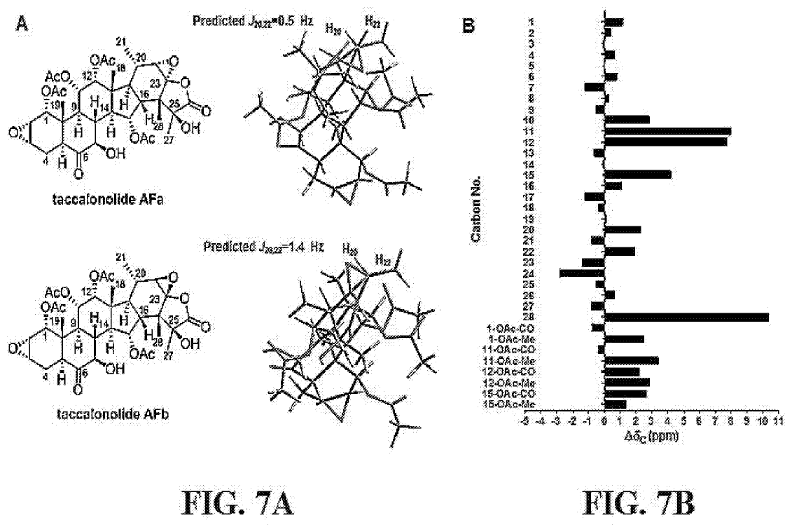

The absolute configuration of the synthetically-installed 22,23-epoxide in taccalonolide AF was deduced by interpretation of the small J_{H20,H22} coupling constant (see Li, J.; Risinger, A. L.; Peng, J.; Chen, Z.; Hu, L.; Mooberry, S. L., Potent taccalonolides, AF and AJ, inform significant structure-activity relationships and tubulin as the binding site of these microtubule stabilizers. J Am Chem Soc 2011, 133 (47), 19064-7). However, the high sterical selectivity of the epoxidation reaction on the 22,23-positions of natural taccalonolides has never been investigated. Recent efforts for generating taccalonolide analogues with substitutions on 22,23-positions encouraged the re-evaluation of the sterical selectivity of the 22,23-epoxidation. In order to confirm the absolute configuration of the 22,23-epoxide in taccalonolide AF, both the 22S,23S (AFa) and 22R,23R (AFb) isomers were subjected to computational DFT calculations. Conformational analyses were carried out using ComputeVOA™ v1.1. Geometry, frequency, and ¹³C NMR chemical shifts were calculated at the DFT level [OPBE functional 6-311+G(2d,p) basis set] with Gaussian '09 carried out in gas phase (see Du, L.; You, J.; Nicholas, K. M.; Cichewicz, R. H., Chemoreactive Natural Products that Afford Resistance Against Disparate Antibiotics and Toxins. Angew Chem Int Ed Engl 2016, 55 (13), 4220-5). For either taccalonolide AF isomer, only one lowest energy conformer was obtained as shown in FIG. 7A. The J_{H20,H22} coupling constants of both isomers were predicted to be small (0.5 Hz for AFa and 1.4 Hz for AFb) indicating it was not secure to determine the relative configuration of H-20 and H-22 solely based on the experimental J_{H20,H22} value. To provide additional evidence of the relative configuration of H-20 and H-22, the calculated ¹³C NMR chemical shifts of taccalonolides AFa and AFb were compared with experimental ¹³C NMR data of taccalonolide AF (FIG. 7B). For all the 36 carbons, a trend was observed that the calculated ¹³C NMR chemical shifts of taccalonolides AFb were generally more similar to the experimental values indicating the originally-assigned absolute configuration of 22,23-epoxide for taccalonolide AF should be revised.

Referring to FIG. 7A, structures of taccalonolide AFa (22S,23S) and taccalonolide AFb (22R,23R) and their computationally-optimized low-energy conformers are shown. The J_{H20,H22} coupling constants were predicted using the claculated dihedral angles of H₂₀ and H₂₂ (72° for AFa and 113° for AFb) in the Karplus equation.

Referring to FIG. 7B, a comparison of the DFT-calculated [OPBE/6-311+G(2d,p), gas phase] ¹³C NMR data of taccalonolides AFa and AFb with regard to their similarity to the experimental values of taccalonolides AF is shown. For a certain carbon, Δδ_{C}=|δ_{expt(AF)}−δ_{calc (AFa)}|−δ_{expt (AF)}−δ_{(calc (AFb)}|. When Δδ_{C}>0, the calculated carbon chemical shift of taccalonolide AFb is closer to the experimental value; when Δδ_{C}<0, the calculated carbon chemical shift of taccalonolide AFa is closer to the experimental value.

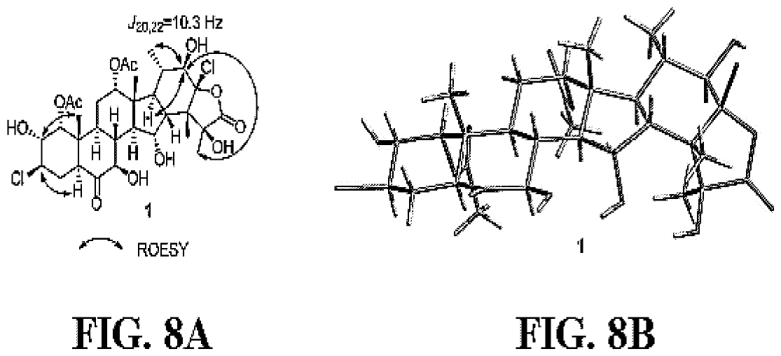

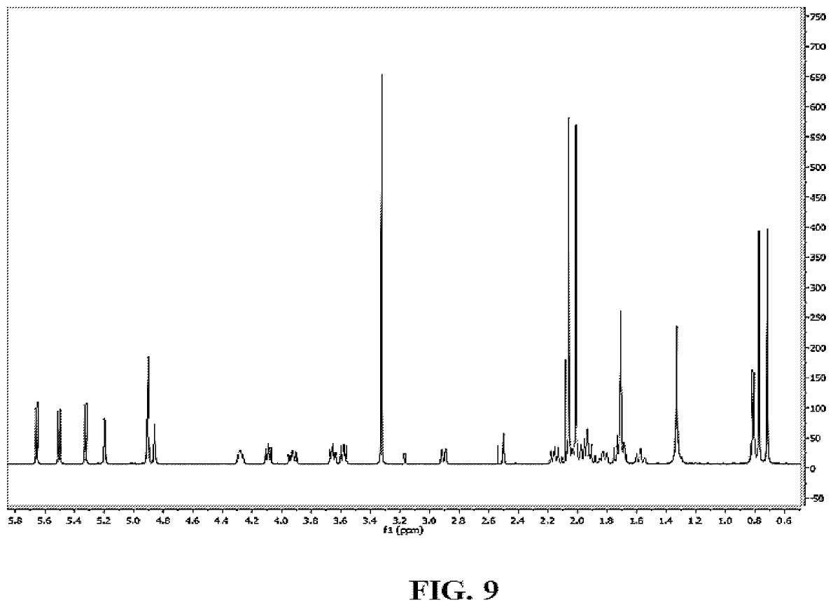

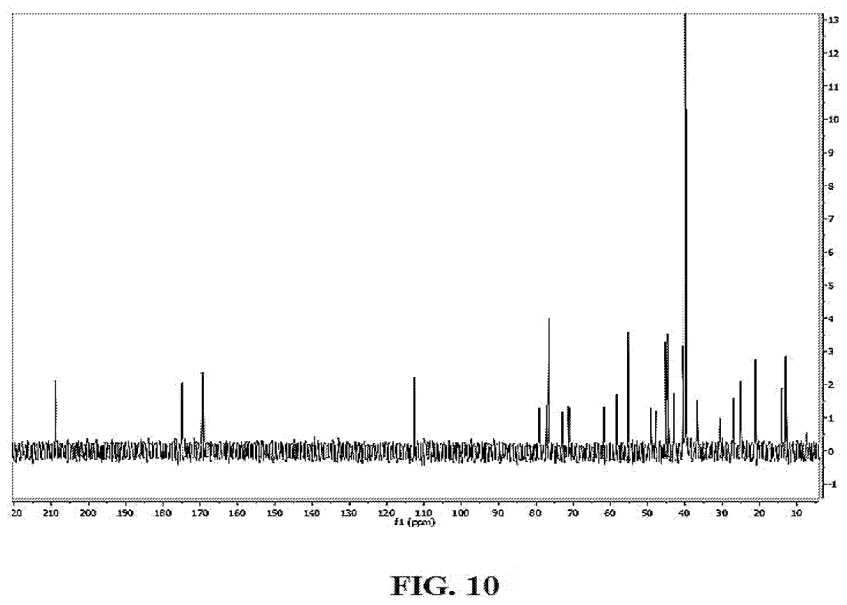

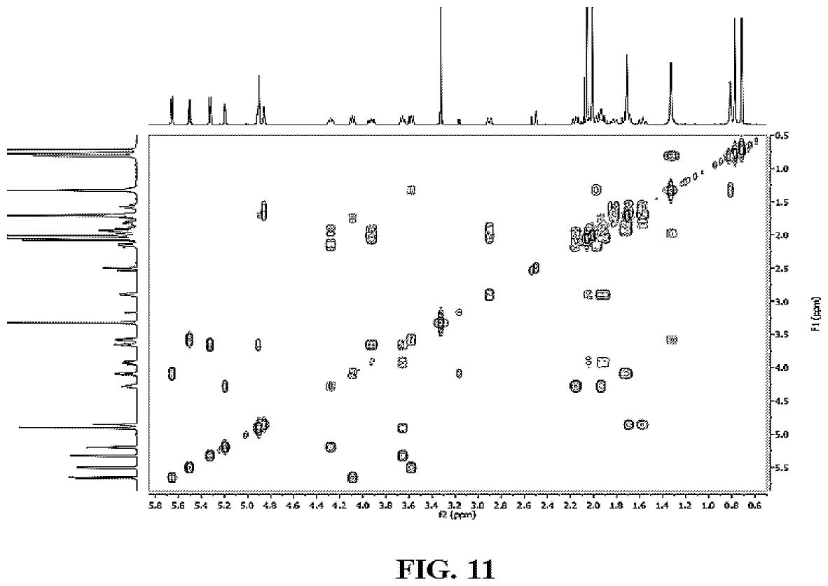

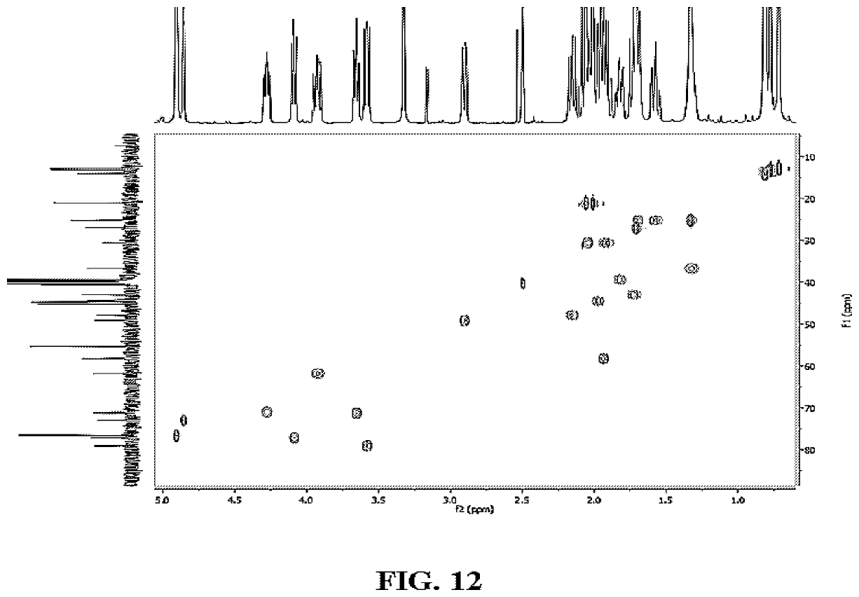

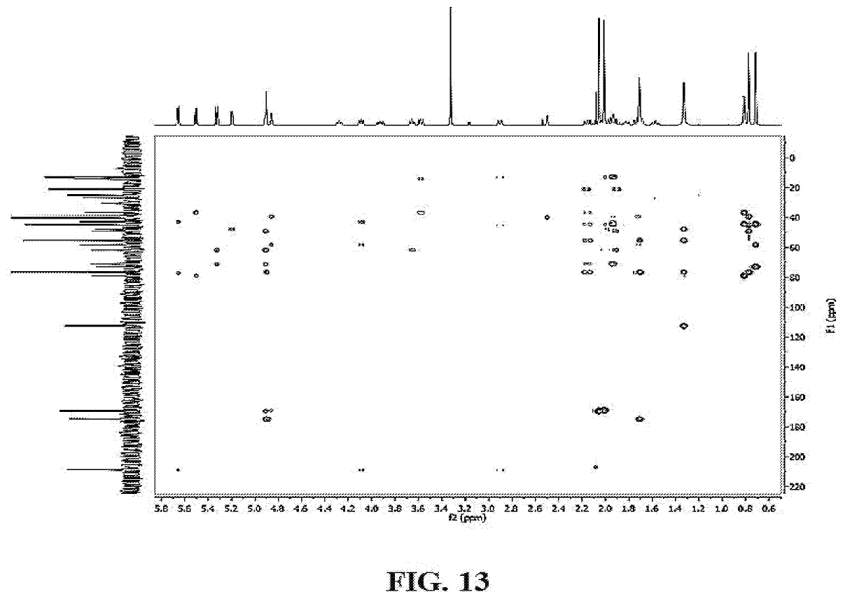

In order to confirm the revised absolute configuration of 22,23-epoxide, the taccalonolide N-epoxide, which was generated via standard epoxidation protocol using DMDO,³ was hydrolyzed by concentrated HCl (rt, stir, overnight) to yield a major epoxide-opening product 1 (Scheme 1). The large J_{H20,H22} coupling constant (10.3 Hz) indicated a trans configuration of H-20 and H-22 which was confirmed by the ROESY correlations between Me-21 and H-22, between H-17 and H-22, and between Me-27 and H-22 (FIG. 8A). Finally, the X-ray diffraction results of a single crystal of 1 secured the relative configuration of this compound (FIG. 8B). Thus, the absolute configuration of C-22 in 1 was deduced as R. Based on the well-established acidic opening mechanisms of epoxides, the 22-OH group in 1 should have retained the same orientation on the six-membered ring system as the 22,23-epoxide did in the structure of taccalonolide N-epoxide. Thus, the 22R,23R absolute configuration was confirmed for taccalonolide N-epoxide. In conclusion, the absolute configuration of 22,23-epoxides in taccalonolide N-epoxide, taccalonolide AF, and other 22,23-epoxidized taccalonolides is 22R,23R.

Referring to SCHEME 1, taccalonolide N-epoxide, the epoxidation product of taccalonolide N, was hydrolyzed in concentrated HCl (12 M) to yield compound 1.

Referring to FIG. 8A, key ROESY correlations and J_{H20,H22} coupling constant of 1 are shown.

Referring to FIG. 8B, X-ray diffraction structure of a single crystal of 1 is shown.

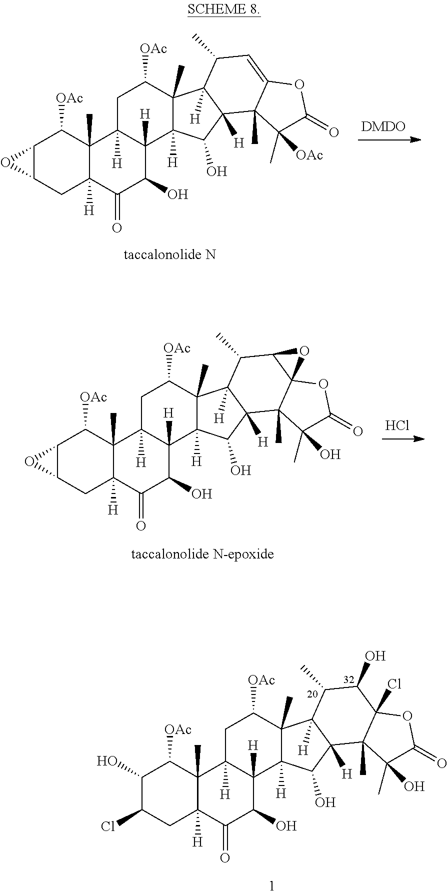

6. Extraction and Isolation of Taccalonolides B and AI

Dried and pulverized rhizomes of T. chantrieri were extracted in several batches using supercritical CO₂ with MeOH. The crude extracts were washed with hexanes and extracted with CH₂Cl₂. The CH₂Cl₂ extracts were subjected to silica gel flash chromatography and eluted with hexanes:isopropanol (82:18) to obtain the taccalonolide enriched fraction. This fraction was further purified on a silica gel HPLC column and eluted with isooctane:isopropanol (81:19) to yield fractions 1-8. Fraction 2 was hydrolyzed with 0.05 M sodium bicarbonate at room temperature for 40 h. The solution was stirred at room temperature for 44 h. The reaction solution was extracted with EtOAc and purified on HPLC to yield taccalonolide B as the major product and taccalonolide AI as a minor compound.

a. Taccalonolide AI

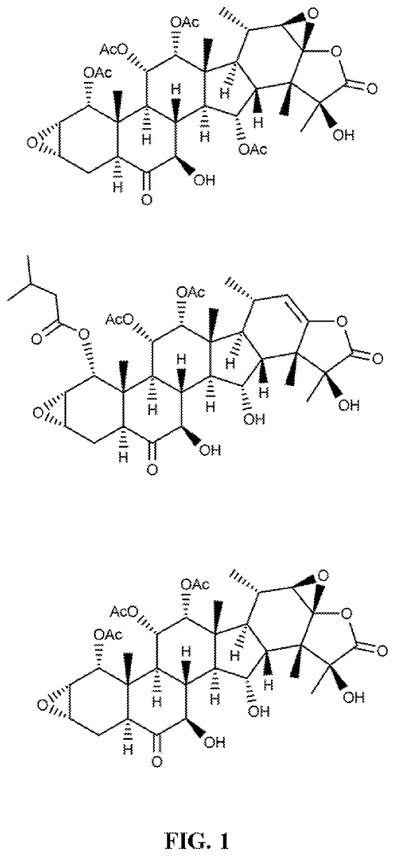

Taccalonolide AI was obtained as a white powder. The ESI-MS showed the protonated molecular ion at m/z 645.4 [M+H]⁺. The proton NMR spectrum showed only one acetyl signal at δ 2.08. This acetoxyl group was assigned to C-12 by the chemical shift of H-12 at 4.99 (t, J=2.7 Hz) and the HMBC correlation of this proton with the acetyl carbon. The chemical shift of H-15 at 4.38 (dt, J=11.2, 2.8 Hz) indicated a hydroxyl group at C-15. A 3-methylbutanoate was suggested by signals for two methyl group at 1.01 (d, J=6.1 Hz) and 1.00 (d, J=6.1 Hz) and confirmed by COSY and HSQC spectra. The correlations between H-1 at 4.59 and the carbonyl carbon at 171.8 located the 3-methylbutanoate at C-1. The other signals of taccalonolide AI are similar to taccalonolide N. Thus the structure of taccalonolide AI was determined as depicted. See FIG. 1.

Taccalonolide AI: white powder; ESIMS: m/z 645.4 [M+H]⁺, 662.3 [M+NH₄]⁺, 667.5 [M+Na]⁺, 599.3, 567.3, 557.2, 539.3, 521.2, 497.3; ¹H NMR (500 MHz, CDCl₃) δ 5.23 (d, J=2.6 Hz, 15-OH), 5.01 (br, H-22), 4.99 (t, J=2.7 Hz, H-12), 4.72 (s, 25-OH), 4.59 (d, J=5.2 Hz, H-1), 4.45 (br, 7-OH), 4.38 (dt, J=11.2, 2.8 Hz, H-15), 4.01 (d, J=10.3 Hz, H-7), 3.55 (t, J=5.8 Hz, H-2), 3.40 (br, H-3), 2.70 (dd, J=11.3, 4.5 Hz, H-5), 2.39 (dd, J=13.1, 10.9 Hz, H-6), 2.28 (dd, J=15.3, 4.3 Hz, H-4), 2.21 (m, H-20), 2.17 (m, H-4), 2.15 (m, H-9), 2.14 (m, CH₂ of 3-methylbutanoate), 2.13 (m, CH of 3-methylbutanoate), 2.11 (m. H-14), 2.08 (s, 12-OAc), 1.99 (dd, J=10.1, 13.5 Hz, H-17), 1.72 (m, H-8), 1.70 (m, H-11), 1.67 (s, H-27), 1.37 (s, H-28), 1.01 (d, J=6.1 Hz, CH₃ of 3-methylbutanoate), 1.00 (d, J=6.1 Hz, CH₃ of 3-methylbutanoate), 0.95 (d, J=7.2 Hz, H-21), 0.82 (s, H-18), 0.76 (s, H-19).

7. Extraction and Isolation of Taccalonolides AG and AH

The taccalonolides AG and AH were isolated from the roots of Tacca chantrieri. Freeze-dried material was ground to a fine powder and extracted with CO₂ and methanol using a supercritical fluid extractor. Non-polar lipids were removed by hexane extraction. The taccalonolides were further enriched by extraction with dichloromethane and water and the resultant fraction dried by evaporation. The crude taccalonolide extract was fractionated by flash chromatography on a silica column with hexanes and isopropanol. High performance liquid chromatography (HPLC) was used to separate the taccalonolides A and E. The HPLC fractions that eluted between A and E were combined and further fractionated by flash chromatography using a mixture of methylene chloride:acetone to generate 87 fractions. Fraction 29 was further separated by HPLC using a mixture of water:acetonitrile and a C18 Phenomenex large column. Fraction 18 contained an unresolvable mixture of taccalonolides AG and AH.

a. Taccalonolide AG

ESIMS: 703 [M+H]⁺, 720 [M+NH₄]⁺, and 725 [M+Na]⁺. ¹H NMR: δ (ppm) 5.51 (t, J=9.5 Hz, H-15), 5.11 (br, H-22), 5.03 (br, H-12), 4.61 (d, J=5.9 Hz, H-1), 3.89 (d, J=10.1 Hz, H-7), 3.82 (Br, 7-OH), 3.54 (t, J=4.5 Hz, H-2), 3.39 (m, H-3), 2.67 (dd, J=10.7, 6.0 Hz, H-5), 2.41 (dd, J=12.9, 9.6 Hz, H-16), 2.37 (t, J=9.4 Hz, H-14), 2.23 (m, H-4), 2.22 (m, H-20), 2.17 (m, CH₂ of isovalerate), 2.16 (m, H-9), 2.15 (m, CH of isovalerate), 2.11 (s, 15-OAc), 2.00 (s, 12-OAc), 1.96 (dd, 13.3, 3.8), 1.75 (m, H-11), 1.73 (m, H-8), 1.66 (s, 3H, H-27), 1.37 (s, 3H, H-27), 1.03 (d, 6H, J=4.8 Hz, CH₃ of isovalerate), 0.98 (d, J=6.5 Hz, H-21), 0.87 (s, 3H, H-18), 0.70 (s, 3H, H-19). ¹³C NMR: δ (ppm) 210.2 (C-6), 178.2 (C-26), 172.1 (15-OAc), 171.7 (1-isovalerate), 169.1 (12-OAc), 154.7 (C-23), 111.5 (C-22), 77.0 (C-7), 74.1 (C-12), 72.0 (C-15), 71.1 (C-1), 54.8 (C-14), 52.9 (C-3), 51.4 (C-16), 50.1 (C-24), 49.7 (C-2), 48.8 (C-17), 43.8 (C-5), 43.7 (C-8), 43.4 (CH₂ of isovalerate), 37.3 (CH of isovalerate), 31.0 (C-20), 25.9 (C-9), 25.8 (C-28), 25.2 (C-11), 22.8 (12-OAc), 22.5 (CH₃ of isovalerate), 21.6 (C-4), 21.3 (15-OAc), 21.1 (C-27), 19.7 (C-21), 13.4 (C-18), 13.2 (C-19).

8. Isolation of Taccalonolides AP, AQ, and AR

All the taccalonolides described in the literature were isolated from the roots and rhizomes of plants of the genus Tacca. In an attempt to identify new taccalonolides the petioles of T. chantrieri were investigated. The petioles were extracted three times with methanol and precipitated with methylene chloride. The supernatant was fractionated using silica flash chromatography with methylene chloride and methanol as solvents. 190 fractions were collected and combined based on their thin layer chromatography profiles. Fractions 85-89 were combined and subjected to another round of chromatography on a Biotage cartridge with methylene chloride and acetone as solvents. Two fractions were further purified by HPLC using a Phenomenex column with water and acetonitrile as solvents resulting in the pure taccalonolides AP and AQ in fractions 27 and 32 respectively. AR was purified by HPLC using fractions 90-91 from the initial flash purification and was found in the HPLC fraction 26.

9. Hydrolysis of Taccalonolides A, E, and Z to Yield Taccalonolides B, N, and AB, Respectively.

Taccalonolide A (40 mg) was dissolved in 4 mL of methanol and to this solution 8 mL of 0.05 M sodium bicarbonate was added. The solution was stirred at room temperature for 44 hours. The reaction solution was extracted with EtOAc and purified on HPLC to yield 25.8 mg of taccalonolide B. Taccalonolides N and AB were produced by hydrolysis of taccalonolides E and Z, respectively, using the same method. Taccalonolide AB was obtained as white powder. The LC/MS showed pseudomolecular ions at 677 [M+H]⁺, 694 [M+NH₄]⁺, and 699 [M+Na]⁺, indicating the loss of an acetyl group from taccalonolide Z. The proton NMR showed the chemical shift of H-15 of taccalonolide AB at δ 4.75 (ddd, J=3.5, 9.0, 11.6 Hz), which is shifted 0.78 ppm up-field than that of taccalonolide Z, suggesting the loss of acetyl group at 15-OH. The HMBC correlation between 15-OH (δ 4.94) and C-15 (δ 71.5) confirmed the assignment.

a. Taccalonolide AB

white powder; ESIMS: 677 [M+H]⁺, 694 [M+NH4]+, and 699 [M+Na]+. ¹H NMR: δ (ppm) 5.27 (dd, J=11.9, 2.1 Hz, H-11), 5.22 (d, J=2.1 Hz, H-12), 5.01 (br., H-21), 4.93 (d, J=3.6 Hz, 15-OH), 4.91 (dd, J=10.8, 4.6 Hz, H-7), 4.83 (d, J=5.4 Hz, H-1), 4.62 (br, 25-OH), 4.47 (ddd, J=11.1, 9.0, 3.4 Hz, H-15), 4.05 (d, J=4.5 Hz, 7-OH), 3.76 (t, J=4.5 Hz, H-2), 3.69 (s, 5-OH), 3.63 (m, H-3), 3.17 (t, J=11.6 Hz, H-9), 2.56 (brd, J=15.7 Hz, H-4a), 2.43 (dd, J=13.0, 11.0 Hz, H-16), 2.26 (m, J=16.8 Hz, H-4b), 2.24 (m, H-14),2.17 (s, 3H, 1-OAc), 2.15 (m, H-20), 2.14 (s, 3H, 12-OAc), 1.99 (s, 3H, 11-OAc), 1.86 (dd, J=13.2, 9.9 Hz, H-17), 1.69 (s, 3H, H-27), 1.64 (q, J=10.9 Hz, H-8), 1.37 (s, 3H, H-28), 0.97 (s, 3H, H-18), 0.89 (d, 3H, J=7.0 Hz, H-21), 0.78 (s, 3H, H-19); ¹³C NMR: δ (ppm) 207.23 (C-6), 175.35 (C-26), 171.12 (12-OAc), 169.64 (1-OAc), 169.51 (12-OAc), 154.90 (C-22), 110.43 (C-21), 79.10 (C-25), 78.75 (C-5), 74.41 (C-12), 74.12 (C-1), 72.04 (C-7), 71.46 (C-15), 70.89 (C-11), 57.57 (C-14), 54.12 (C-3), 51.04 (C-24), 50.79 (C-2), 50.28 (C-16), 48.19 (C-17), 46.06 (C-10), 44.06 (C-14), 43.82 (C-8), 36.66 (C-9), 31.17 (C-20), 27.07 (C-4), 25.62 (C-28), 21.99 (C-27), 21.35 (12-OAc), 21.14 (11-OAc), 20.83 (1-OAc), 20.30 (C-21), 14.70 (C-19), 13.44 (C-18).

10. Hydrolysis of Taccalonolide N Fraction and Isolation of Taccalonolides AK, AL, AM, and AN

The taccalonolide E fraction from the roots and rhizomes of Tacca chantrieri was hydrolyzed with mild base hydrolysis to produce predominantly taccalonolide N. This taccalonolide N enriched sample was further purified by HPLC using a C18 Phenomenex column and a solvent mixture of water and acetonitrile. Taccalonolide AN was found in fraction 9, taccalonolide AK in fraction 10, taccalonolide AL in fraction 24, and taccalonolide AM in fraction 22.

11. Hydrogenation of Taccalonolide A

6 mg of taccalonolide A was dissolved in MeOH and 0.5 mg of Pd—C was added. A stream of H₂ was bubbled into the solution using a balloon. The reaction was kept at room temperature for 6 h. The solution was filtered and dried to obtain dihydrotaccalonolide A.

12. Reduction of Taccalonolide A

6 mg of taccalonolide A was dissolved in 1 mL of MeOH and the solution was cooled on ice. NaBH₄ (3 mg) was added and stirred for 10 min. The solution was dried using miVac and the residue was extracted with CH₂Cl₂. The extract was dried and separated by HPLC to yield TA-NaBH₄-10 and TA-NaBH₄-12.

13. Acetylation of Taccalonolide B

Taccalonolide B (3 mg) was dissolved in 0.3 mL of acetic anhydride. To this solution, 0.3 mL of anhydrous pyridine was added and was kept at room temperature for 48 h. The reaction solution was dried in miVac and separated using C18 HPLC to yield taccalonolide A and TB-Ac-16.

14. Epoxidation of the Taccalonolides

Taccalonolide A (3.5 mg) was dissolved in 0.5 mL of methylene chloride and cooled to −20° C. with an ice salt bath. Dimethyldioxirane (0.1M, 75 μL) was added to the above solution. The temperature of the reaction was allowed to increase to room temperature and kept there until the reaction completed (approximately 4 h). The solvent was removed under vacuum and pure taccalonolide AF was obtained as white powder with 100% yield. The other epoxytaccalonolides were prepared using the same method. Taccalonolide AJ was produced using the above reaction with taccalonolide B as the starting material. This method is also applicable to epoxidate the crude taccalonolide extraction/fraction of Tacca spp. to produce the crude epoxytaccalonolide mixtures.

a. Taccalonolide AJ

Taccalonolide AJ was isolated as a white powder. The ESI-MS showed a protonated molecular ion at m/z 677.2 [M+H]⁺, which is one oxygen more than taccalonolide B. The proton NMR spectrum showed that H-22 was shifted from 5.00 ppm in taccalonolide B to 3.26 ppm, suggesting an epoxy group at C-22,23. No splitting of this signal requires the equatorial orientation of H-22, thus the epoxy group is a oriented. See FIG. 1.

Taccalonolide AJ: white powder; ESIMS: m/z 677.2 [M+H]⁺, 694.2 [M+NH₄]⁺, 699.2 [M+Na]⁺, 649.2 [M−H₂O+H]⁺, 631.3, 589.2, 571.3, 539.3, 529.2, 511.2, 479.2, 469.3; ¹H NMR (500 MHz, CDCl₃) δ 5.32 (dd, J=11.6, 2.5 Hz, H-11), 5.24 (d, J=3.1 Hz, H-12), 5.18 (d, J=2.4 Hz, 15-OH), 5.04 (s, 25-OH), 4.68 (d, J=5.5 Hz, H-1), 4.52 (br, 7-OH), 4.35 (dd, J=5.3 Hz, H-15), 4.17 (d, J=10.8 Hz, H-7), 3.50 (dd, J=4.5 Hz, H-2), 3.41 (br, H-3), 3.26 (s, H-22), 2.80 (dd, J=11.3, 4.3 Hz, H-5), 2.70 (t, J=11.5 Hz, H-9), 2.30-2.1 (m, H-4, 14, 16, 17), 2.17 (s, 1-OAc), 2.14 (s, 12-OAc), 1.99 (S, 11-OAc), 1.36 (s, 3H), 1.76 (s, H-27), 1.36 (s, H-28), 1.02 (d, J=7.9 Hz, H-21), 0.85 (s, H-18), 0.84 (s, H-18).

15. Cell Culture

The HeLa cervical cancer cell line, the SK-OV-3 ovarian cancer cell line and the PC-3 prostate cancer cell line were obtained from American Type Tissue Culture Collection (Manassas, Va.) and grown in Basal Media Eagle (BME) or RPMI 1640 medium (Invitrogen; Carlsbad, Calif.) supplemented with 10% fetal bovine serum (Hyclone; Logan, Utah) and 50 μg/ml gentamicin sulfate (Invitrogen). The P-glycoprotein expressing SK-OV-3/MDR-1-6/6 cell line and the βIII-tubulin expressing WTβIII cell line have been described previously (Risinger et al., 2008).

16. Inhibition of Cellular Proliferation and Initiation of Cytotoxicity

The antiproliferative and cytotoxic effects of the taccalonolides were evaluated using the SRB assay (Skehan et al., 1990, Boyd and Paull, 1995) as previously described (Tinley et al., 2003). The concentration of drug that causes 50% inhibition of cellular proliferation (IC₅₀) was calculated from the linear portion of the log dose response curve. The ability of the compounds to initiate cytotoxicity was also determined. Paclitaxel was included as a reference compound. The determination of IC₅₀ values was performed on taccalonolide material after NMR analysis and subsequent lyophilization. Ethanol or DMSO was used as the vehicle for all cellular studies.

17. Immunofluorescence

Cellular microtubules in interphase and mitotic HeLa cells were visualized using indirect immunofluorescence techniques as previously described (Tinley et al., 2003). Cells were treated for 18 h with vehicle, the taccalonolides or the positive control paclitaxel, fixed with methanol and microtubules visualized with a β-tubulin antibody. Representative images of interphase and mitotic cells were acquired using a Nikon Eclipse 80i fluorescence microscope and compiled using NIS Elements AR 3.0 software.

18. Flow Cytometry

HeLa cells were incubated for 18 h with vehicle, each taccalonolide or paclitaxel as a positive control. The cells were harvested and the DNA was stained with propidium iodide using Krishan's reagent (Krishan, 1975). Cellular DNA content was analyzed using a FACS Calibur flow cytometer (BD Biosciences). Data were plotted as propidium iodide intensity versus the number of events using ModFit LT 3.0 software (Verity Software, Topsham, Me.).

19. Microtubule Stabilization and Mitotic Arrest

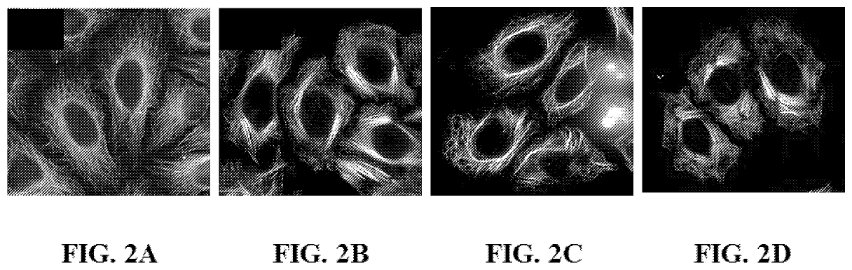

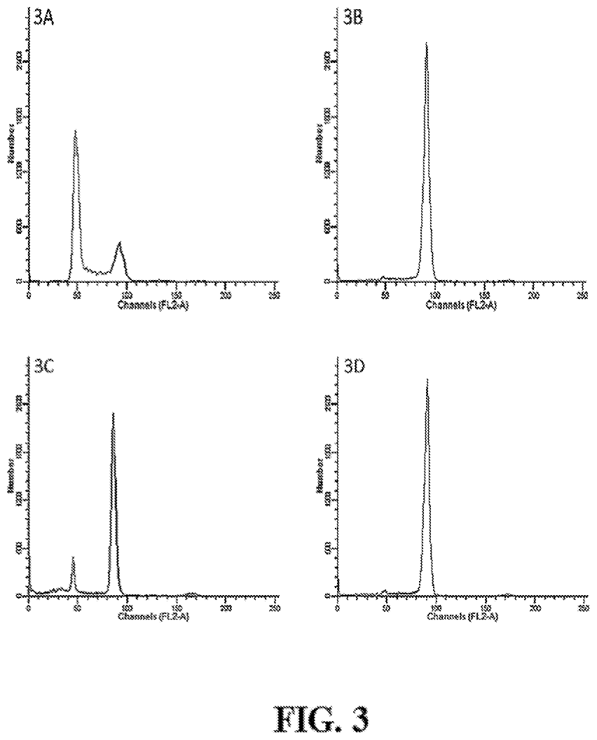

The ability of the newly isolated taccalonolides to cause bundling of interphase microtubules was evaluated in HeLa cells. Consistent with the effects of taccalonolides A and E, which were shown to exert interphase microtubule bundling in previous studies (Tinley et al., 2003), taccalonolides AF, AI, and AJ each caused the formation of thick bundled microtubule tufts typical of microtubule stabilizers including paclitaxel (FIG. 2A-D). Although microtubule stabilizers cause an increase in the density of interphase microtubules, the mechanism by which these agents inhibit the proliferation of cancer cells in vitro is widely accepted to be due to their ability to interrupt microtubule dynamics in mitosis, leading to mitotic arrest. The effect of the taccalonolides on mitotic progression was analyzed by flow cytometry. All taccalonolides caused an accumulation of cells in the G₂/M phase of the cell cycle with 4N DNA content (FIG. 3A-D). This accumulation is identical to the mitotic arrest that is observed after treatment of HeLa cells with paclitaxel (FIG. 3A-D). Recent data also suggests that the ability of microtubule stabilizers to interrupt cellular trafficking and metabolism in interphase cells also leads to the initiation of cell death (Reviewed in Komlodi-Pasztor, 2011).

Referring to FIG. 2A-D, HeLa cells were treated for 18 h with vehicle (FIG. 2A), 200 nM taccalonolide AF (FIG. 2B), 200 nM taccalonolide AI (FIG. 2C), or 70 nM taccalonolide AJ (FIG. 2D). Interphase microtubule structures were visualized by indirect immunofluorescence using a β-tubulin antibody.

Referring to FIG. 3A-D, HeLa cells were treated with vehicle (FIG. 3A), 125 nM taccalonolide AF (FIG. 3B), 200 nM taccalonolide AI (FIG. 3C), or 35 nM taccalonolide AJ (FIG. 3D) for 18 h and stained with Krishan's reagent. Cell cycle profile was analyzed by flow cytometry.

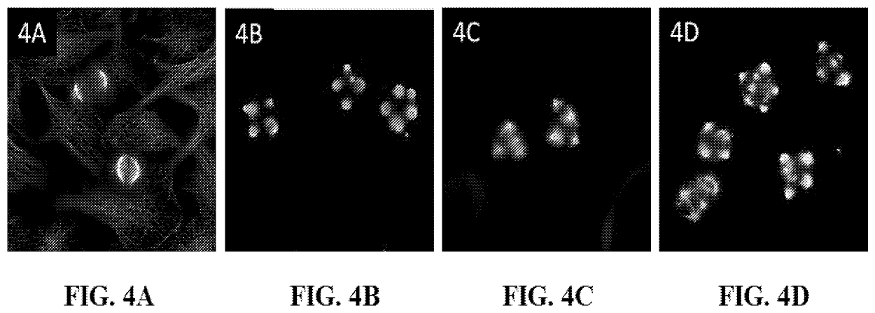

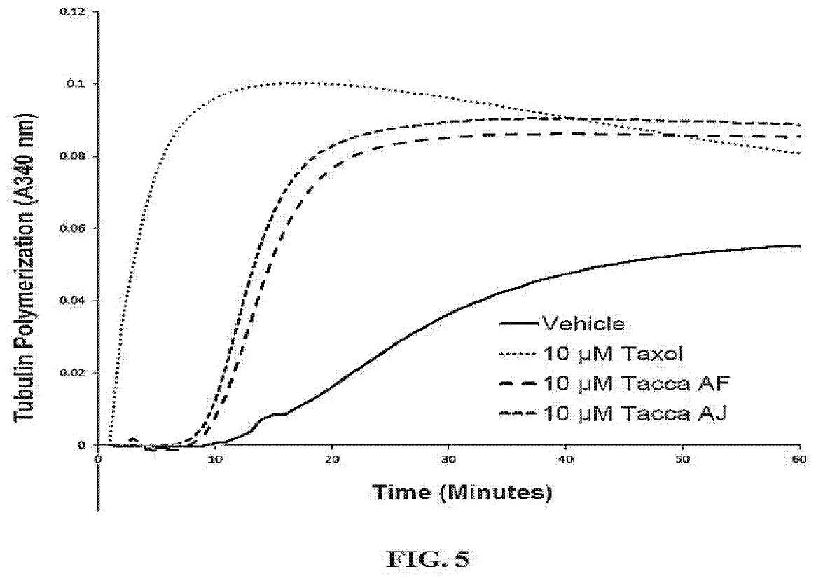

The effects of the taccalonolides on mitotic spindle structures were evaluated to test whether they caused mitotic spindle defects leading to cell cycle arrest. β-tubulin and DNA were visualized in HeLa cells by indirect immunofluorescence and DAPI staining, respectively. The majority of cells treated with each taccalonolide at the concentration that caused G₂/M accumulation were found to be in mitosis as evidenced by a “rounded up” cellular morphology and condensed DNA. These mitotic cells contained multiple abnormal mitotic spindles, which is another common effect of microtubule stabilizing agents (FIGS. 4A-D). These findings demonstrate that all taccalonolides, including AF, AI and AJ are microtubule stabilizers that cause mitotic arrest of cells with multiple abnormal mitotic spindles.

Referring to FIG. 4A-D, HeLa cells were treated for 18 h with vehicle (FIG. 4A), 125 nM taccalonolide AF (FIG. 4B), 200 nM taccalonolide AI (FIG. 4C), or 35 nM taccalonolide AJ (FIG. 4D). The microtubule structures in mitotic cells were visualized by indirect immunofluorescence using a β-tubulin antibody.

20. Antiproliferative Activities of the Taccalonolides

The antiproliferative potencies of the taccalonolides were evaluated in HeLa cells using the SRB assay. Several new taccalonolides with low nanomoloar potency were identified, see Table 1 and Table 2. The most potent taccalonolide is the newly synthesized taccalonolide AI-epo, with an IC₅₀ value of 0.73 nM (Table 1). This makes taccalonolide AI-epo the most potent taccalonolide identified thus far. Each of the taccalonolides tested also initiates cytotoxicity. This low nanomolar potency of some of the new taccalonolides is identical or superior to other naturally occurring microtubule stabilizers, including paclitaxel, the epothilones, laulimalide and peloruside A, in comparison to the taccalonolides A and E (Risinger et al., 2008).

TABLE 1

| | | Corresponding | IC₅₀ |
|---|---|---|---|
| Taccalonolide | IC₅₀ (nM) | Epoxide | (nM) |
| Taccalonolide A | 5,380 | Taccalonolide AF | 23 |
| Taccalonolide B | 3,120 | Taccalonolide AJ | 4.3 |
| Taccalonolide E | 39,500 | E-epo | 67 |
| Taccalonolide I | >10,000 | I-epo | 327 |
| Taccalonolide N | 8,500 | N-epo | 11 |
| Taccalonolide R | 13,144 | R-epo | 18 |
| Taccalonolide S | 9 | N/A | |
| Taccalonolide T | 335 | N/A | |
| Taccalonolide H2 | 730 | H2-epo | 37 |
| Taccalonolide Z | 120 | Z-epo | 21 |
| Taccalonolide AA | 32.3 | AA-epo | 15 |
| Taccalonolide AB | 2,767 | AB-epo | 5.0 |
| Taccalonolide AC | >50,000 | AC-epo | ~40 μM |
| Taccalonolide AD | 3,480 | AD-epo | 338 |
| Taccalonolide AE | 5,010 | AE-epo | 422 |
| Taccalonolide AG | 32 | | |
| (in mixture with AH) | | | |
| Taccalonolide AH | 158 | AH-epo | 7 |
| Taccalonolide AI | 47 | AI-epo | 0.73 |
| Taccalonolide AK | >30,000 | N/A | |
| Taccalonolide AL | 18,000 | AL-epo | 134 |
| Taccalonolide AM | 1,200 | AM-epo | 16 |
| Taccalonolide AN | 1,000 | AN-epo | 265 |
| Taccalonolide AO | >30,000 | NA | |
| Taccalonolide AP | >30,000 | AP-epo | 333 |
| Taccalonolide AQ | >30,000 | AQ-epo | 463 |
| Taccalonolide AR | >30,000 | AR-epo | 366 |
| Taccalonolide AS | >10,000 | AS-epo | ~25 μM |
| TA-NaBH4-12 | 7,500 | TA-NaBH4-12-epo | 131 |
| TA-NaBH4-10 | 20,000 | TA-NaBH4-10-epo | 235 |
| TB-AC-16 | 40,000 | TB-Ac-16-epo | 252 |

The IC₅₀ values of unnamed taccalonolides are indicated adjacent to the respective structures. The concentrations of drugs that caused a 50% inhibition of cellular proliferation (IC₅₀) were measured in HeLa cells using the SRB assay. N/A is not available.

TABLE 2

| Compound | |
|---|---|
| Name | Structure |
| Taccalonolide A | 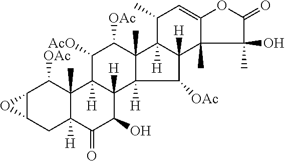 |
| Taccalonolide B | 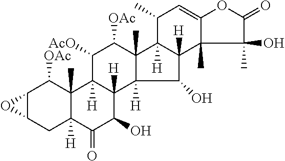 |
| Taccalonolide C | 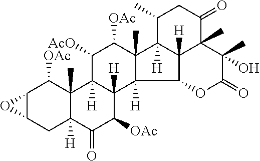 |
| Taccalonolide D | 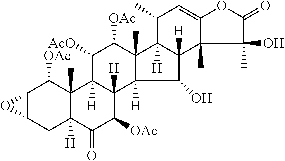 |
| Taccalonolide E | 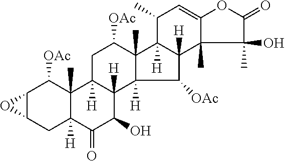 |
| Taccalonolide F | 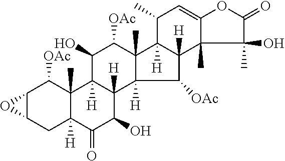 |
| Taccalonolide G | 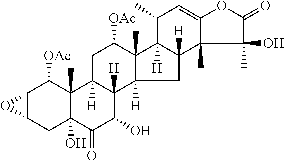 |
| Taccalonolide H | 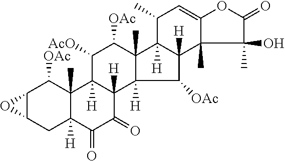 |
| Taccalonolide I | 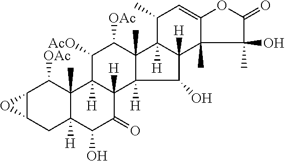 |
| Taccalonolide J | 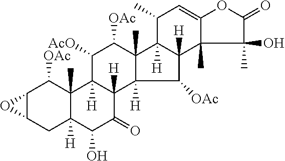 |
| Taccalonolide K | 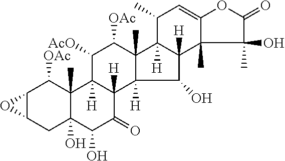 |
| Taccalonolide L | 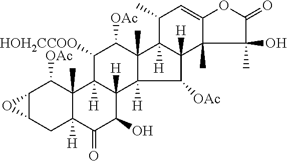 |
| Taccalonolide M | 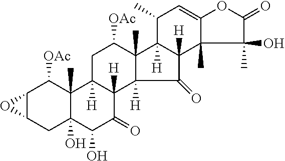 |
| Taccalonolide N | 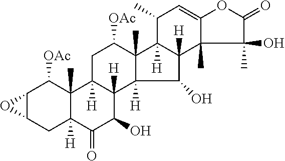 |
| Taccalonolide O | 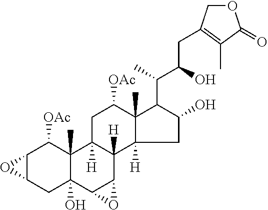 |
| Taccalonolide P | 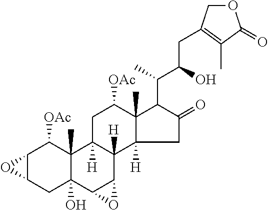 |
| Taccalonolide Q | 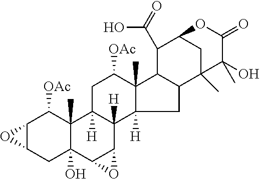 |
| Taccalonolide R | 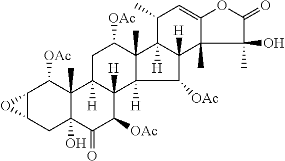 |
| Taccalonolide S | 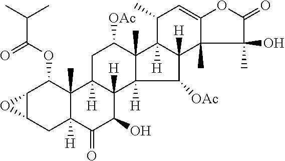 |
| Taccalonolide T | 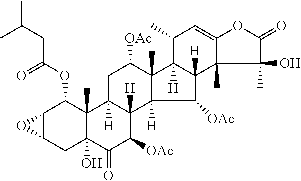 |
| Taccalonolide U | 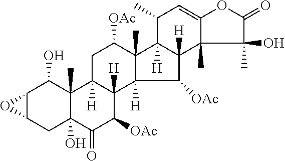 |
| Taccalonolide V | 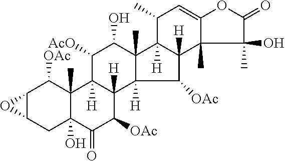 |
| Taccalonolide W | 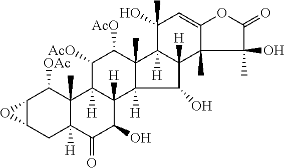 |
| Taccalonolide X | 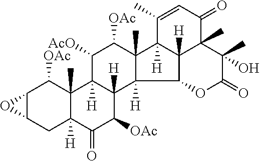 |
| Taccalonolide Y | 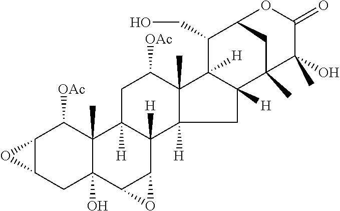 |

21. Tubulin Binding Activity of the Taccalonolides

The ability of these new potent taccalonolides to interact directly with tubulin was assessed by incubating purified porcine brain tubulin at a concentration of 2 mg/ml in the presence of 10% glycerol and 1 mM GTP, which allows for a baseline level of tubulin polymerization that can be followed turbidimetrically (FIG. 5). The rate and extent of tubulin polymerization is dramatically increased when 10 μM of taccalonolide AF or AJ is added to the tubulin polymerization reaction, which is similar to the effects of the known microtubule interacting drug paclitaxel in this assay (FIG. 5). This result indicates that these potent taccalonolides can interact with purified tubulin and/or microtubules to enhance their polymerization.

Referring to FIG. 5, 2 mg/ml porcine brain tubulin in 10% glycerol and 1 mM GTP was incubated at 37° C. in the presence of vehicle or 10 μM paclitaxel, taccalonolide AF or taccalonolide AJ. Tubulin polymerization was monitored by turbidity measurement at OD₃₄₀.

22. Antitumor Activity of Taccalonolide AF

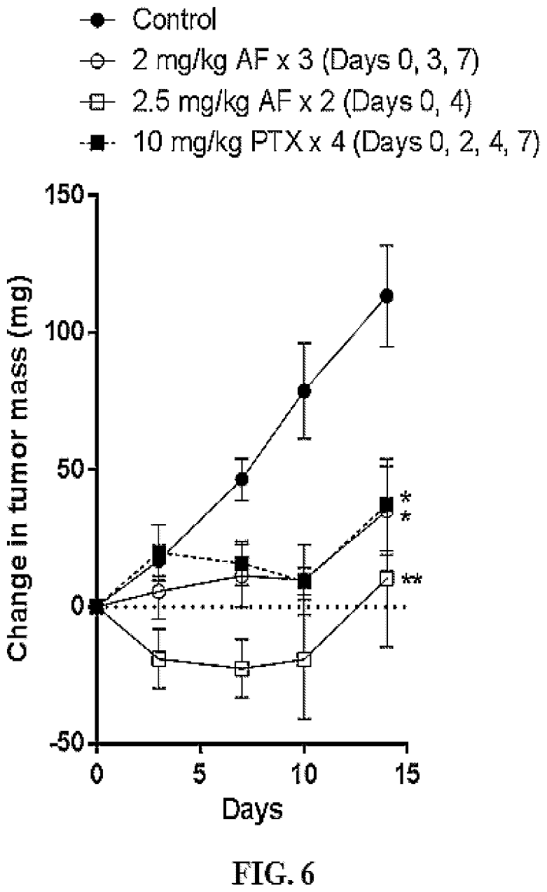

The ability of taccalonolide AF to inhibit the growth of the aggressive human breast tumor MDA-MB-231 in a murine host was determined. Taccalonolide AF was administered at a dose of 2.5 mg/kg on days 0 and 4 or 2.0 mg/kg on days 0, 3, and 7. These doses of taccalonolide AF were sufficient to observe antitumor activity compared to vehicle treated controls (FIG. 6). These doses and schedules of AF also had antitumor activity equivalent or greater than the positive control of 10 mg/kg paclitaxel administered on days 0, 2 and 4, and 7 (FIG. 6). This preliminary result demonstrates that taccalonolide AF has antitumor activity.

Referring to FIG. 6, Nude mice bearing bilateral MDA-MB-231 human breast tumors were treated with 2.0 mg/kg of AF on days 0, 3 and 7, 2.5 mg/kg AF on days 0 and 4, or 10 mg/kg PTX on days 0, 2, 4 and 7 as a positive control. Tumor volume was measured using calipers and mass calculated with the formula: Tumor mass (mg)=0.5×length (mm³)×width (mm³)₂. Median tumor mass with standard error of the mean (n=10) are graphically represented. *p<0.05, **p<0.01.

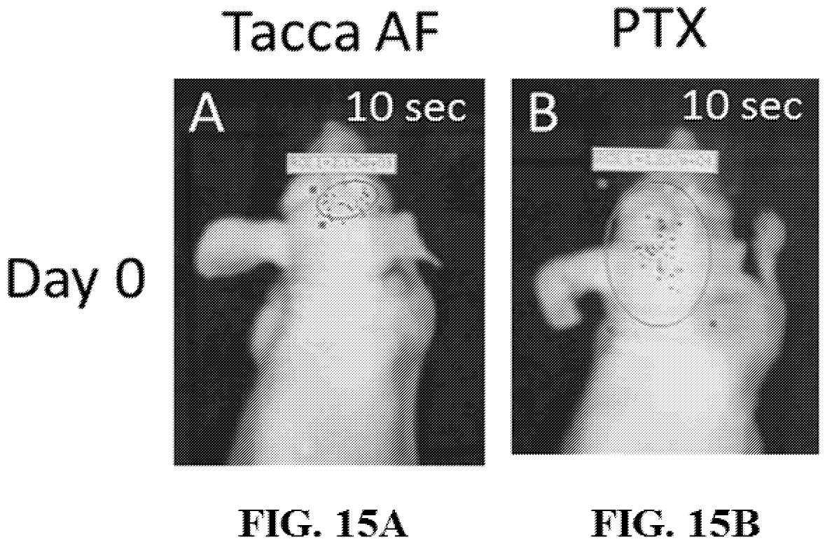

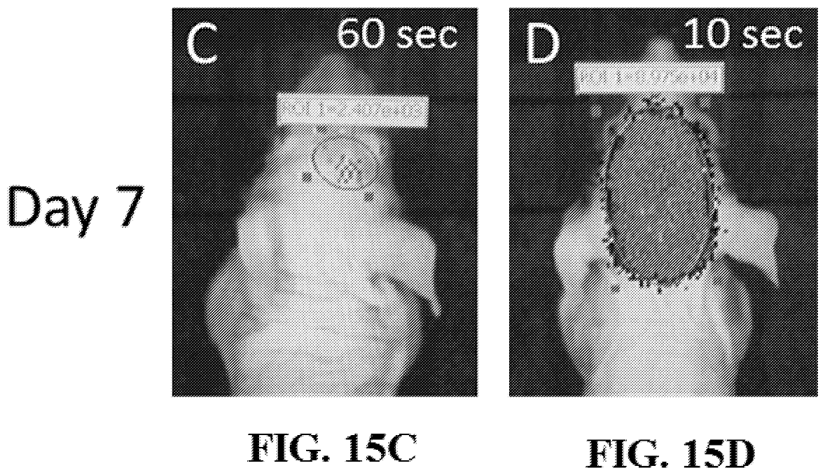

Referring to FIG. 15, A brain-seeking clone of the MDA-MB-231 triple negative breast cancer cell line stably transfected with luciferase was injected intracranially (1×10⁶ MDA-MB-231-BR-Luc2 cells in 5 μL PBS) into female athymic nude mice. Two weeks later (designated day 0), two mice had comparable tumor burdens as detected using the IVIS Spectrum in vivo imaging system 10 minutes after intraperitoneal injection of 100 μL of 57 mg/mL D-luciferin (FIGS. 15. A, B). One mouse (FIG. 15A) was injected intraperitoneally (ip) with 2.2 mg/kg taccalonolide AF and the other (FIG. 15B) with 20 mg/kg paclitaxel (ip) on days 0 and 4. On day 7, mice were again imaged as described above (FIGS. 15C, D). The brain tumor in the mouse treated with taccalonolide AF measured 2.2×10³ photon counts with an exposure time of 10 seconds on day 0 but was undetectable with the same 10 second exposure time on day 7. A longer exposure time of 60 seconds gave a photon count of 2.4×10³. The brain tumor in the mouse treated with paclitaxel measured 1.8×10⁴ photon counts with a 10 second exposure on day 0 and grew to 9.0×10⁴ photon counts with a 10 second exposure time on day 7.

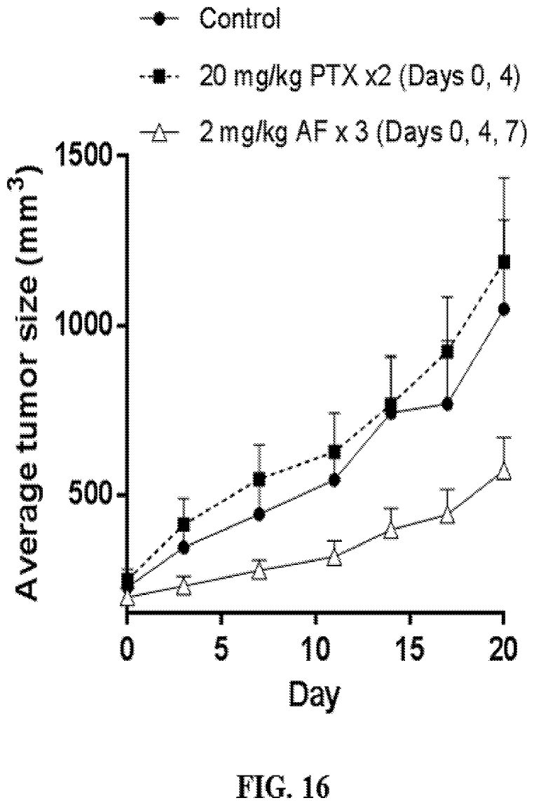

Referring to FIG. 16, Nude mice bearing bilateral NCI/ADR-RES human multi-drug resistant ovarian tumors were treated with 2 mg/kg taccalonolide AF on days 0, 4, and 7 and compared to treatment with 20 mg/kg paclitaxel on days 0 and 4 or to untreated control tumors. Tumor size was measured using calipers and volume calculated with the formula: Tumor volume (mm³)=width (mm)×length (mm)×height (mm) and graphed for days 0-20.

23. Efficacy of the Taccalonolides in Drug Resistant and Sensitive Cell Lines.

The ability of taccalonolides AF and AJ to inhibit the proliferation of both drug sensitive cancer cells, including ovarian cancer cells (SK-OV-3), cervical cancer cells (HeLa) and prostate cancer cells (PC-3) and drug resistant cells, including the P-glycoprotein expressing SK-OV-3 line (SK-OV-3/MDR-1-6/6) and the βIII-tubulin expressing HeLa cell line (WTβIII) was determined. IC₅₀ values were calculated for each cell line and the relative resistance of these cell lines to AF, AJ and paclitaxel (a drug that is susceptible to both modes of resistance) were determined by dividing the IC₅₀ of the drug resistant cell line by the IC₅₀ of the parental line. The relative resistance of taccalonolides AF and AJ in both cell line pairs was much lower than paclitaxel (Table 3), indicating that, like previously identified taccalonolides, the potent taccalonolides AF and AJ are able to circumvent clinically relevant drug resistance associated with either overexpression of P-glycoprotein or βIII-tubulin. Additionally, the ability of the taccalonolides AF and AJ to potently inhibit the proliferation of a variety of cancer cell lines, including ovarian, cervical and prostate lines, suggests they may have a broad efficacy against many types of cancer.

TABLE 3

| | | | Paclitaxel |
|---|---|---|---|
| | AF (nM) | AJ (nM) | (nM) |
| HeLa | 23.6 ± 2.1 | 6.6 ± 0.3 | 1.6 ± 0.1 |
| WTβIII | 30.6 ± 3.3 | 11.1 ± 0.6 | 17.8 ± 1.2 |
| (Rr) | (1.3) | (1.7) | (11.3) |
| SK-OV-3 | 79.4 ± 3.5 | 16.3 ± 0.8 | 3.8 ± 0.2 |
| SK-OV-3/ | 366 ± 30.6 | 126 ± 12.8 | 785 ± 88 |
| MDR-1-6/6 | | | |
| (Rr) | (4.6) | (7.8) | (207) |
| PC-3 | 128 ± 16 | 25.1 ± 4.0 | 3.7 ± 0.2 |

Referring to Table 3, the effect of the taccalonolides in drug resistant and sensitive cells is shown. IC₅₀ values for inhibition of cellular proliferation for taccalonolides AF and AJ were determined in drug sensitive and drug resistant cell lines. The HeLa cell pair evaluated the effect of βIII tubulin expression on cell sensitivity and the ability of compounds to overcome drug resistance mediated by βIII tubulin expression. The SK-OV-3 cell line pair was used to evaluate the effects of the expression of P-glycoprotein (Pgp) on cell sensitivity and the ability of compounds to overcome Pgp-mediated drug resistence. The effects of the taccalonolides on the drug senstive prostate cancer cell line PC-3 are also presented. IC₅₀ values were calculated from an average of 3-4 independent experiments, each performed in triplicate.

24. Taccalonolides AF and AJ are not Cytotoxic to Normal Cells

The taccalonolides AF and AJ were added to human mammary epithelial cells at concentrations 5 to 100-fold their IC₅₀ values in the HeLa cancer cell line. No cytotoxicity of these normal cells was observed at any of the concentrations tested, indicating that these new potent taccalonolides do not kill normal epithelial cells at concentrations two orders of magnitude greater than the concentration that causes significant antiproliferative effects in cancer cells.

25. Structure-Activity of the Taccalonolides

Preliminary structure-activity relationships of the taccalonolides has been described (Li et al., 2011, Peng et al., 2010, Risinger et al., 2008). Taccalonolide AF, which differs from taccalonolide A only by conversion of the C22-C23 double bond to an epoxide group, has an IC₅₀ value of 23 nM (Table 1), which is a 234-fold increase in potency as compared to taccalonolide A. The conversion of taccalonolide B to taccalonolide AJ by epoxidation at this same site resulted in a 743-fold increase in potency. The importance of the C22-C23 epoxide moeity to biological potency led to the epoxidation of 23 additional taccalonolides. Each of the taccalonolides with an epoxide group at C22-C23 was significantly more potent than the parent taccalonolide (Table 1). AI-epo, the epoxide product of taccalonolide AI, was the most potent taccalonolide generated with an IC₅₀ of 0.73 nM. These results indicate that an epoxide moiety at C22-C23 has a major impact on biological potency. Taccalonolide AC, which differs with taccalonolide A by an additional hydroperoxyl group at C20, showed no activity at concentrations as high as 50,000 nM. Taccalonolides AK and AO, both of which contain a six-member lactone ring and C23 carbonyl groups in place of the five-member lactone ring of other taccalonolides, showed no activity at concentrations as high as 30,000 nM. Taken together, these results highlight the importance of the C20-C22-C23 region of the taccalonolide molecule and suggest that this region plays a central role in its interaction with tubulin/microtubules.

The taccalonolides S, T, AG, AH, AI and AM, which all contain isobutyrate or isopentyrate groups at C1, are more potent than the taccalonolides E, R, AP, N and AL, which have an acyloxy group at C1. These results suggest that a bulky substituent at C1 is optimal for biological potency. Taccalonolides AQ, AR and AS, in which the C2-C3 epoxide ring has been opened and replaced with a chlorine group, showed little to no activity at concentrations as high as 30,000 nM, suggesting this epoxide is also critical for optimal potency. When an OH group was introduced at C5 to taccalonolides E, N and AI which lack a C11 acyloxy to form taccalonolides AP, AL and AM, respectively, a decrease in potency was observed.

Introducing an OH group at C5 in taccalonolides A and B, which have an acyloxy group at C11, to form taccalonolides Z and AB resulted in increased potency. These results indicate the importance of the 5-OH group for potency is related to the presence or absence of the 11-acyloxy moiety. Acetylation of the OH moeity at C11 also increased activity, which was evidenced by comparing taccalonolides AA and R with taccalonolides Z and AP (Table 1). The less potent taccalonolides E, N, R, AP and AL, which lack an 11-acyloxy group as compared to the more potent taccalonolides A, B, AA, Z and AB, further demonstrates that an 11-acyloxy group is optimal for taccalonolide potency.

Hydrolysis of the C15 acetate in taccalonolides A, E, AF, AH and AP, to the resulting taccalonolides B, N, AJ, AI and AL, resulted in more potent taccalonolides. Taccalonolide Z is an exception to this finding since hydrolysis of the C15 group, yielded taccalonolide AB, which was significantly less potent. Taccalonolide H2 is 7.4-fold more potent than taccalonolide A and differs only by the presence of an additional double bond in taccalonolide H2 at C7-C8. The location of this double bond is important, since a double bond at C5-C6 (as is found in taccalonolide AD) did not result in increased potency. When a hydroxyl group was added to the C7 of taccalonolide A to form the rare geminal diol in taccalonolide AE, the potency was also unchanged.

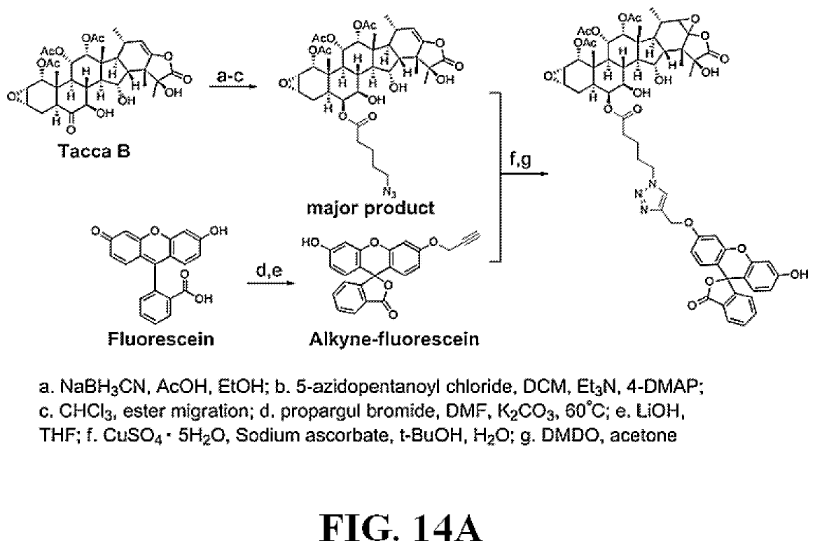

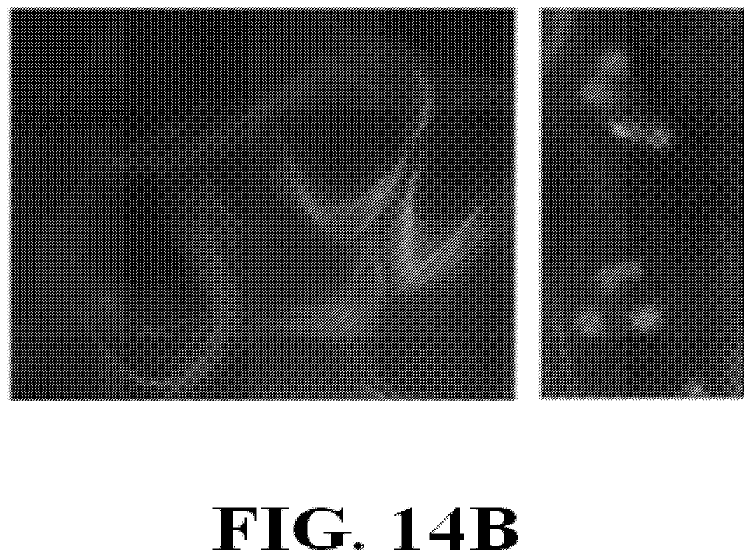

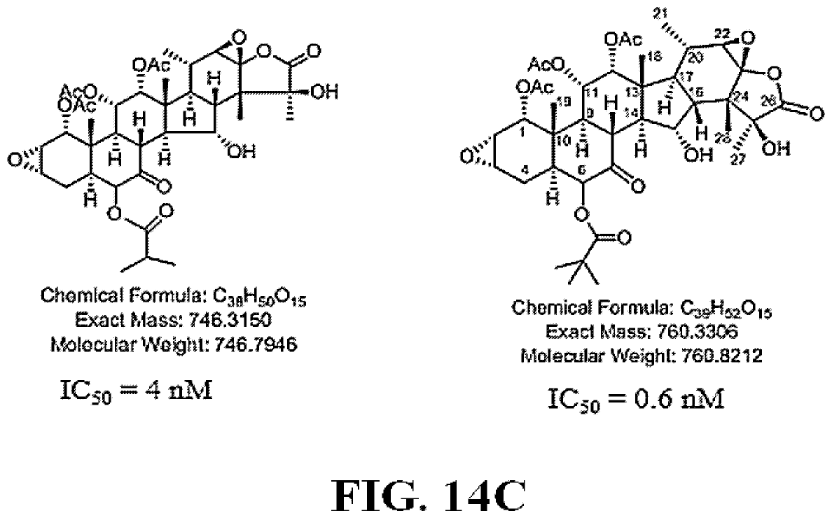

Referring to FIG. 14A-C, The C-6 moiety on the taccalonolide backbone was identified as a site that is amenable to the addition of linkers and probes. First generation C-6 biotin and fluorescein tagged taccalonolides that retain microtubule stabilizing activity were generated. Synthetic scheme to generate fluorescently-tagged taccalonolides through linkage at C-6, which can also be used to add other linkers (FIG. 14A). Localization of fluorescein-tagged AJ on microtubule bundles (left) and multipolar mitotic spindles (right) in HCC1937 cells treated with 5 μM of the C-6 fluorescein-labeled taccalonolide AJ conjugate for 4 h (FIG. 14B). Additional C-6-modified taccalonolides that retain microtubule stabilizing activity with low nM potency in the reference HeLa cell line (FIG. 14C).

The microtubule stabilizing activity of each taccalonolide correlates with its antiproliferative and cytotoxic potency, demonstrating that these properties of the taccalonolides are directly related to one another.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substituents and modifications apparent to those skilled in the art are deemed to be within the spirit, scope and concept of the invention as defined by the appended claims.

### K. REFERENCES

The following references, to the extent that they provide exemplary procedural or other details supplementary to those set forth herein, are specifically incorporated herein by reference:
Bennett et al., Chem. Biol., 17:725-734, 2010.
Boyd and Paull, Drug Develop. Res. 34:91-109, 1995.
Chen et al., Phytochem., 27:2999-3002, 1988.
Chen et al., Planta Medica, 63:40-43, 1997.
Chen et al., Tetrahedron Ltrs., 28:1673-1676, 1987.
Corbett et al., Cancer Treat. Rep., 62:1471-88, 1978.
Fojo and Menefee, Annual Oncol., 18 (5):v3-8, 2007.
Galsky et al., Nat. Rev. Drug Discov., 9(9):677-678, 2010.
Handbook of Pharmaceutical Salts: Properties, and Use (P. H. Stahl & C. G. Wermuth eds.), Verlag Helvetica Chimica Acta, 2002.
Huang and Liu, Helvetica Chimica Acta, 85:2553-2558, 2002.
Komlodi-Pasztor et al., Nat Rev Clin Oncol, 8:244-250, 2011.
Krishan, J. Cell Biol., 66:188-193, 1975.
Li et al., J. Am. Chem. Soc., 133:19064-19067, 2011.
Morris and Fomier, Clin. Cancer Res., 14(22):7167-7172.
Muhlbauer and Seip, Helvetica Chimica Acta, 86:2065-2072, 2003.
Nogales et al., Nature, 375:424-427, 1995.
Peng et al., J Med Chem Epub Aug. 11, 2011.
PCT Publn. WO/2001/040256
Polin et al., In: Transplantable Syngeneic Rodent Tumors: Solid Tumors of Mice, 2^{nd} Ed., Humana Press Inc., Totowa, N.J., 43-78, 2011.
Remington's Pharmaceutical Sciences, 15^{th} Ed., 1035-1038 and 1570-1580, 1990.
Remington's Pharmaceutical Sciences, 15^{th} Ed., 3:624-652, 1990.
Risinger et al., Cancer Res., 68:8881-8888, 2008.
Shen et al., Chinese J. Chem., 9:92-94, 1991.
Shen et al., Phytochem., 42:891-893, 1996.
Shen et al., J. Pharmacol. Exp. Ther. 337:423-432, 2011.
Skehan et al., J. Natl. Cancer Inst., 82:1107-1112, 1990.
Tinley et al., Cancer Res., 63:3211-3220, 2003.
Yang et al., Helvetica Chimica Acta, 91:1077-1082, 2008.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound of the formula: 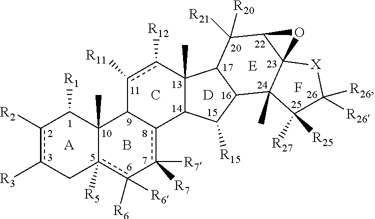
wherein:
R₁ is hydroxy, alkoxy_{(C≤12)} or acyloxy_{(C≤12)},
R₂ is hydroxy, halogen, or R₂ is taken together with R₃ to form an epoxide at C-2/C-3;
R₃ is hydroxy, halo, or R₂ is taken together with R₃ as defined above;
R₅ is hydrogen, hydroxy, amino, alkoxy_{(C≤9)}, alkylamino_{(C≤6)}, or dialkylamino_{(C≤12)};
R₆ is hydrogen, hydroxy, alkoxy_{(C≤30)}, acyloxy_{(C≤30)}, or oxo if R_{6′} is not present;
R_{6′} when present is hydrogen or hydroxy, alkoxy_{(C≤30)} or acyloxy_{(C≤30)};
R₇ is hydrogen, hydroxy, alkoxy_{(C≤30)}, acyloxy_{(C≤30)}, or oxo if R_{7′} is not present;
R_{7′} when present is hydrogen, hydroxy, alkoxy_{(C≤30)}, or acyloxy_{(C≤30)};
R₁₁ is hydrogen, hydroxy, alkyl_{(C≤6)}, alkoxy_{(C≤8)}, or acyloxy_{(C≤8)};
R₁₂ is hydrogen, hydroxy, alkyl_{(C≤6)}, alkoxy_{(C≤8)}, or acyloxy_{(C≤8)};
R₁₅ is hydrogen, hydroxy, alkyl_{(C≤30)}, alkoxy_{(C≤30)} or acyloxy_{(C≤30)};
R₂₀ is hydrogen, hydroxy, hydroperoxy, alkoxy_{(C≤8)} or acyloxy_{(C≤8)};
R₂₁ is hydrogen or alkyl_{(C≤6)};
R₂₅ is hydrogen, hydroxy, alkoxy_{(C≤8)} or acyloxy_{(C≤8)};
R₂₆ is hydrogen, hydroxy, alkoxy_{(C≤8)} or oxo if R_{26′} is not present;
R_{26′} when present is hydrogen, hydroxy or alkoxy_{(C≤8)};
R₂₇ is hydrogen or alkyl_{(C≤6)}; and
X is O, NR^{x} or CR^{x}₂, wherein each R^{x} is independently hydrogen or alkyl_{(C≤6)};
or a pharmaceutically acceptable salt thereof.

2. The compound of claim 1, wherein R₁ is acyloxy_{(C3-12)}.

3. The compound of claim 1, wherein C7/C8 are connected with a double bond.

4. The compound of claim 1, wherein R₅ is a hydroxy or alkyl_{(C≤6)}.

5. The compound of claim 1, further defined as:

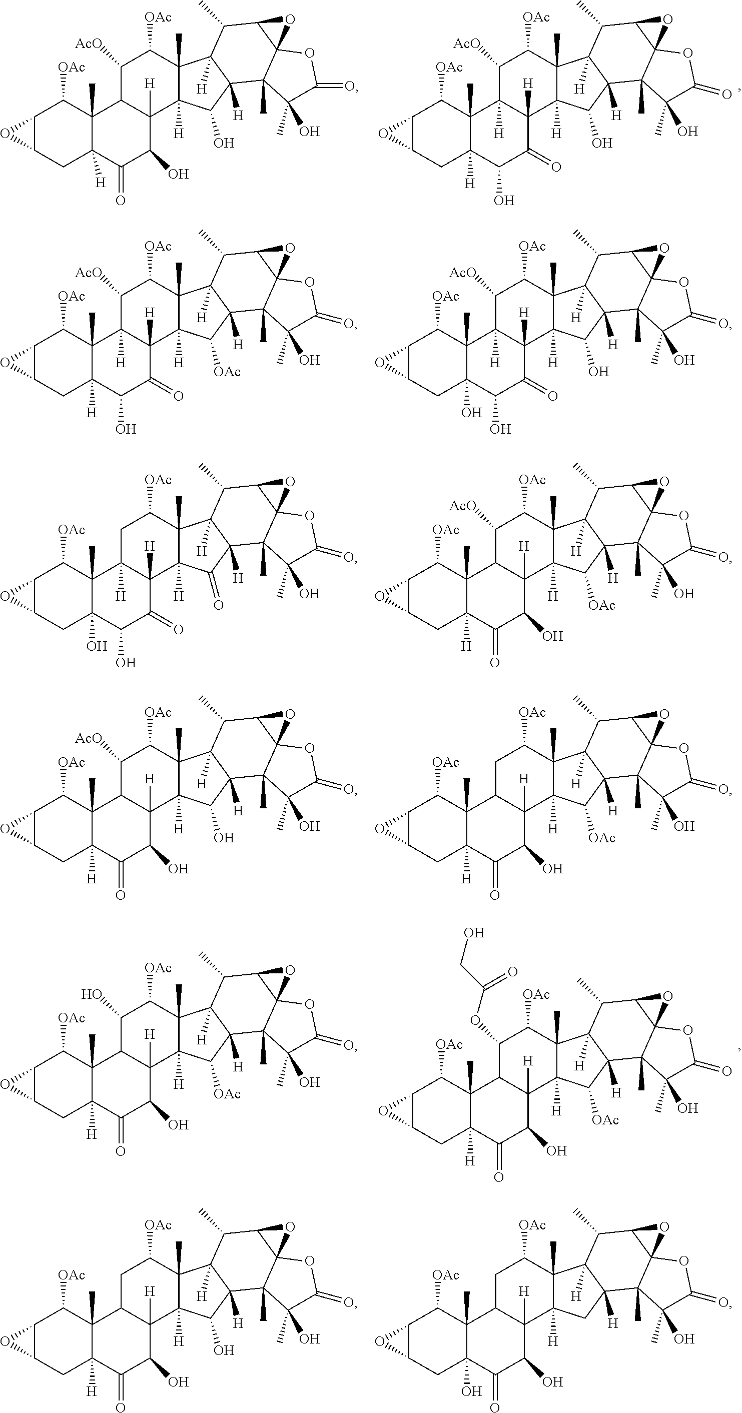

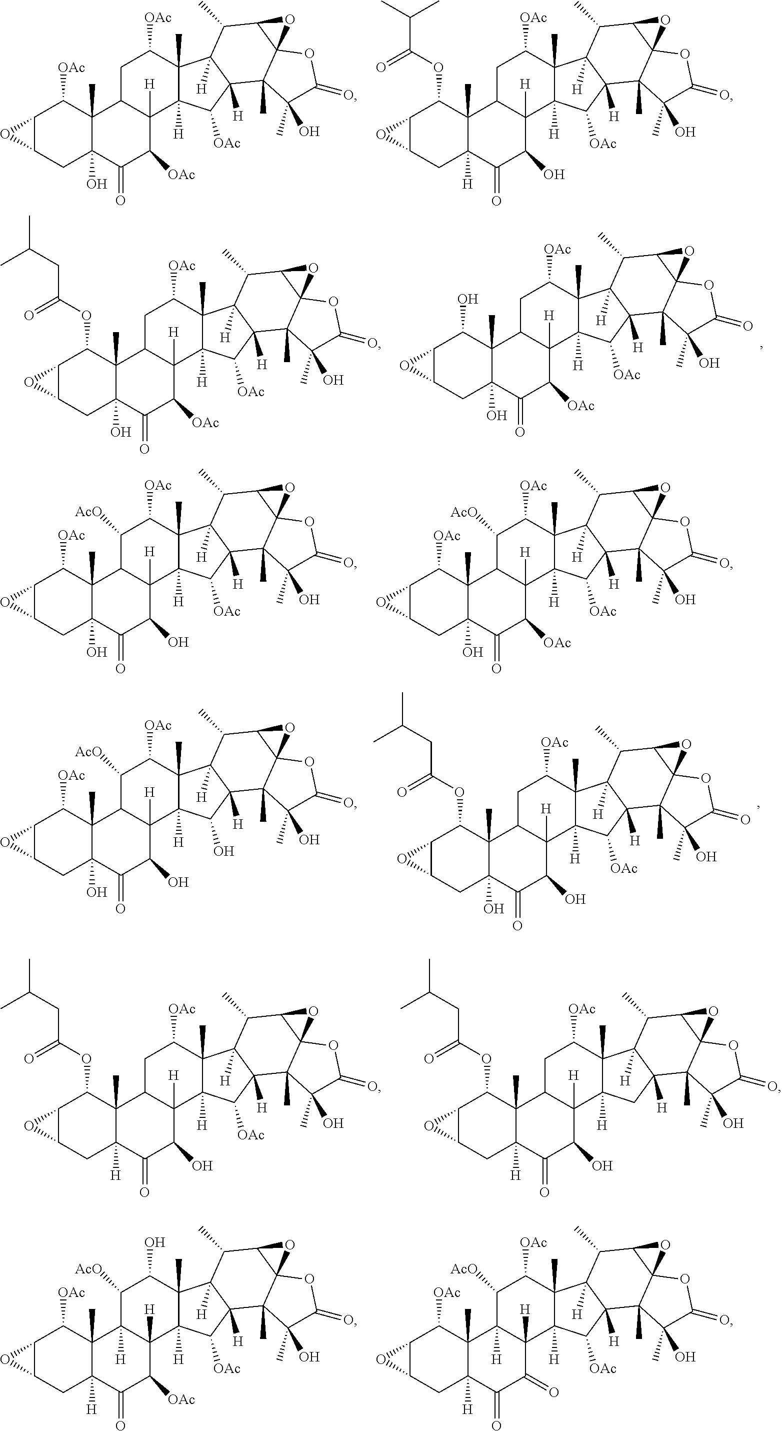

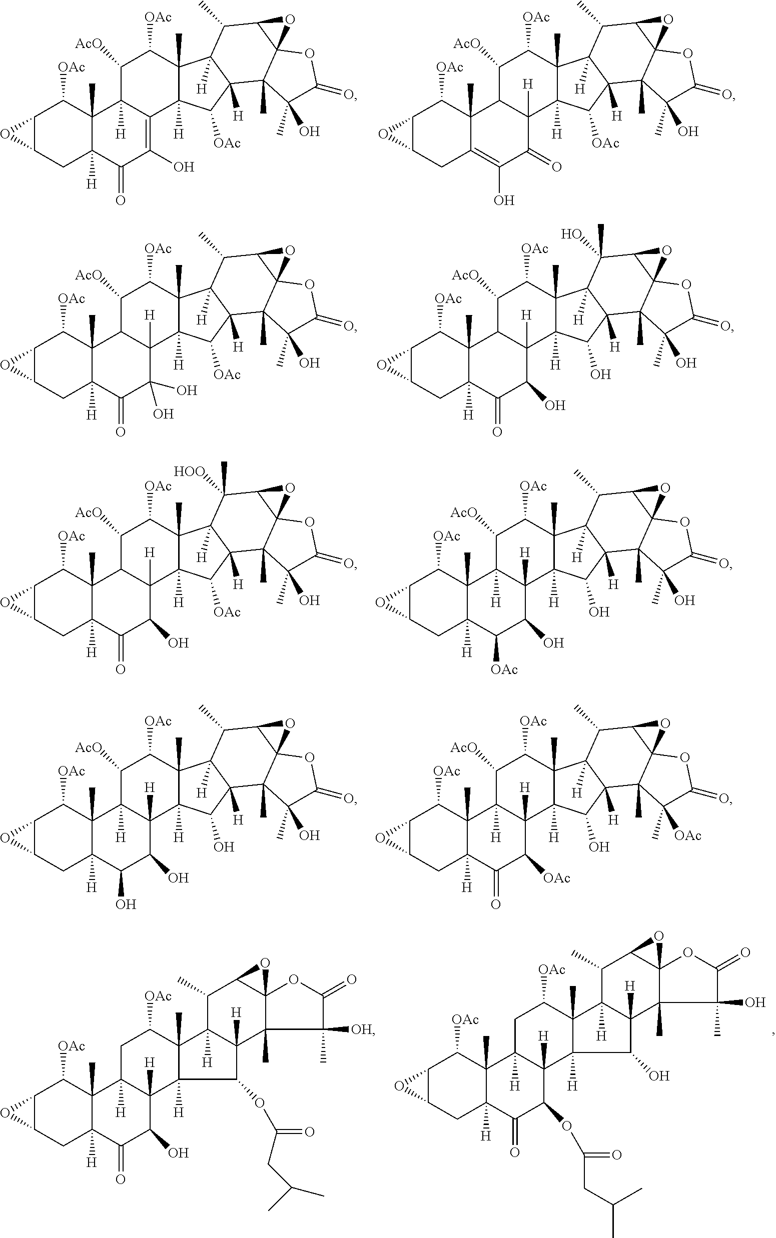

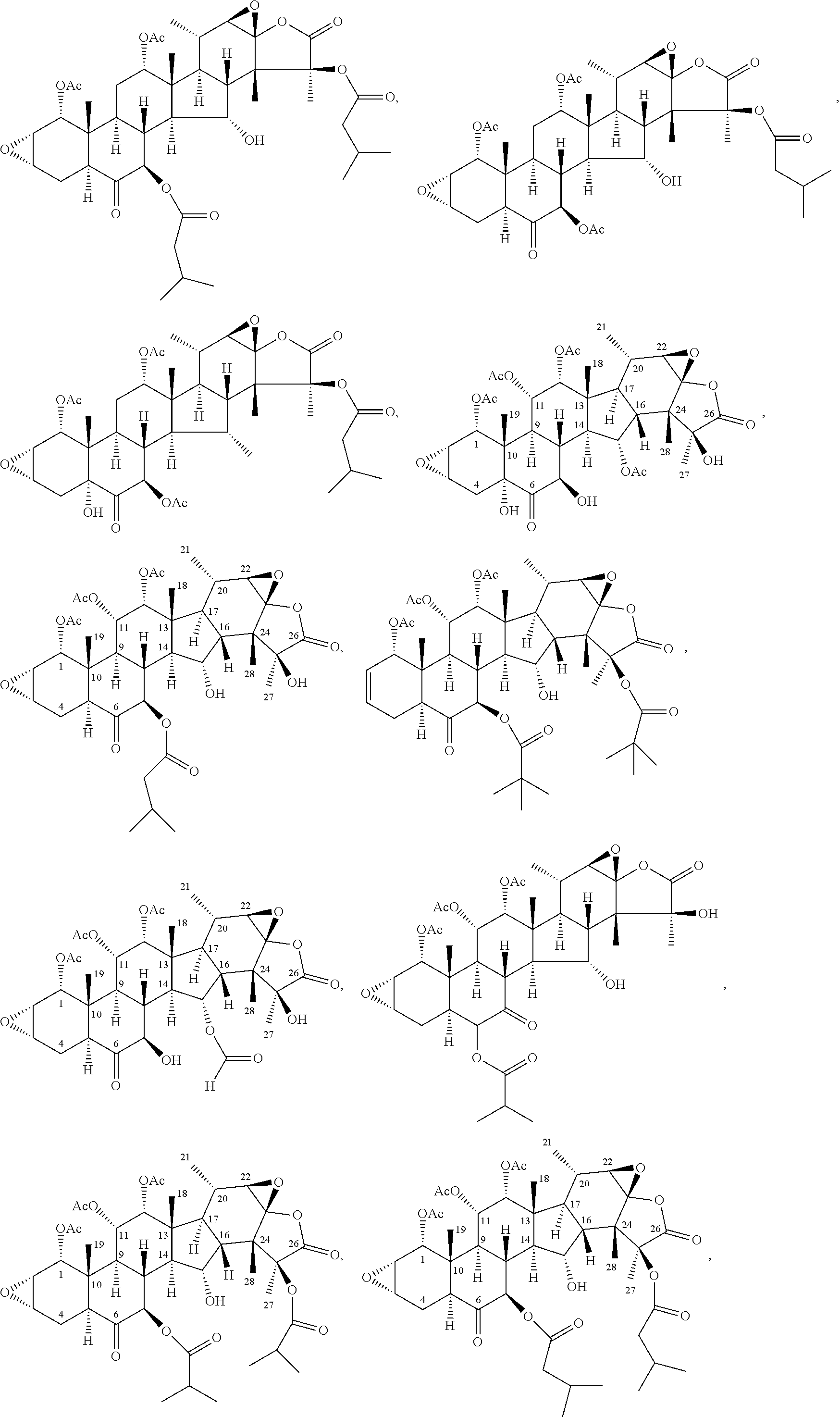

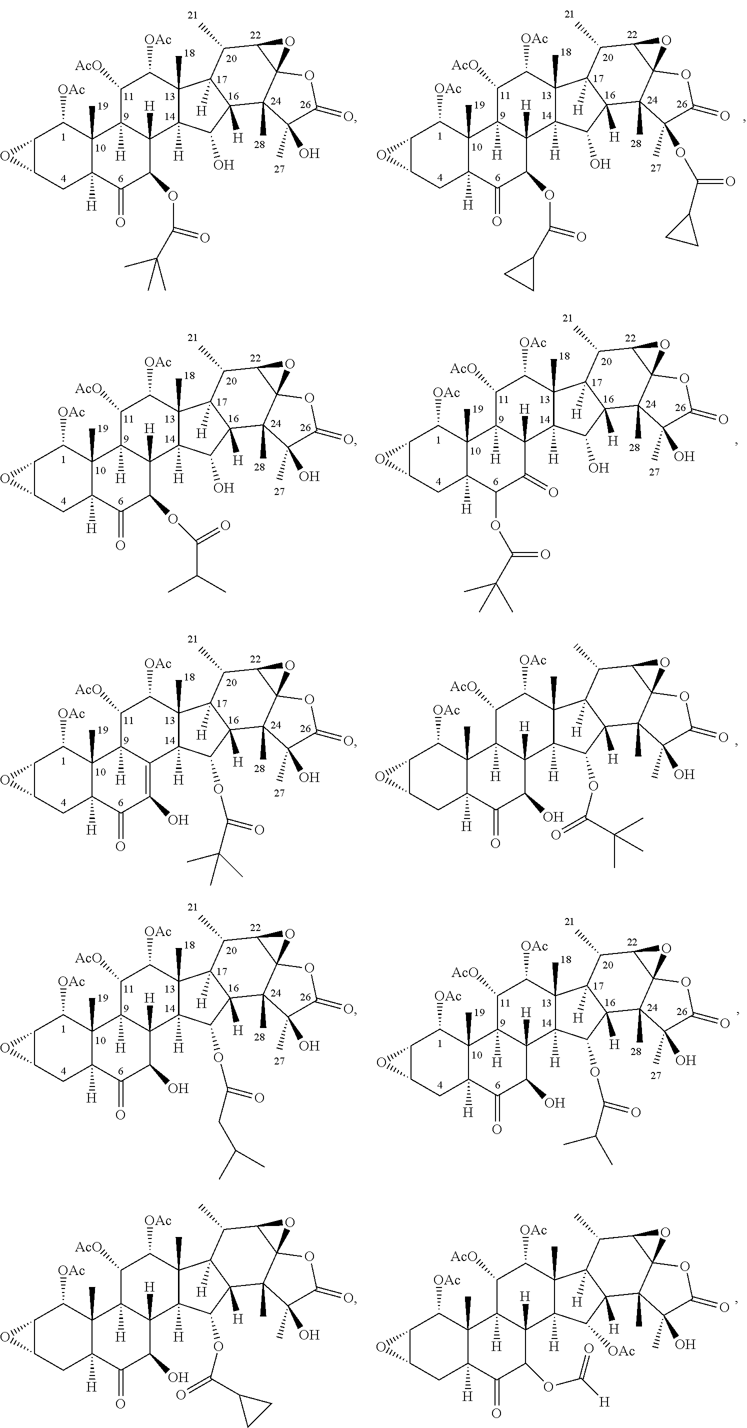

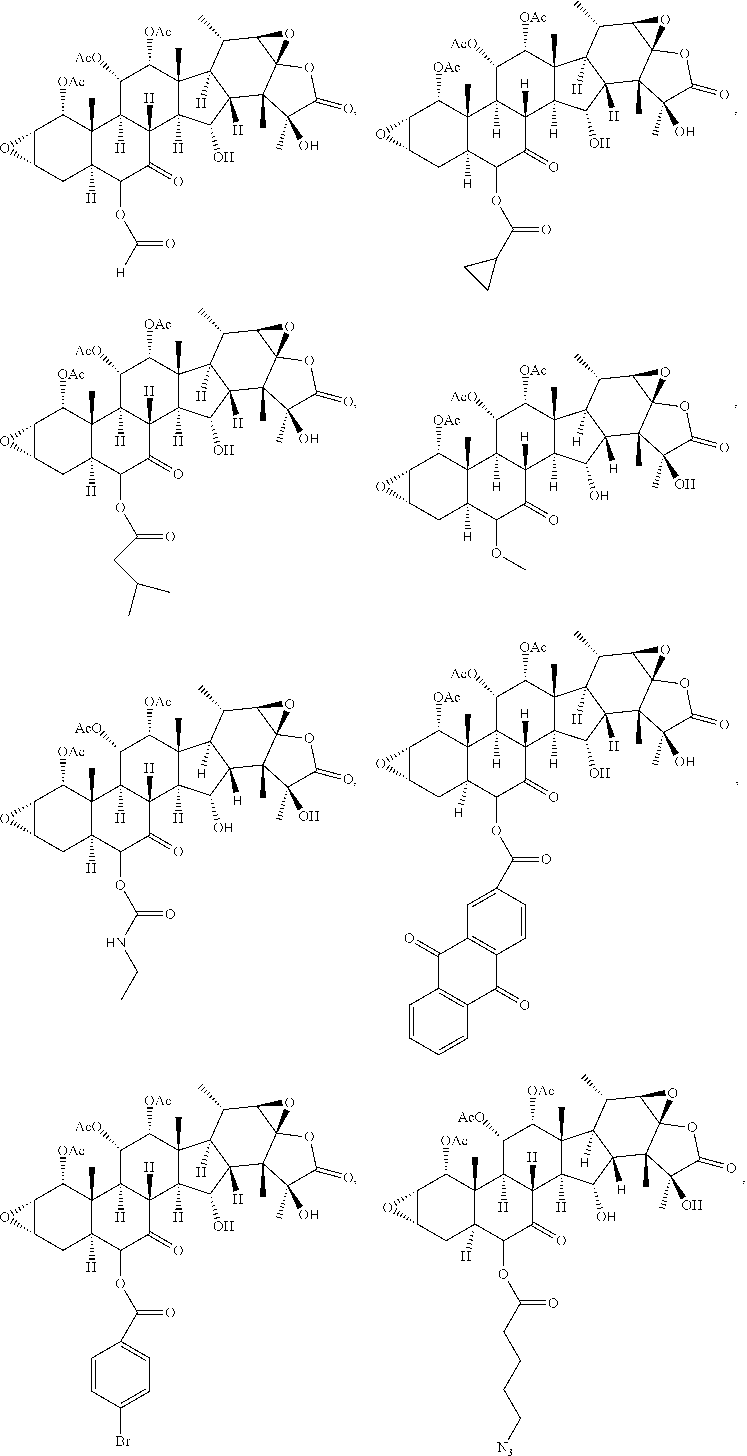

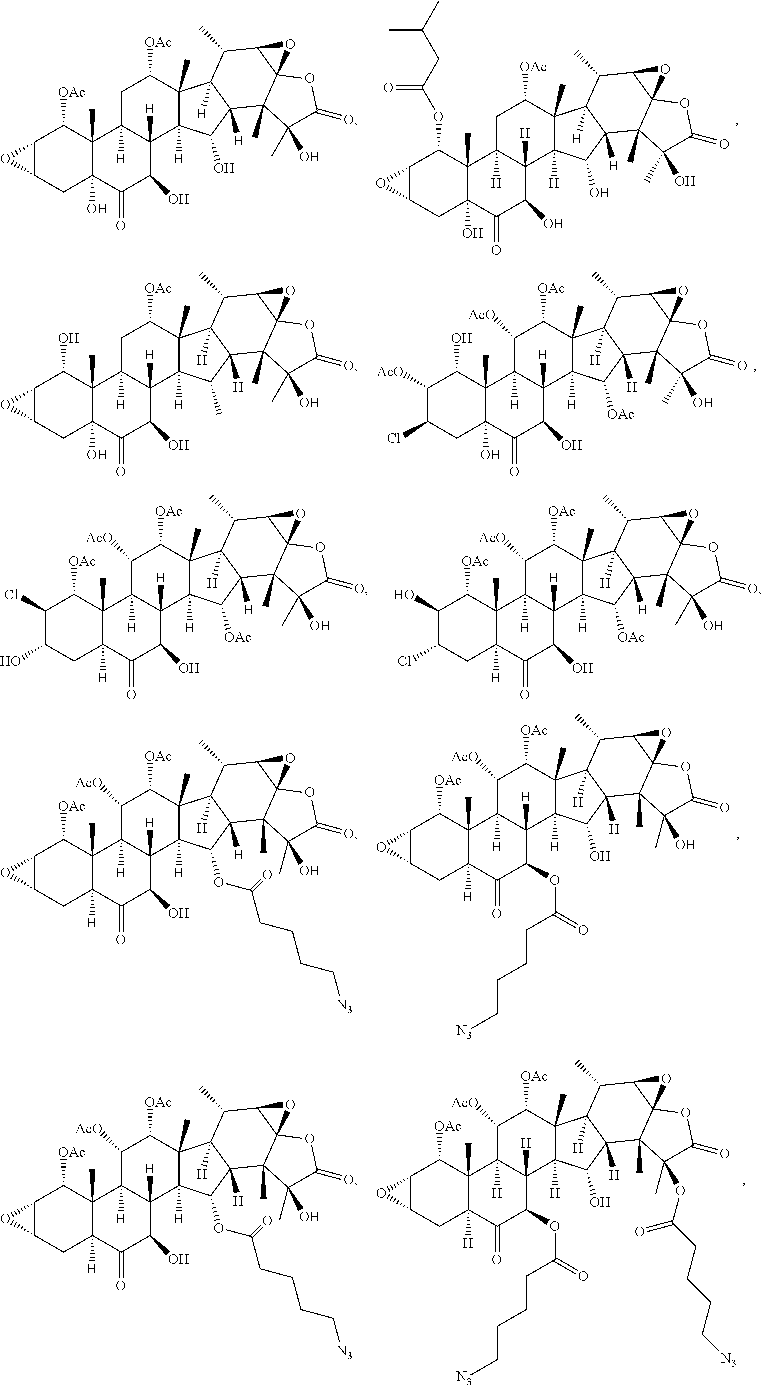

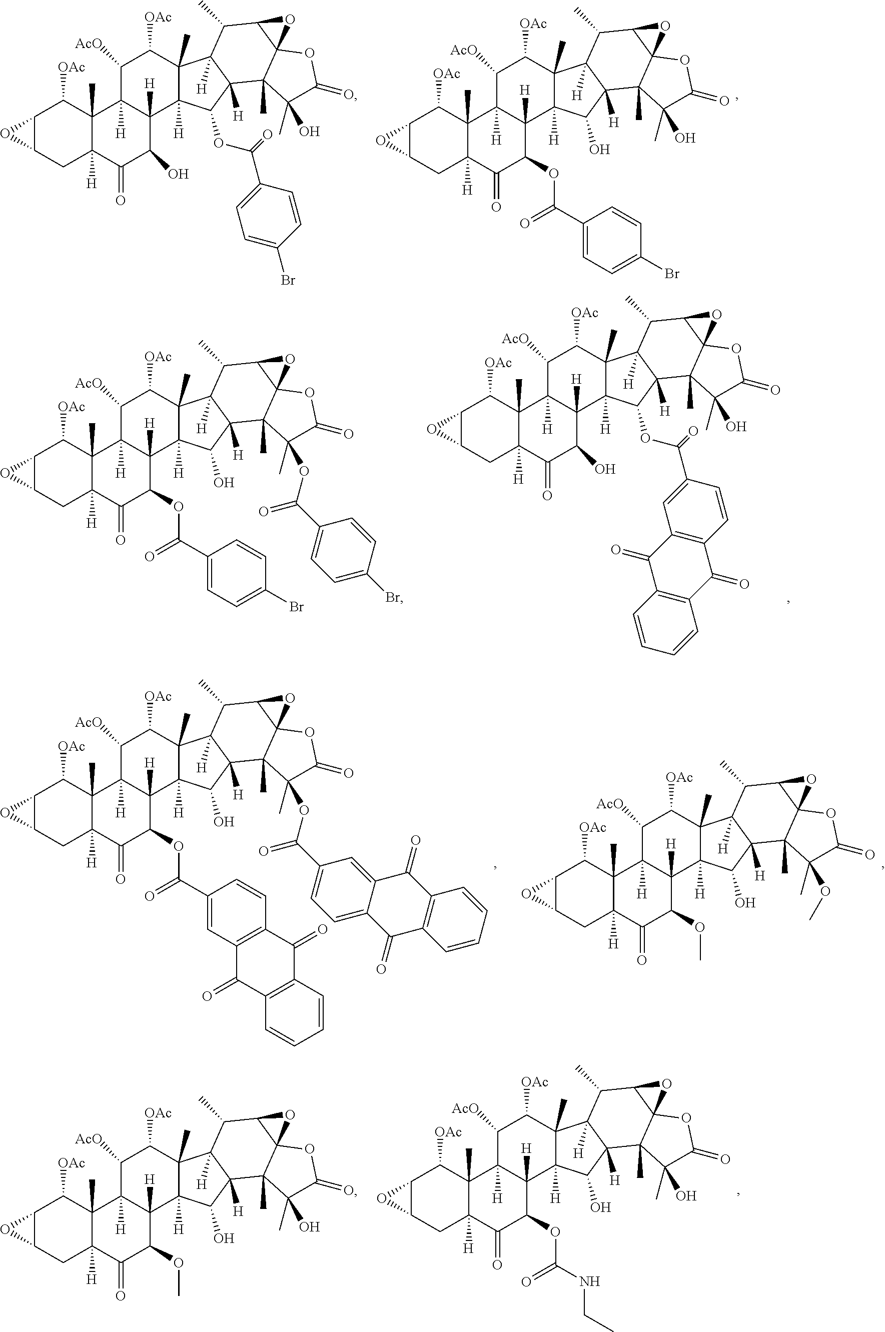

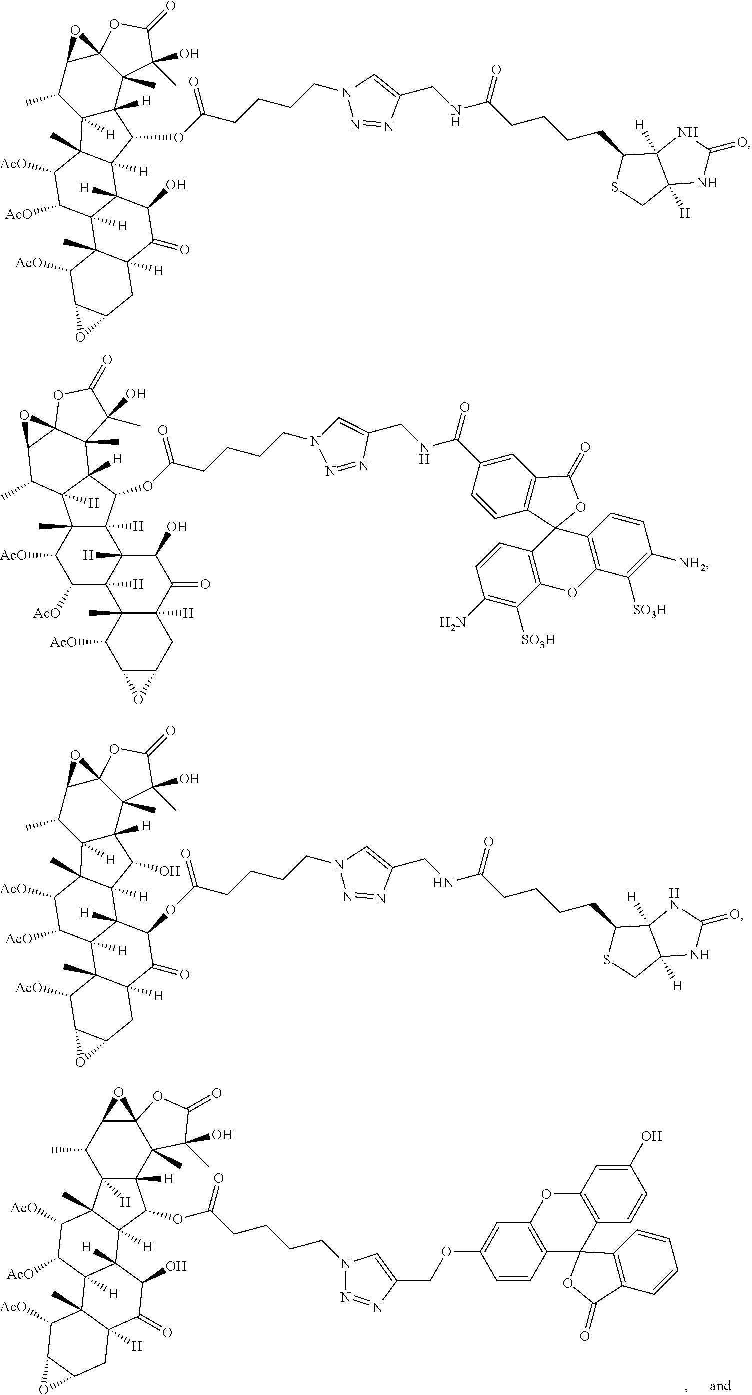

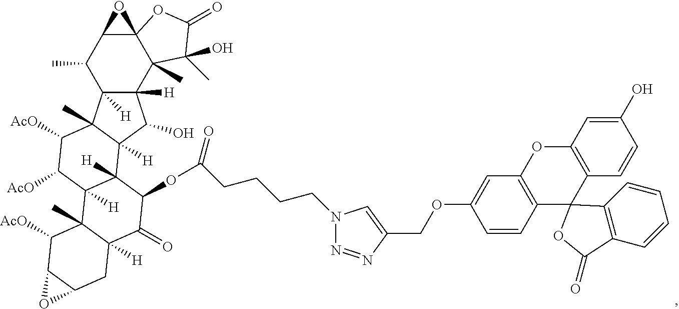

6. A compound having a structure represented by a formula: 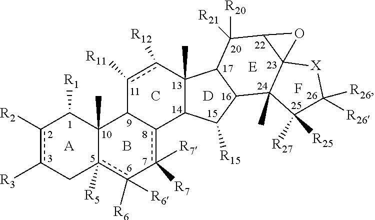
wherein each --- is an optional covalent bond;
wherein R₁ is selected from —OH, C1-C12 hydroxy, C1-C12 alkoxy, and —OC(O)(C1-C12 alkyl);
wherein each of R₂ and R₃ is independently selected from hydrogen, —OH, C1-C12 hydroxy, and halogen;
or wherein R₂ and R₃ together comprise —O—;
wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent;
wherein each of R₆ and R_{6′} is independently selected from hydrogen, —OH, C1-C30 hydroxy, C1-C30 alkoxy, C1-C30 acyloxy, —OC(O)Ar₁, —OC(O)Ar₂, —OC(O)(C1-C4 alkyl)Ar₂, and —OC(O)(C1-C8 azide);
wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
or wherein each of R₆ and R_{6′} together comprise ═O;
or wherein one of R₆ and R_{6′} is absent;
wherein each of R₇ and R_{7′} is independently selected from hydrogen, —OH, C1-C30 hydroxy, C1-C30 alkoxy, and C1-C30 acyloxy;
or wherein each of R₇ and R_{7′} together comprise ═O;
or wherein one of R₇ and R_{7′} is absent;
wherein each of R₁₁ and R₁₂ is independently selected from hydrogen, —OH, C1-C8 hydroxy, C1-C6 alkyl, C1-C8 alkoxy, and C1-C8 acyloxy;
wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar₂, —OC(O)(C1-C4 alkyl)Ar₂, and —OC(O)(C1-C8 azide);
wherein each of R₃₁ₐ and R_{31b}, when present, is independently selected from hydrogen and C1-C8 alkyl;
wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from: 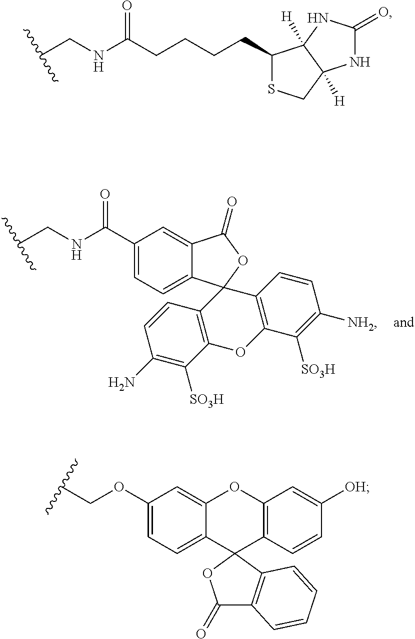
wherein R₂₀ is selected from hydrogen, —OH, —OOH, C1-C8 hydroxy, C1-C8 hydroperoxy, C1-C8 alkoxy, and C1-C8 acyloxy;
wherein R₂₁ is selected from hydrogen and C1-C6 alkyl;
wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar₁, and —OC(O)(C1-C8 azide);
wherein each of R₂₆ and R_{26′} is independently selected from hydrogen, —OH, C1-C8 hydroxy, and C1-C8 alkoxy;
or wherein each of R₂₆ and R_{26′} together comprise ═O;
wherein R₂₇ is selected from hydrogen and C1-C6 alkyl; and
wherein X is selected from O, NR^{x}, and CR^{x}₂;
wherein R^{x}, when present, is selected from hydrogen and C1-C6 alkyl,
or a pharmaceutically acceptable salt thereof.

7. The compound of claim 6, wherein the compound has a structure represented by a formula:

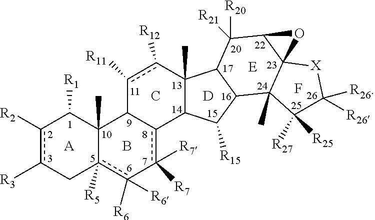

8. A compound having a structure represented by a formula: 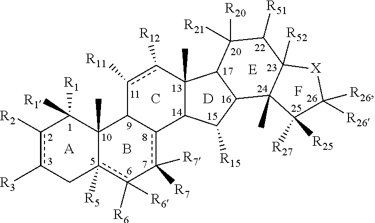
wherein each --- is an optional covalent bond;
wherein R₁ is selected from —OH, C1-C12 hydroxy, C1-C12 alkoxy, —OC(O)(C1-C12 alkyl), hydrogen, halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃, and wherein R₁ is hydrogen;
or wherein each of R₁ and R_{1′} together comprise ═O or ═NR₄₆;
wherein each of R₂ and R₃ is independently selected from hydrogen, —OH, C1-C12 hydroxy, and halogen, or wherein R₂ and R₃ together comprise an epoxide at C-2/C-3;
wherein R₅ is selected from hydrogen, —OH, —NH₂, C1-C6 alkyl, C1-C9 hydroxy, C1-C9 aminoalkyl, C1-C9 alkoxy, C1-C6 alkylamino, and (C1-C6)(C1-C6) dialkylamino, or wherein R₅ is absent;
wherein each of R₆ and R_{6′} is independently selected from hydrogen, —OH, C1-C30 hydroxy, C1-C30 alkoxy, C1-C30 acyloxy, —OC(O)Ar₁, —OC(O)Ar₂, —OC(O)(C1-C4 alkyl)Ar₂, —OC(O)(C1-C8 azide), halogen, —CN, —NC, —NCO, —OCN, —NO₂, —ONO₂, —ONO, —NO, —N₃, —NH₂, —NH₃, —N═NR₄₁, —NHOH, C1-C12 alkyl, C2-C12 alkenyl, C2-C12 alkynyl, C1-C12 thioalkyl, C1-C12 alkylthio, C1-C12 aminoalkyl, C1-C12 alkylamino, (C1-C12)(C1-C12) dialkylamino, —OP(O)(OR₄₂)₂, —OSO₂R₄₃, —C(O)(C1-C12 alkyl), —CO₂R₄₄, —C(O)NR₄₅ₐR_{45b}, —(C1-C12 alkyl)C(O)NR₄₅ₐR_{45b}, —OC(O)NR₄₅ₐR_{45b}, —(C1-C12 alkyl)OC(O)NR₄₅ₐR_{45b}, Cy₁, Ar₃, (C1-C12 alkyl)Ar₃, and —OAr₃;
or wherein each of R₆ and R_{6′} together comprise ═O or ═NR₄₆,
or wherein one of R₆ and R_{6′} is absent;
wherein R₇ is selected from hydrogen, —OH, C1-C30 hydroxy, C1-C30 alkoxy, C1-C30 acyloxy, and —OC(O)NR₃₁ₐR_{31b}, and wherein R_{7′} is selected from hydrogen, —OH, C1-C30 hydroxy, C1-C30 alkoxy, and C1-C30 acyloxy;
or wherein each of R₇ and R_{7′} together comprise ═O;
or wherein one of R₇ and R_{7′} is absent;
wherein each of R₁₁ and R₁₂ is independently selected from hydrogen, —OH, C1-C8 hydroxy, C1-C6 alkyl, C1-C8 alkoxy, and C1-C8 acyloxy;
wherein R₁₅ is selected from hydrogen, —OH, C1-C30 hydroxy, C1-C30 alkyl, C1-C30 alkoxy, C1-C30 acyloxy, —OC(O)NR₃₁ₐR_{31b}, —OC(O)Ar², —OC(O)(C1-C4 alkyl)Ar₂, —OC(O)(C1-C8 azide), and —OC(O)CH₃;
wherein R₂₀ is selected from hydrogen, —OH, —OOH, C1-C8 hydroxy, C1-C8 hydroperoxy, C1-C8 alkoxy, and C1-C8 acyloxy;
wherein R₂₁ is selected from hydrogen and C1-C6 alkyl;
wherein R₂₅ is selected from hydrogen, —OH, C1-C8 hydroxy, C1-C8 alkoxy, C1-C8 acyloxy, —OC(O)NR₃₁ₐR_{31b}, —OC(O)Ar₁, and —OC(O)(C1-C8 azide);
wherein each of R₂₆ and R_{26′} is independently selected from hydrogen, —OH, C1-C8 hydroxy, and C1-C8 alkoxy, or wherein each of R₂₆ and R_{26′} together comprise ═O;
wherein R₂₇ is selected from hydrogen and C1-C6 alkyl; and
wherein each occurrence of R₃₁ₐ and R_{31b}, when present, is independently selected from hydrogen and C1-C12 alkyl;
wherein each occurrence of R₄₁, R₄₂, R₄₄, R₄₅ₐ, and R_{45b}, when present, is independently selected from hydrogen and C1-C12 alkyl;
wherein each occurrence of R₄₃, when present, is independently selected from hydrogen, C1-C12 alkyl, and monocyclic aryl monosubstituted with a methyl group;
wherein each occurrence of R₄₆, when present, is independently selected from hydrogen and C1-C12 alkyl;
wherein each of R₅₁ and R₅₂ is independently halogen;
or wherein each of R₅₁ and R₅₂ together comprise —O— or —N(R⁵³)—;
wherein R₅₃, when present, is selected from hydrogen, C1-C4 alkyl, —SO₂R₅₄, and a structure having a formula: 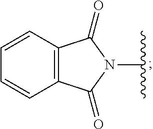
wherein R₅₄, when present, is selected from hydrogen, C1-C4 alkyl, —CH₂CH₂Si(CH₃)₃, and monocyclic aryl monosubstituted with a methyl group;
wherein each occurrence of Cy₁, when present, is independently heterocycloalkyl substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein Ar₁, when present, is selected from monocyclic 6-membered aryl and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein Ar₂, when present, is selected from monocyclic 6-membered aryl, triazolyl, and anthracene-9,10-dionyl, and is substituted with 0, 1, 2, or 3 groups independently selected from halogen, —OH, —NH₂, C1-C4 alkoxy, C1-C4 hydroxy, C1-C4 aminoalkyl, C1-C4 alkylamino, (C1-C4)(C1-C4) dialkylamino, and a structure represented by a formula selected from: 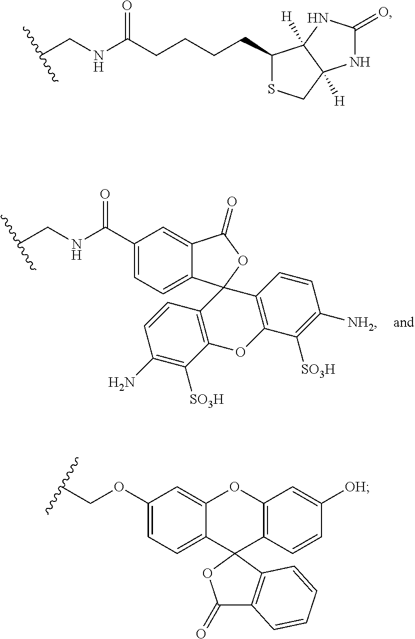
wherein each occurrence of Ar₃, when present, is independently selected from monocyclic aryl, morpholinyl, anilinyl, indolyl, pyrrolyl, imidazolyl, benzimidazolyl, pyrazolyl, guanidinyl, and piperazinyl and substituted with 0, 1, 2, or 3 groups independently selected from halogen, —OH, —NH₂, C1-C4 alkoxy, C1-C4 hydroxy, C1-C4 aminoalkyl, C1-C4 alkylamino, and (C1-C4)(C1-C4) dialkylamino;
wherein X is selected from O, NR^{x}, and CR^{x}₂;
wherein R^{x}, when present, is selected from hydrogen and C1-C6 alkyl,
or a pharmaceutically acceptable salt thereof.

9. The compound of claim 8, wherein the compound has a structure represented by a formula:

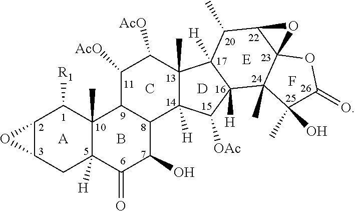

10. The compound of claim 8, wherein the compound has a structure represented by a formula:

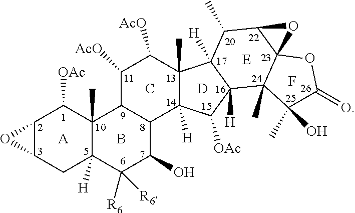

11. The compound of claim 8, wherein the compound has a structure represented by a formula: 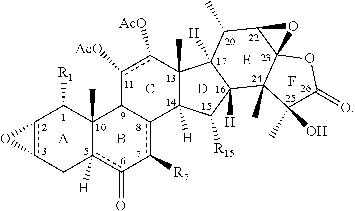
wherein R₇ is selected from —OH and —OC(O)NR₃₁ₐR_{31b}; and
wherein R₁₅ is selected from —OH, —OC(O)NR₃₁ₐR_{31b}, and —OC(O)CH₃.

12. The compound of claim 8, wherein the compound has a structure represented by a formula: 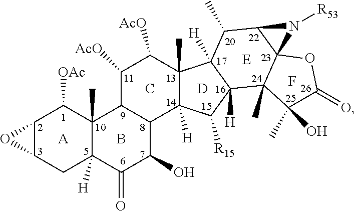
wherein R₁₅ is selected from —OH and —OC(O)CH₃; and
wherein R₅₃ is selected from hydrogen, methyl, —SO₂CH₂CH₂Si(CH₃)₃, and a structure selected from:

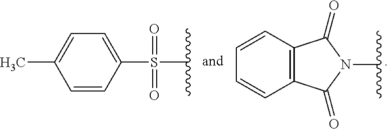 [SMILES: *S(=O)(=O)c1ccc(C)cc1 ; *N1C(=O)c2ccccc2C1=O]

13. The compound of claim 8, wherein the compound has a structure represented by a formula: 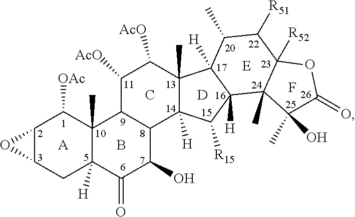
wherein R₁₅ is selected from —OH and —OC(O)CH₃; and
wherein each of R₅₁ and R₅₂ is halogen.

14. The compound of claim 8, wherein the compound is selected from:

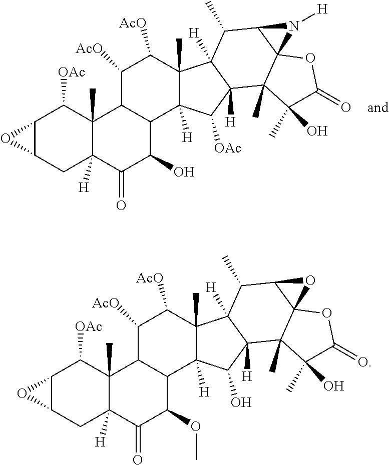

15. A composition comprising at least 90% by weight of a compound according to claim 1, claim 6, or claim 8.

16. A composition comprising a compound according to claim 1, claim 6, or claim 8 and a pharmaceutically acceptable carrier therefor.

17. A method of treating a hyperproliferative disorder in a patient, the method comprising administering to a patient in need thereof an effective amount of a compound according to claim 1, claim 6, or claim 8 or of an effective amount of a composition according to claim 16.

18. Use of a compound according to claim 1, claim 6, or claim 8 or of a composition according to claim 16 in the preparation of a medicament for the treatment of a hyperproliferative disorder in a patient.

19. A compound according to claim 1, claim 6, or claim 8 for the treatment of a hyperproliferative disorder in a patient.

20. A method of producing a mixture of epoxytaccalonolides, said method comprising subjecting a solution of a taccalonolide-containing crude extract of the roots and/or rhizomes of a Tacca species in an organic solvent to epoxidation.
